SEQUENCE LISTING (56 sequences)

SEQ ID NO: 1 - protein, 385 aa, Cannabis sativa
  misc_feature (1)..(385): B1Q2B6 OLIS_CANSA 3,5,7-trioxododecanoyl-CoA synthase
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 2 - protein, 392 aa, Oncidium hybrid cultivar
  misc_feature (1)..(392): AAZ32094.1
MPSLESVKNAKRAEGFASILAIGRANPEHFIEQSSYPDFFFRVTNSEHLVALKKKFQRICDKTAIRKRHFAWNEELLTTNSCFQTFMGNSLNVRQEFAIREIPKLGAQAANKAIQEWGQPKSRITHLIFCTTSGMDLPGADYQLTEILGLNPNVERVMLYQQGCFAGGTTLRLAKCLAESRKGARVLVVCAETTTVLFRAPSEEHQDDLVTQALFADGASALIVGADPDEAAHERASFIIVSTSQVLLPDSAGAIGGHVSEGGLLATLHRDVPQIVSKNVGKCLEEAFTPLGISDWNSIFWVPHPGGRAILDQIEERVGLKPEKLFISRHVLAEYGNMSSVCVHFALDEMRKRSAKEGKATTGEGLDWGVLFGFGPGLTVETVVLHSVPVTN

SEQ ID NO: 3 - protein, 390 aa, Cymbidium hybrid cultivar
  misc_feature (1)..(390): AIM58716.1
MPSLESVKKSNRADGFASILAIGRANPENFIEQSTYPDFFFRVTNSEHLVNLKKKFQRICDKTAIRKRHFVWNEELLNANPCLGTFMDNSLNVRQEFAIREIPKLGAEAATKAIQEWGQPKSRITHLIFCTTSGMDLPGADYQLTQILGLNPNIERVMLYQQGCFAGGTTLRLAKCLAESRKGARVLVVCAETTAVLFRAPSEEHQDDLVTQALFADGASALIVGADPDETAHERASFVIVSTSQVLLPDSAGAIGGHVSEGGLIATLHRDVPQIVSKNVGKCLEEAFTPLGISDWNSIFWVPHPGGRAILDQVEERVGLKPEKLIVSRHVLAEYGNMSSVCVHFALDEMRKRSKKEGKATTGEGLDWGVLFGFGPGLTVETVVLHSVPI

SEQ ID NO: 4 - protein, 390 aa, Dendrobium catenatum
  misc_feature (1)..(390): XP_020704098.1
MPSLESIKKAPRADGFASILAIGRANPENFIEQSAYPDLFFRITNSEHLVDLKNKFKRICDKTAIRKRHFVWTEEFITANPCFSTFMDKSLNIRQEVAIREIPKLGAEAATKAIQEWGQPKSRITHLIFCTTSGMDLPGADYQLTQILGLNPNVERVMLYQQGCFAGGTTIRLAKCLAESRKGARVLVVCAETTTVLFRGPSEEHQDDLVTQALFADGASALIVGADPDEAADEHASFVIVSTSQVLLPDSAGAIGGHVSEGGLLATLHRDVPQIVSKNVGKCLEEAFTPLGISDWNSIFWVPHPGGRAILDQVEESVGLKPEKLFISRHVLAEYGNMSSVCVHFALDEMRKRSAKEGKATTGEGLEWGVLFGFGPGVTVETVILRSVPI

SEQ ID NO: 5 - protein, 393 aa, Phalaenopsis equestris
  misc_feature (1)..(393): XP_020572025.1
MPSFPSVKKAPTAEGFASILAIGRANPENFIEQSAYPDFFFRVTNSEHLVDLKKKFQRICDKTAIRKRHFVWNEEFLTANPCFSTFMDKSLNVRQEVAIREIPKLGAKAATKAIEDWGQPKSRITHLIFCTTSGMDLPGADYQLTQILGLNPNVERVMLYQQGCFAGGTTLRLAKCLAESRKGARVLVVCAETTTVLFRAPSEEHQDDLVTQALFADGASAVIAVIVGADPDEAADERASFVIVSASQVLLPDSAGAIGGHVSEGGLLATLHRDVPQIVSKNVGKCLEEAFTPFGISDWNSIFWVPHPGGRAILDQVEERVGLKPEKLSVSRHVLAEYGNMSSVCVHFALDEMRKRSAKEGKATTGEGLEWGVLFGFGPGLTVETVVLQSVPI

SEQ ID NO: 6 - protein, 390 aa, Anoectochilus roxburghii
  misc_feature (1)..(390): QDX46968.1
MPSLESIRKAPRADGLASILAIGRANPDNFMEQSSFPDFFFRITGSDHLVDLKKKFQRICDRTAIRKRHFVWNEEFIKANPCFSTFMDNSLNVRQEVAIREIPKLGAEAATKAIKEWGQPKSRITHLIFCTTSGMDLPGADYQLTRILGLNPNVERVMLYQQGCFAGGTTLRLAKCLAESRKGARVLVVCAETTTVLFRAPSEEHQEDLVTQALFADGASAVIVGADPDEEAHEKASFVIFSTSQVLLPDSEGAIGGHVSEGGLLATLHRDVPQLVSKNVGKCLEEAFTPLGISDWNSIFWVPHPGGRAILDQIEERVGLKPEKLTTSRHVLAEYGNMSSVCVHFVLDEMRKKSSKEGKATTGEGLEWGVLFGFGPGLTVETVVLRSVPL

SEQ ID NO: 7 - protein, 390 aa, Phalaenopsis hybrid cultivar
  misc_feature (1)..(390): AAX54693.1
MPTIESIKKAPRAHGFASILAIGKANPENFIEQCHYPDFYFRVTSSEHLVDLKEKFQRMCDRTAIRKRHFVWNEDLLTANPCLRTYMDKSLNIRQEVAIREIPKLGAEAATKAIQEWGQPKSSITHLIFCTTSGMDLPGADFQLTQILGLNPNVERVMLYQQGCFAGGTTLRLAKCLAESREGARVLVVCAETTTVVFRAPSEEHQDDLVTQALFADGASAVIVGVDPNEAAHERASFIIVSASQVLLPDSAGAIGGHVSEGGLTATLHRDVPQIVSKNVGKCLEEAFTPFGISDWNSIFWVPHAGGRAILDQVEERVGLKPEKLSVSRHVLAEYGNMSSVCVHFALDEMRKKSAKEAKATTGEGLEWGVLFGFGPGLTVETVVLHSVPI

SEQ ID NO: 8 - protein, 390 aa, Dendrobium officinale
  misc_feature (1)..(390): QCO76957.1
MPSLESIRKAPRANGFASILAIGRANPENFIEQSTYPDFFFRITNSEHLVDLKKKFQRICDKTAIRKRHFVWNEEFITTNPCLHTFMDKSLDVRQEVAIREIPKLGAKAAAKAIQEWGQPKSRITHLIFCTTSGMDLPGADYQLTQILGLNPNVERVMLYQQGCFAGGTTLRLAKCLAESRKGARVLVVCAETTTVLFRGPSEEHQEDLVTQALFADGASALIVGADPDEAAHERASFVIVSTSQVLLPDSAGAIGGHVSEGGLLATLHRDVPKIVSKNVEKCLEEAFTPFGITDWNSIFWVPHPGGRAILDLVEERVGLKPEKLLVSRHVLAEYGNMSSVCVHFALDEMRKRSAIEGKATTGEGLEWGVVFGFGPGLTVETVVLRSVPL

SEQ ID NO: 9 - protein, 390 aa, Bletilla striata
  misc_feature (1)..(390): AHH25569.1
MPSLDSIKKAPRADGIASILAIGRANPDNIIEQSAYPDFYFRVTNSEHLVDLKKKFQRICEKTAIRKRHFVWNEEFLTSNPSFSTFMDKSLYVRQEVAIREIPKLGAKAATKAIEDWGQPKSRISHLIFCTTSGMDLPGADYQLTQILGLNPNVERLMLYEQGCFAGGTTLRLAKCLAESRKGARVLVVCAETTTVLFRAPSEEHQDDLVTQALFADGASALIVGADPDEAADERASFVIVSTSQVLLPDTAGAIGGHVSEGGLLATLHRDVPQIVTKNVGKCLEEAFTPFGISDWNSIFWVPHPGGRAILDQVEERVGLKPEKLSVSRHVLAEYGNMSSVCVHFALDEMRKRSANEGKPTTGEGLEWGVLFGFGPGLTVETVVLRSVPL

SEQ ID NO: 10 - protein, 391 aa, Oncidium hybrid cultivar
  misc_feature (1)..(391): AAZ32093.1
MPSLESIKNAPKTDGFASILAIGSANPENIIEQSTYPDFFFHLTNSEHLVDLKNKFQRICDKTAIRKRHFAWNEELLTANPCLRTFMDNSLNVRQEFVIREIPKLGAQAATKAIQEWGQSKSRITHLIFSTTSGMDLPGADYQLTQILGLNPNIERVMLYHQGCFAGGTTLRLTKSLAESRKGARVLVVCAETTSALTFRAPSEEHQDDLVVQALFGDGASALIVGADPDEAADERASFIIVSTSQVLLSDSAGAIGGHLSEGGLIVTLHRDVPQIVSKNVGNCLKEAFTPLGISDWNSIFWVPHPGGPAILDQIEERVGLNPEKLIISRHMLAEYGNMLGVSVHFALDEMRKRSAKEGKATTGEGLDWGVLLGIGPGITVETVVLHSVRI

SEQ ID NO: 11 - protein, 388 aa, Dendrobium catenatum
  misc_feature (1)..(388): XP_020704102.1
MPSLESIKKAPRADGFASILAIGRANPENFIEQSAYPDLFFRITKSEHLVDLKNKFKRICDKTAIRKRHFVWTEEFITANPCFSTFMEKSLNIRQEVAIREIPKLGAEAAAKAIQEWGQPKSRITHLIFCTRSGMGLPGPDYQLTQILGLNPNVERVMIYQQGCFAGGTTLRLAKCLAESHKGARVLVVCAETSTVLFRAPSMEHQEDLVTQALFADGASALIVGADPDETADEHASFVIVSTSQVLLPESAGAIGGHVSEGGFLPMIHRDVPQIVSKNIGKCLEEAFTPLGIMDWNSIFWVPHPGGRAILDQLDERVGLKPEKLFISRHVLKEYGNMSSASVHFALDEMRKWSAKEGTTGEGLEWGVLFGFGPGVTVDTVVLRSVPI

SEQ ID NO: 12 - protein, 394 aa, Bromheadia finlaysoniana
  misc_feature (1)..(394): CAA10514.1
MASQVSPPSINMAPKADGFASILAIGRANPKNFIEQSTFPDFFFRVTNTEHMVDLKKKFQRICDKTSIRKRHFIWNEELLTANPSLCTFMGNSLNLRHEVAVREIPKLGAEAATKAIQEWGQPKSFITHLVFCTTSGMDLPGADYQLTQILGLNLDIERVMLHQQGCFLGGTTLRLAKYLAESRKGARVLVVCAETTTEFFRAPSEEHQEDLVTQSLFGDGASALIVGADPHEGARERASFILVSSSQVLLANSAHAITGHVSEGGIKATLHRDVPQIISNNLGKCLEEAFTPLGISDWNSIFWVLHPGGRAILDQVEEKMGLEPEKLLISRHVLLEYGNMSSVCVHFALDEMRKRSSNEGKATTGEGLEWGVLFGFGPGLTIETVVLRSVSIS

SEQ ID NO: 13 - protein, 394 aa, Paphiopedilum micranthum
  misc_feature (1)..(394): AFU07718.1
MPGLENRKKVEAPIRAEGLATIMAIGRANPPNAMEQSTFPDFYFRVTNSEHLVGLKKKFQRICEKTAIRRRHFVWNEEILNANPCLRTHMEPSLNVRQKIAVAEIPKMGAEAASRAIEEWGQSKSRITHLIFCTTSGMDLPGADYQLTRILGLNPNVQRVMLYQQGCFAGGTVLRLAKCLAESQKGARVLVVCSETTAVLVRAPSEEYHDDLVTQALFADGASALIVGADPDEEAKERPIFTIVSTTQVILPDSDGAIGGHLGEGGLTATLHRDVPLIISKNVSKCLEEAFTPLGISDWNSIFWAPHPGGRAILDQVEERASLKPEKLWASRHVLTEYGNMSSVCVHFVLDEIRKRSAKEGKATTGEGFDWGVLFGFGPGLTVETVVLRSVPLN

SEQ ID NO: 14 - protein, 394 aa, Paphiopedilum purpuratum
  misc_feature (1)..(394): AFU07719.1
MPGLENRKKVEAPKRAEGLATILAIGRANPPNDMEQSTFPDFYFRVTNSEHLVSLKKKFERICEKTAIRRRHFVWNEEILNANPCLRTHMEPSLNVRQKIAVAEIPKLGAEAASRAIEEWGQPKSHITHLIFCTTSGMDLPGADYKLTRILGLNPNVQRVMLYQQGCFAGGTVLRLAKCFAESQKGARVLVVCSETTTVLVRAPSEDYQDDLVTQALFADGASALIVGADPDEEAKEQPIFTIVSATQVILPDSDGAIGGHLGEGGLTATLHRDVPLIISKNVSKCLEEAFGPLGISDWNSIFWAPHPGGRAILDQVEERVGLKPEKLWASRHVLAEYGNMSSVCVHFVLDEIRKRSTKEGKTTTGEGFDWGVLFGFGPGLTVETVILRSVPLN

SEQ ID NO: 15 - protein, 393 aa, Apostasia shenzhenica
  misc_feature (1)..(393): PKA53998.1
MPGLQIISKASSRAADGLAAILAIGRANPPNSMDQSSYPEFYFRVMDSDHLVDLKKKFQRICERTAIRKRHFVWNEELLRDNPCLRTFMDSSLNVRQKVAVAEIPKLGAAAAERAIEEWGQPRSGITHLIFCTTSGMDLPGADYQLTKILGLNADVQRVMLYQQGCFAGGTVLRLAKVLAESRKGARVLVVCAETTTVLIRAPSVEHQDDLVTQALFADGASALIVGADPVEEVNERPLFSIISASQVILPDSDGAIGGHLGEGGLTATLHRDVPLIISKNVSKCLEDAFSPLGISDWNSIFWAPHPGGRAILDQVEERVGLKPEKMWASRHVLAEYGNMSSVCVHFVLDEMRKRSAKEGKPTTGEGLEWGVLFGFGPGLTVETVVLRSHPIN

SEQ ID NO: 16 - protein, 394 aa, Paphiopedilum armeniacum
  misc_feature (1)..(394): AFU07709.1
MPGLENRKKVEAPKREEGLATIMAIGRANPPNAMEQSTFPDFYFRVTNSEHMVGLKKKFQRICEKTAIRRRHFVWNEEILNANPCMCTHMEPSLNVGQKIVVAEIPKLGAEAASRAIEVWGQPKSRITHLIFCTTSSMDLLGADYKITRILGLNLNVQRGMLYQQGCFAGGTVLRLAKCFTESQKGTRVLVVCSENAIILVRASSEDYQDDLVTQALFADGASALIVGEDPDEEAKERPIFTIISTTHVILPDSDGAIGGHLGEGGLMATLQRDVPFIISKNVNTCLEESFAPLVISDWNSVFWAPHSGGRAILDQVEERAGLKPEKLWASRHVLAKYGNMSSVCVHFVLDEIRKRSTKEGKTTTGEGFDWGVLFGFGLGLTVETVILRSVPLN

SEQ ID NO: 17 - protein, 393 aa, Apostasia shenzhenica
  misc_feature (1)..(393): PKA56805.1
MPGVEAVAQNISPARSDGLAAILAIGRANPPNIVEQSSFADLYFRLHNSEHLVDLKKKLQRICDRTAIRKRHFVWDEELLMANPCLRTVTEPSLNARQKVAITEIPKLGAAAATNAIAEWGRPKSDITHLIFCTTSGMDLPGADYQLIRLLGLNDNIQRIMLYQQGCFAGGTVLRLAKVLAESRRSARVLIVCAETTTVLVRSPSVENQDDLVTQALFADGASALIVGADPNAGEKPVFSVFSTSQVLLPDSDGAIGGHVGENGLTATLHRDVPAVISKNVGKCLEEAFTPLGISDWNSIFWAAHPGGRAILDQVEERVGLKPEKMWASRHVLAEYGNMSSVSVHFALDEIRRRSAKEGKATTGDGFEWGVLFGFGPGLTVETVVLRSAPISA

SEQ ID NO: 18 - protein, 382 aa, Dendrobium catenatum
  misc_feature (1)..(382): PKU71375.1
MAQRADGSASILAIGKATPENFLEQSTYPDYFFRVTNSQHLIDLKKKFQRICDKTSIRKRHFILNEELITKNPCLSKFMENSINTRLEIYAKEIQKLAVEAATKAIQEWGQPKSCITHLIFSTLSDPGLPCGDYHLLQTLGLSPNIERVVILQHGCFAGGTMLRLAKCLAESHKGARVLVVSAETTTMLFRGPSEEHQEDLITQALFADGASALIVGVNPNETIGERASFVITSASQVILPNSSHAITGHLSEGGIKATIHKDVPNLLSNNIGKILEEAFTPLGISNWNSIFWVVHPGGRAILDQLEERVGLKPEKLMISRHVLAEYGNLMGVCVHFVLDEMRKRSIDEGNTTTGEGLEWGVLLGFGLGVTIETIVLQSVSL

SEQ ID NO: 19 - protein, 393 aa, Paphiopedilum x areeanum
  misc_feature (1)..(393): AFU07710.1
MPGLENRKKVEVPKRAKDLATIMAIGRANPPNAVEQSTFPDFYFRVTNNEHLVGLMKKFQCICEKTVIRRRHFVWDEEILNANPCLRTHMEPSFNVRQKIAVAEIPKMGAEAASRAIKEWGQPKSRITYLIFYTMSGMDLPVTDYKLTRILNLNLNVYQVMLYQWDCIAGGTILCWAKCSAESQKGTRVLVVSSETTTVLVRAPSEDYHDDLVTQDLFGDGASALIVGADPDEEAKERPIFTIVSTSQVVLPDSDGVIGGHLSEGGLMATLHRDVALIISKNVSKCLEEAFVTLGISDWNSIFWAPHPCRRAILDQVEERVGLKPEKLWASRHVLAEFGNMTSVCVHFVLDEIHNRSTQGKTTTGEGFDWGVLFGFGPGLIVETVILRSVPLN

SEQ ID NO: 20 - protein, 387 aa, Phalaenopsis equestris
  misc_feature (1)..(387): XP_020591420.1
MPNMESIKKEDGLATIMAIGRALPPNSIDQNSFPDFYFRVHNSEHLMDLKNKFRRICERTAIRKRHFVWNEEVLKQNPCLRTFMEPSLNTRQEIVCSEIPKLGAEAARNAIREWGQPERSITHLIFCTTSGMNLPGADFEAAQILGLNHSVERVMLYQQGCFAGGTVLRLAKCLAESRRGARVLVICAESTTSLVRSPSREHQYDLIAQALFADGASALIIGTEPNAEAGERPIFSIFSTAQVTLPDSGDAIRGYLKEGGLIATLAKDVPLIISENIERCLQEAFGPLGISDWNSIFWAPHPGGRAILDGIEDKLGLKPEKLWAARHVLAEYGNMSSVCVHYILDEMRRRDVKNGKAPTGDGPEWGVLFGFGPGLTVETVVLRRLFL

SEQ ID NO: 21 - protein, 402 aa, Leersia perrieri
  misc_feature (1)..(402): ProtKB - A0A0D9WZ61
MAPVPANGAATATATTQEIRRAQRADGPATVLAIGTANPETYVSQDEYADYYFRITKSEHLPELKDKLRRICNKSGIDKRFMYVNEDVMEAHPEFADRNQSSLNARVEIASKAVPELAAAASAKAIAEWGRPATDITHLIFSTYSGVKAPSGDRLLASLLGLRPNVSRTTLSLHGCYGGGRALQLAKELAENNRGARVLVACAEMTLIAFYGPEVGCNDTIVGQALFGDGSGAVIVGADPVDAAGERPLFEMAFASQTTVPDSEGAITMQHTKGGMDYHIGSGIPEMLAGNIERCLADAFDSIGVAADWKDLFWAVHPGGRRILDLIEEALGLDNGAMASRQVLREFGNMSGTTVIFVLNELRRRFKANGAEGADWGALMAFGPGVTIETMLLRVAAGLKGN

SEQ ID NO: 22 - protein, 402 aa, Leersia perrieri
  misc_feature (1)..(402): ProtKB - A0A0D9W1R8
MGSQGEYYNKEGRGQAAILGIGSAVPPYELPQSSFPDYYFDVSNSNHRQDLKAKFAKICERTMIEKRCVYMSEEFLRSNPSVTAYSSPSIDVRQRLTDATVPELGAAAARVAIADWGRPASEISHLVMCSTVSGCMPGADYEVVKLLGLPLSTKRCMMYHIGCHGGGTVLRIAKDIAENNPGARVLVVCSEVISMALRGPSDSNMGNLVGQALFGDAGAAVVVGTDPVESCGERALFEMVAAMQDIIPDTEEMVAKLREDGLLYSLHRDVPVHIEANIEAIVNKSGVGAKAADWNEDVFWLVHPGGRDILDRVARRLGLRHDKVEVSREVMRKHGNTLSSCVVIAMEEMRRRSAERGMGTAGEGLEWGLLFGFGPGLTVETILLRAPLTHPPPCAHAAVRPQ

SEQ ID NO: 23 - protein, 392 aa, Marchantia polymorpha
  misc_feature (1)..(392): Genbank Q5I6Y1
MSRSRLIAQAVGPATVLAMGKAVPANVFEQATYPDFFFNITNSNDKPALKAKFQRICDKSGIKKRHFYLDQKILESNPAMCTYMETSLNCRQEIAVAQVPKLAKEASMNAIKEWGRPKSEITHIVMATTSGVNMPGAELATAKLLGLRPNVRRVMMYQQGCFAGATVLRVAKDLAENNAGARVLAICSEVTAVTFRAPSETHIDGLVGSALFGDGAAAVIVGSDPRPGIERPIYEMHWAGEMVLPESDGAIDGHLTEAGLVFHLLKDVPGLITKNIGGFLKDTKNLVGASSWNELFWAVHPGGPAILDQVEAKLELEKGKFQASRDILSDYGNMSSASVLFVLDRVRERSLESNKSTFGEGSEWGFLIGFGPGLTVETLLLRALPLQQAERV

SEQ ID NO: 24 - protein, 411 aa, Marchantia polymorpha subsp. ruderalis
  misc_feature (1)..(411): Genbank A0A176WB84
MAPQAVDATCAAETASAVPMSRPRLIAQAVGPATVLAMGKAVPANIFEQATYPDFFFNITNSNDKPALKAKFQRICDKSGIKKRHFYLDQKILESNPAMCSYMETSLNCRQEIAIAQVPKLAKEASLNAIKEWGRPKSEITHIVMATTSGVNMPGAELATAKLLGLRPNVRRVMMYQQGCFAGATVLRVAKDLAENNAGARVLAICSEVTAVTFRAPCETHIDGLVGSALFGDGAAAVIVGSDPRPGIERPMYEMHWAGEMVLPDSDGAIDGHLTEAGLVFHLLKDVPGLITKNIGGFLKDTKNLVGASSWNELFWAVHPGGPAILDQVEAKLELEKGKFQASRDILSDYGNMSSASVLFVLDRVRERSLESNKSTFGEGNEWGFLIGFGPGLTVETLLLRALPMQQAESV

SEQ ID NO: 25 - protein, 411 aa, Marchantia polymorpha; identical to SEQ ID NO: 24
  misc_feature (1)..(411): Genbank A0A2R6XE22

SEQ ID NO: 26 - protein, 392 aa, Marchantia paleacea
  misc_feature (1)..(392): Genbank A0A2P0VPI9
MSRSRLIAQAVGPATVLAMGKAVPANVFEQATYPDFFFNITNSNDKPALKAKFQRICDKSGIKKRHFYLDQKILESNPAMCTYMETSLNCRQEIAVAQVPKLAKEASTNAIKEWGRPKSEITHIVMATTSGVNMPGAELATAKLLGLRPNVRRVMMYQQGCFAGATVLRVAKDLAENNAGARVLAICSEVTAVTFRAPSETHIDGLVGSALFGDGAAAVIVGSDPRPGIERPIYEMHWAGEMVLPESDGAIDGHLTEAGLVFHLLKDVPGLITKNIGGFLKDTKNLVGASSWNELFWAVHPGGPAILDQVEAKLELEKGKFQASRDILSDYGNMSSASVLFVLDRVRERSLESNKSTFGEGSEWGFLIGFGPGLTVETLLLRALPLQQAERV

SEQ ID NO: 27 - protein, 411 aa, Plagiochasma appendiculatum
  misc_feature (1)..(411): Genbank AHY39238
MAPQAVDAACAAEATPVIPTSRPRLIAQAVGPATVLAMGKAVPHNVFEQATYPDFFFNITKCNDKPTLKAKFQRICDKSGIKKRHFYLDQKILESSPSMCTYMETSLNCRQEIAIAQVPKLAKEAAQVAIKEWGRPKSEITHIVMATTSGVNMPGAELATAKLLGLRPNVRRVMMYQQGCFAGATVLRVAKDLAENNAGARVLAICSEVTAVTFRAPCETHIDGLVGSALFGDGAAAVIVGADPRPEIERPMFEMHWAGEMVLPESDGAIDGHLTEAGLVFHLLKDVPGLITKNIGGFLKDTKNLVGASTWNDLFWAVHPGGPAILDQVEAKLELEKNKFQASRDILADYGNMSSASVLFVLDRVRERSLESNKYTFGEGSEWGFLIGFGPGLTVETLLLRALPTEQAASA

SEQ ID NO: 28 - protein, 411 aa, Plagiochasma appendiculatum
  misc_feature (1)..(411): Genbank AIV42295
MAPQAVDAACAAEATPVIPTSRPRLIAQAVGPATVLAMGKAVPHNVFEQATYPDFFFNITKCNDKPTLKAKFQRICDKSGIKKRHFYLDQKILESNPSMCTYMETSLNCRQEIAIAHVPKLAKEAAQVAIKEWGRPKSEITHIVMATTSGVNMPGAELATAKLLGLRPNVRRVMMYQQGCFAGATVLRVAKDLAENNAGARVLAICSEVTAVTFRAPCETHIDGLVGSALFGDGAAAVIVGADPRPEIERPMFEMHWAGEMVLPESDGAIDGHLTEAGLVFHLLKDVPGLITKNIGGFLKDTKNLVGASTWNDLFWAVHPGGPAILDQVEAKLELEKNKFQASRDILADYGNMSSASVLFVLDRVRERSLESNKYTFGEGSEWGFLIGFGPGLTVETLLLRALPTEQAASA

SEQ ID NO: 29 - protein, 399 aa, Hydrangea macrophylla
  misc_feature (1)..(399): Genbank AAN76182
MATKSVAVEEMCKAQKAGGPATILAIGTAVPSNCYYQSEYPDFYFRVTKSDHLTDLKSKFKRMCDRSSIKKRYMHLTEEILKENPNMCSFAAPSIDGRQDIVVKEIPKLAKEAASKAIKEWGQPESNITHLVFCTTSGVDMPGCDYQLTRLLGLRPSIKRLMMYQQGCHAGGTGLRLAKDLAENNKGARVLVVCSEMTVINFRGPSEAHMDSLVGQSLFGDGASAVIVGSDPDLSTEHPLYQIMSASQIIVADSEGVIDGHLRQEGLTFHLRKDVPSLVSDNIENTLVEAFTPILMDSIDSIIDWNSIFWIAHPGGPAILNQVQAKVGLKEEKLRVSRHILSEYGNMSSACVFFIMDEMRKRSVEEGKGTTGEGLEWGVLFGFGPGFTVETIVLHSVPI

SEQ ID NO: 30 - protein, 399 aa, Hydrangea macrophylla
  misc_feature (1)..(399): Genbank AAN76183
MATKSVAVEEMCKAQKAGGPATILAIGTAVPSNCYYQSEYPDFYFRVTKSDHLTDLKSKFKRMCERSSITKRYMHLTEEILEENPNMCTFAAPSIDGRQDIVVKEIPKLAKEAASKAIKEWGQPKSNITHLVFCTTSGVDMPGCDYQLTRLLGLRPSIKRLMMYQQGCHAGGTGLRLAKDLAENNKGARVLVVCSEMTVINFRGPSEAHMDSLVGQSLFGDGASAVIVGSDPDLSTEHPLYQIMSASQIIVADSEGAIDGHLRQEGLTFHLRKDVPSLVSDNIENTLVEAFTPILMDSIDSIIDWNSIFWIAHPGGPAILNQVQAKVGLKEEKLRVSRHILSEYGNMSSACVFFIMDEMRKRSVEEGKGTTGEGLEWGVLFGFGPGFTVETIVLHSVPI

SEQ ID NO: 31 - protein, 393 aa, Pinus sylvestris
  misc_feature (1)..(393): Genbank Q02323
MGGVDFEGFRKLQRADGFASILAIGTANPPNAVDQSTYPDFYFRITGNEHNTELKDKFKRICERSAIKQRYMYLTEEILKKNPDVCAFVEVPSLDARQAMLAMEVPRLAKEAAEKAIQEWGQSKSGITHLIFCSTTTPDLPGADFEVAKLLGLHPSVKRVGVFQHGCFAGGTVLRMAKDLAENNRGARVLVICSETTAVTFRGPSETHLDSLVGQALFGDGASALIVGADPIPQVEKACFEIVWTAQTVVPNSEGAIGGKVREVGLTFQLKGAVPDLISANIENCMVEAFSQFKISDWNKLFWVVHPGGRAILDRVEAKLNLDPTKLIPTRHVMSEYGNMSSACVHFILDQTRKASLQNGCSTTGEGLEMGVLFGFGPGLTIETVVLKSVPIQ

SEQ ID NO: 32 - protein, 393 aa, Pinus densiflora
  misc_feature (1)..(393): Genbank BAA89667
MGGVDFEGFRKLQRADGFASILAIGTANPPNAVDQSTYPDYYFRITGNEHNTELKDKFKRICERSAIKQRYMYLTEEILKKNPDVCAFVEVPSLDARQAMLAMEVPRLAKEAAEKAIHEWGQSKSGITHLIFCSTTTPDLPGADFEVAKLLGLHPSVKRVGVFQHGCFAGGTVLRLAKDLAENNRGARVLVICSETTAVTFRGPSETHLDSLVGQALFGDGASALIVGADPIPQVEKACFEIVRTSQTVVPNSDGAIGGKVREVGLTFQLKGAVPDLISANIENCLVEAFSQFKICDWNKLFWVVHPGGRAILDRVEAKLNLDPTKLIPTRHVMSEYGNMSSACVHFILDETRKASLRNGCSTTGEGLEMGVLFGFGPGLTIETVVLKSVPLQ

SEQ ID NO: 33 - protein, 389 aa, Arachis hypogaea
  misc_feature (1)..(389): Genbank AAA96434.1
MVSVSGIRKVQRAEGPATVLAIGTANPPNCIDQSTYADYYFRVTNSEHMTDLKKKFQRICERTQIKNRHMYLTEEILKENPNMCAYKAPSLDAREDMMIREVPRVGKEAATKAIKEWGQPMSKITHLIFCTTSGVALPGVDYELIVLLGLDPCVKRYMMYHQGCFAGGTVLRLAKDLAENNKDARVLIVCSENTAVTFRGPSETDMDSLVGQALFADGAAAIIIGSDPVPEVEKPIFELVSTDQKLVPGSHGAIGGLLREVGLTFYLNKSVPDIISQNINDALNKAFDPLGISDYNSIFWIAHPGGRAILDQVEQKVNLKPEKMKATRDVLSNYGNMSSACVFFIMDLMRKRSLEEGLKTTGEGLDWGVLFGFGPGLTIETVVLRSVAI

SEQ ID NO: 34 - protein, 392 aa, Vitis quinquangularis
  misc_feature (1)..(392): Genbank AEZ00059.1
MASVEEIRNAQRAKGPATILAIGTATPDHCVYQSDYADYYFRVTKSEHMTELKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLETSVRRVMLYHQGCYAGGTVLRTAKDLAENNAGARVLVVCSEITVVTFRGPSEDALDSLVGQALFGDGSAAVIVGSDPDVSIERPLFQLVSAAQTFIPNSAGAIAGNLREVGLTFHLWPNVPTLISENIEKCLTQAFDPLGISDWNSLFWIAHPGGPAILDAVEAKLNLDKKKLEATRHVLSEYGNMSSACVLFILDEMRRKSLKGENGTTGEGLDWGVLFGFGPGLTIETVVLHSIPMITN

SEQ ID NO: 35 - protein, 392 aa, Vitis vinifera
  misc_feature (1)..(392): Genbank ABV82966.1
MASVEEIRNAQRAKGPATILAIGTATPDHCVYQSDYADYYFRVTKSEHMSELKKKFNRICDKSMIKKRYIHLTEEMLEEHPNIGAYMAPSLNIRQEIITAEVPKLGKEAALKALKEWGQPKSKITHLVFCTASGVEMPGADYKLANLLGLETSVRRVMLYHQGCYAGGTVLRTAKDLAENNAGARVLVVCSEITVVTFRGPSEDALDSLVGQALFGDGSAAVIVGSDPDVSIERPLFQLVSAAQTFIPNSAGAIAGNLREVGLTFHLWPNVPTLISENVEKCLTQAFDPLGISDWNSLFWIAHPGGPAILDAVEAKLNLDKKKLEATRHVLSEYGNMSSACVLFILDEMRKKSHKGEKATTGEGLDWGVLFGFGPGLTIETVVLHSIPMVTN

SEQ ID NO: 36 - protein, 210 aa, Polygonum cuspidatum
  misc_feature (1)..(210): Genbank AFP97666.1
MAASTDEMTKALTAATVLAIGTANPPNCYYQADFPDFYFRATNSDHLTHLKHKFKRICEKSMIEKRYLQLTEDILKENPNIGAYEAPSLDVRHEIQVKGVAQLGKEAALKAMQEWGQPKSKITHLIVCCIAGVDMPGANYQLTKLLDLNSSVKRFMFYHLGCYAGGTVLRLAKDIAENNKGARVLIVCSEMTPICFRGPSETHIDSMVGQ

SEQ ID NO: 37 - protein, 400 aa, Psilotum nudum
  misc_feature (1)..(400): Genbank BAA87924.1
MVSGEANGTSYQVRRGQSADGPATVLAIGTANPPNVFEQNSYPDFYFNVTNNTHKDELKAKFQRMCNRSGIRKRYLSFTEETLKANPSMGIYWEPSLDVRQDILAVEVPKLAKQASLNAIKEWGQPISNITHLVFCTTGPVSPGADAALMQMLGLNPSVKRVLLYMQGCFAGGTVLRHAKDLAENNKGARVLVVCSESTAVTFRGPHENHLDNLVGQALFADGAAALIVGSDPIPNVEKAWFEISLAESYLIPDSSPAIAGHLKEVGLEFHLTRDVSPVISKNILKILQDAFDGTGISDWNDVFIIAHPGGPAILDVIEEKLKLVPEKLQASRHVLSEFGNMSSATVHFILDHMRKSSVEKGCATTGEGYQLGILLGLGPGMTVESIVLKSVPIASLLSS

SEQ ID NO: 38 - protein, 399 aa, Morus notabilis
  misc_feature (1)..(399): Genbank AOA48577.1
MAPTNGFVEESQTVIPRAGPAVASILAIGTSNPSNYFNQAEYADYYFRVTNSEHMTELKEKFKRICEKSLIKKRHMRLTEDILKANPSICTYDGPSINERMDLKIVEMPKLGESAAIEALKEWGQPKSKITHIIVNSTSGVDMPGADYQLIRSLGLKTSVKRVMLYHQGCFAGGTVLRIAKDLAENNPGARVLVVCSELTIPTFRGPSEEDSASLVGQAIFADGGSAVIVGANVPDEGSVERPLFRLVSNSQVILPNSENTVGGHLRDCGLTIVLSPEVPKLIGKNILTCLEEAFTPFGISDWNSLFWVPHPGGAAILRAIEEKAELKKEKLKDTWNVWSEYGNMSSATVFFILNQMRKRSLEEKKSTTGDGLEWGVLLGFGPGLTVETVVLQSVPIVA

SEQ ID NO: 39 - protein, 399 aa, Morus notabilis
  misc_feature (1)..(399): Genbank AOA48578.1
MAPNNVSVEGSQPVIRRGGPGVASILAIGTANPDNFFNQADYPDYYFRVTNSEDKTELKEKFKRICEKSLIKKRHMRLTEDILKENPSMCSYDAPSLNARMDLKLVEMPKLGESAAIAAIKEWGQPKSKITHLIVNSTSGVDMPGADYQLIKSLGLERSVKRVMLYHQGCFAGGTVLRIAKDLAENNPGSRVLVVCSELTIPTFRGPSEDDSASLVGQAIFADGASAVIVGANVPDEGSVERPLFRLVSTSEVILPNSENTVGGHLRDCGLTIVLSPQVPKIIGKNIQTCLEEALGPFGISDWNSVFWAPHPGGAAIIKEIEDKAGLEKEKLKDTWNVWSEYGNMSSATVFFILNQMRKRSLEEKKSTTGDGLEWGVLLGFGPGLTVETVVLQSVPIVA

SEQ ID NO: 40 - protein, 403 aa, Psilotum nudum
  misc_feature (1)..(403): Genbank BAA87925.1
MTTGEASLMKNGPASRARREERADGPATELAIGTANPSNVFDQETYPDFYFDVTNNTDKPELKAKFQRMCNSLESKKVHVLHGGDVEGQPSMVFIGRILLDVRQGRRSRASAKLAKEASLKALREWGQPNSKITHLVFCTTAPVTLPGVDAALIQSLGLNPSVKRVLLYMQGCFAGGTVLRHAKDLAENNRGARVLVVCSETTAVTFRGPHENHLDNLVGQALFADGASALIVGSDPISDLEKPWFEIRWAGSYLIPESGQAIAGQLKEVGLEFHLTRDVSGLVSKNIVTILNEAFEGTGITDWNDIFIIPHPGGPAILDVIQDRLKLQPEKLQASRHVLAEFGNMSSATVHFSLDQMRRSSVEKGCSTTGEGYELGILLGLGPGMTVESILLKSVPTWTVAS

SEQ ID NO: 41 - protein, 390 aa, Malus domestica
  misc_feature (1)..(390): Genbank AFX71921.1
MAPLVKNHGEHQHAKILAIGTANPPNVYYQKDYPDFLFRVTKNEHRTDLREKFDRICEKSRTRKRYLHLTEEILKANPSIYTYGAPSLDVRQDMLNPEVPKLGQQAALKAIKEWGQPISKITHLIFCTASCVDMPGADFQLVKLLGLNPSVTRTMIYEAGCYAGATVLRLAKDFAENNEGARVLVVCAEITTVFFHGLTDTHLDILVGQALFADGASAVIVGANPEPKIERPLFEIVACRQTIIPNSEHGVVANIREMGFTYYLSGEVPKFVGGNVVDFLTKTFEKVDGKNKDWNSLFFSVHPGGPAIVDQVEEQLGLKEGKLRATRHVLSEYGNMGAPSVHFILDDMRKKSIEEGKSTTGEGLEWGVVIGIGPGLTVETAVLRSESITC

SEQ ID NO: 42 - protein, 388 aa, Malus domestica
  misc_feature (1)..(388): AFX71922.1
MAPLVKNEPQHAKILAIGTANPPNVYHQKDYPDFLFRVTKNEHRTDLREKFDRICEKSRTKKRYLHLTEEMLKANPNIYTYGAPSLDVRQDICNIEVPKLGQEAALKAIKEWGQPISRITHLIFCTASCVDMPGCDFQLIKLLGLDPSVTRTMIYEAGCYAGATVLRMAKDFAENNKGARVLVVCAEITTVFFHGLTDTHLDILVGQALFADGASAVIVGANPEPEIERPLFEIVACRQTILPNSEHGVVANIREMGFNYYLSGDVPKFVGGNVVDFMTKTFEKVDGKKKDWNSLFFSVHPGGPAIVDQVEEKLGLKEGKLRATRHVLSEYGNMGAPTVHFILDEMRNKSIEEGKTTTGEGLEWGVVIGIGPGLTVETAVLRSESIRC

SEQ ID NO: 43 - protein, 390 aa, Sorbus aucuparia
  misc_feature (1)..(390): ABB89212.1
MAPLVKNHGEPQHAKILAIGTANPPNVYYQKDYPDFLFRVTKNEHRTDLREKFDRICEKSRTRKRYLHLTEEILKANPSIYTYGAPSLDVRQDMLNSEVPKLGQQAALKAIKEWGQPISKITHLIFCTASCVDMPGADFQLVKLLGLNPSVTRTMIYEAGCYAGATVLRLAKDFAENNEGARVLVVCAEITTVFFHGLTDTHLDILVGQALFADGASAVIVGANPEPKIERPLFEIVACRQTIIPNSEHGVVANIREMGFTYYLSGEVPKFVGGNVVDFLTKTFEKVDGKNKDWNSLFFSVHPGGPAIVDQVEEQLGLKEGKLRATRHVLSEYGNMGAPSVHFILDDMRKKSIEEGKSTTGEGLEWGVVIGIGPGLTVETAVLRSESIPC

SEQ ID NO: 44 - protein, 390 aa, Pyrus communis
  misc_feature (1)..(390): ANT48448.1
MAPLVKNHVEPPHAKILAIGTANPPNVYYQKDYPDFLFRVTKNEHRTDLREKFDRICEKSRTRKRYLYLTEEILNANPSIYTYGAPSLDVRQDMLNPEVPKLGQQAALKAIKEWGQPISKITHLIFCTASCVDMPGADFQLVKLLGLNPSVTRTMIYEAGCYAGATVLRLAKDFAENNEDARVLVVCAEITTVFFHGLTDTHLDILVGQALFADGASAVIVGANPEPEIERPLFEIVACRQTIIPNSEHGVVANIREMGFNYYLSGDVPKFVGGSVVDFLTKTFEKVDGKNKDWNSLFLSVHPGGPAIVDQVEEQLGLKEGKLRATRHVLSEYGNMGAPSVHFILDEMRNKSIGEGKATTGEGLEWGVVIGIGPGLTVETAVLRSESIPC

SEQ ID NO: 45 - protein, 390 aa, Pyrus communis
  misc_feature (1)..(390): ANT48449.1
MAPLVKNHVEPPHAKILAIGTANPPNVYYQKDYPDFLFRVTKNEHRTDLREKFDRICEKSRTRKRYLYLTEEILNANPSIYTYGAPSLDVRQDMLNPEVPKLGQQAALKAIKEWGQPISKITHLIFCTASCVDMPGADFQLVKLLGLNPSVTRTMIYEAGCYAGATVLRLAKDFAENNEDARVLVVCAEITTVFFHGLTDTHLDILVGQALFADGASAVIVGANPEPEIESPLFEIVACRQTIIPNSEHGVVANIREMGFNYYLSGEVPKFVGGNVVDFLTKTFEKVDGKNKDWNSLFFSVHPGGPAIVDQVEEQLGLKEGKLRATRHVLSEYGNMGAPSVHFILDEMRKKSIEEGKATTGEGLEWGVVIGIGPGLTVETAVLRSESIPC

SEQ ID NO: 46 - protein, 390 aa, Phalaenopsis hybrid cultivar
  misc_feature (1)..(390): CAA56277.1
MPSLESIKKAPRADGFASILAIGRANPDNIIEQSAYPDFYFRVTNSEHLVDLKKKFQRICEKTAIRKRHFVWNEEFLTANPCFSTFMDKSLNVRQEVAISEIPKLGAKAATKAIEDWGQPKSRITHLIFCTTSGMDLPGADYQLTQILGLNPNVERVMLYQQGCFAGGTTLRLAKCLAESRKGARVLVVCAETTTVLFRAPSEEHQDDLVTQALFADGASAVIVGADPDEAADERASFVIVSTSQVLLPDSAGAIGGHVSEGGLLATLHRDVPQIVSKNVGKCLEEAFTPFGISDWNSIFWVPHPGGRAILDQVEERVGLKPEKLSVSRHVLAEYGNMSSVCVHFALDEMRKRSANEGKATTGEGLEWGVLFGFGPGLTVETVVLRSVPL

SEQ ID NO: 47 - protein, 390 aa, Phalaenopsis hybrid cultivar
  misc_feature (1)..(390): CAA56276.1
MLSLESIKKAPRADGFASILAIGRANPDNIIEQSAYPDFYFRVTNSEHLVDLKKKFQRICEKTAIRKRHFVWNEEFLTANPCFSTFMDKSLNVRQEVAISEIPKLGAKAATKAIEDWGQPKSRITHLIFCTTSGMDLPGADYQLTQILGLNPNVERVMLYQQGCFAGGTTLRLAKCLAESRKGARVLVVCAETTTVLFRAPSEEHQDDLVTQALFADGASAVIVGADPDEAADERASFVIVSTSQVLLPDSAGAIGGHVSEGGLLATLHRDVPQIVSKNVGKCLEEAFTPFGISDWNSIFWVPHPGGRAILDQVEERVGLKPEKLSVSRHVLAEYGNMSSVCVHFALDEMRKRSANEGKATTGEGLEWGVLFGFGPGLTVETVVLRSVPL

SEQ ID NO: 48 - protein, 394 aa, Bromheadia finlaysoniana; identical to SEQ ID NO: 12
  misc_feature (1)..(394): CAA10514.1

SEQ ID NO: 49 - protein, 388 aa, Dendrobium catenatum; identical to SEQ ID NO: 11
  misc_feature (1)..(388): PKU78204.1

SEQ ID NO: 50 - protein, 101 aa, Cannabis sativa
  misc_feature (1)..(101): Olivetolic Acid Cyclase (OAC) from Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 51 - protein, 305 aa, Streptomyces antibioticus
  misc_feature (1)..(305): Prenyltransferase from Streptomyces antibioticus
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 52 - protein, 545 aa, Cannabis sativa
  misc_feature (1)..(545): THCA synthase (THCAS) from Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 53 - protein, 390 aa, Malus domestica
  misc_feature (1)..(390): AEX88416.1 biphenyl synthase 2
MAPSVKDQVESQHAKILAIGTANPPNVYYQEDYPDFLFRVTKNEHRIDLREKFDRICEKSRTRKRYLYLTEEILKANPSIYTYGAPSLDVRQDMLNPEVPKLGQQAALKAIKEWGQPISKITHLIFCTASCVDMPGADFQLVKLLGLNPSVTRTMIYEAGCYAGATVLRLAKDFAENNEGARVLVVCAEITTVFFHGLTDTHLDILVGQALFADGASAVIVGANPEPEIESPLFEIVACRQTIIPNSEHGVVANIREMGFNYYLSGEVPKFVGGNVVDFLTKTFEKVDGKNKDWNSLFFSVHPGGPAIVDQVEEQLGLKEGKLRATRHVLSEYGNMGAPSVHFILDEMRKKSIEEGKATTGEGLEWGVVIGIGPGLTVETAVLRSEFITY

SEQ ID NO: 54 - protein, 394 aa, Malus domestica
  misc_feature (1)..(394): AEX88418.1 biphenyl synthase 4
MAPLVKNQVEPQHAKILAIGTANPPNVYYQEDYPDFLFRVTKNEHRTDLREKFDRICEKSRTKKRYLHVTEEILQANPSIYTYGAPSLDVRQDMLNPEVPKLGQQAALKAIKEWGQPISKITHLIFCTASCVDMPGADFQLVKLLGLNPSVTRTMIYEAGCYAGATVLRLAKDFAENNEGARVLVVCAEITTVFFHGLTDTHLDILVGQALFADGASAVIVGANPEPEIERPLFEIVACRQTIIPNSEHGVVAHIREMGFEYYLSGEVPKFVGGNVVDFLTKTFEKVDGKKKDWNSLFYSVHPGGPAIVDQVEEQLGLKEGKLRATRHVLSEYGNMGAPSVHFILDEMRNKSIEEGKSTTGEGLEWGVVIGIGPGLTVETIVLRSESIACEKLA

SEQ ID NO: 55 - protein, 389 aa, Arachis hypogaea
  misc_feature (1)..(389): AAU43217.1 chalcone synthase
MVNVSEIRKAQRAEGPATIMAIGTATPQNCVDQSTYPDYYFRTTNSQHMTELKEKFQRMCDKSMIKKRYMYLTEEILKENPNMCKYMAPSLDARQDMVVVEVPRLGKEAATKAIKEWGQPKSKITHLIFCTTSGVDMPGADYQLTKLLGLRPSVKRYMMYQQGCFAGGTVLRLAKDLAENNKGARVLVVCSEITAVTFRGPSETHLDSLVGQALFGDGAAALIVGSDPLPQIEKPVFELVWTAQTLAPDSEGAIDGHLREVGLTFHLLKDVPGIVSKNINKALTEAFDPLGISDYNSIFWIAHPGGPAILDQVEDKLKLKPHKLRATRDVLSDYGNMSSACVLFILDHMRNKSLQQGLQTTGEGLEWGVLFGFGPGLTIETVVLHSVAI

SEQ ID NO: 56 - protein, 398 aa, Sorghum bicolor
  misc_feature (1)..(398): AAL49965.1 chalcone synthase 8
MTTGKVTLEAVRKAQRAEGPATVLAIGTATPANCVYQADYPDYYFRVTKSEHLTDLKEKFKRICHKSMIRKRYMHLTEDILEENPNMSSYWAPSLDARQDILIQEIPKLGAEAAEKALKEWGQPRSRITHLVFCTTSGVDMPGADYQLIKLLGLCPSVNRAMMYHQGCFAGGMVLRLAKDLAENNRGARVLIVCSEITVVTFRGPSESHLDSLVGQALFGDGAAAVIVGADPSEPAERPLFHLVSASQTILPDSEGAIEGHLREVGLTFHLQDRVPQLISMNIERLLEDAFAPLGISDWNSIFWVAHPGGPAILNMVEAKVGLDKARMCATRHILAEYGNMSSVCVLFILDEMRNRSAKDGHTTTGEGMEWGVLFGFGPGLTVETIVLHSVPITTVAA

(19) United States
(12) Patent Application Publication
(10) Pub. No.: US 2024/0240160 A1
(43) Pub. Date: Jul. 18, 2024

(54) NOVEL OLIVETOL SYNTHASES FOR CANNABINOID PRODUCTION

(71) Applicant: GENOMATICA, INC., San Diego, CA (US)

(72) Inventors: Andreas W. SCHIRMER, San Diego, CA (US); Michael Angus NOBLE, San Diego, CA (US); Jamison Parker HUDDLESTON, San Diego, CA (US)

(21) Appl. No.: 18/564,967

(22) PCT Filed: May 27, 2022
(86) PCT No.: PCT/US2022/031361

§ 371 Date: Nov. 28, 2023

Related U.S. Application Data
(60) Provisional application No. 63/194,433, filed on May 28, 2021.

(51) Int. Cl. C12N 9/10; C12N 15/52; C12N 15/70; C12P 7/22; C12P 7/42; C12P 17/06; C12P 19/32

(52) U.S. Cl. CPC C12N 9/1029; C12N 15/52; C12N 15/70; C12P 7/22; C12P 7/42; C12P 17/06; C12P 19/32; C12Y 203/01206

(57) ABSTRACT

The present disclosure provides a poly nucleotide comprising: (a) a nucleic acid sequence encoding an olivetol synthase (OLS): and (b) a heterologous regulatory element operably linked to the nucleic acid sequence. The present disclosure further relates to an engineered cell comprising an olivetol synthase (OLS). Also provided are a cell extract or cell culture medium or a composition comprising 3.5.7- trioxododecanoyl-CoA, olivetol, olivetolic acid, a cannabinoid, and/or an isomer, analog, or derivative thereof, and a method of making 3.5.7-trioxododecanoyl-CoA, olivetol, olivetolic acid, a cannabinoid, and/or an isomer, analog, or derivative thereof. Also provided is a cannabinoid produced by the engineered cell, isolated from the cell extract or cell culture medium, and/or made by the method described herein. The present disclosure also provides a non-natural olivetol synthase (OLS).

### FIELD OF THE INVENTION

The present disclosure provides a polynucleotide comprising: (a) a nucleic acid sequence encoding an olivetol synthase (OLS) of any of SEQ ID NOs:2-49; and (b) a heterologous regulatory element operably linked to the nucleic acid sequence. The present disclosure further relates to an engineered cell comprising an olivetol synthase (OLS) of any of SEQ ID NOs:2-49. Also provided are a cell extract or cell culture medium or a composition comprising 3,5,7-trioxododecanoyl-CoA, olivetol, olivetolic acid, a cannabinoid, and/or an isomer, analog, or derivative thereof; a method of making 3,5,7-trioxododecanoyl-CoA, olivetol, olivetolic acid, a cannabinoid, and/or an isomer, analog, or derivative thereof. Also provided is a cannabinoid produced by the engineered cell, isolated from the cell extract or cell culture medium, and/or made by the method described herein. The present disclosure also provides a non-natural olivetol synthase (OLS) having at least 90% sequence identity to any of SEQ ID NOs:2-49 and comprising an amino acid substitution at an amino acid position corresponding to position 82, 125, 126, 131, 185, 186, 187, 189, 190, 195, 197, 204, 208, 209, 210, 211, 239, 249, 250, 257, 314, 331, and/or 332 of SEQ ID NO:1. The present disclosure further provides a non-naturally occurring olivetol synthase (OLS) comprising at least 90% sequence identity to any of SEQ ID NOs:2-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

### BACKGROUND

Cannabinoids constitute a varied class of chemicals, typically prenylated polyketides derived from fatty acid and isoprenoid precursors, that bind to cellular cannabinoid receptors. Modulation of these receptors has been associated with different types of physiological processes including pain-sensation, memory, mood, and appetite. Recently, cannabinoids have drawn significant scientific interest in their potential to treat a wide array of disorders, including insomnia, chronic pain, epilepsy, and post-traumatic stress disorder. See, e.g., Babson et al., Curr Psychiatry Rep 19:23 (2017); Romero-Sandoval et al., Curr Rheumatol Rep 19:67 (2017); O'Connell et al., Epilepsy Behav 70:341-348 (2017); and Zir-Aviv et al., Behav Pharmacol 27:561-569 (2016). Cannabinoid research and development as therapeutic tools requires production in large quantities and at high purity. However, purifying individual cannabinoid compounds from (′. sativa can be time-consuming and costly, and it can be difficult to isolate a pure sample of a compound of interest. Thus, engineered cells can be a useful alternative for the production of a specific cannabinoid or cannabinoid precursor.

### SUMMARY OF THE INVENTION

The present disclosure provides novel enzymes that produce cannabinoid precursors, e.g. olivetolic acid or precursors thereof. In some embodiments, the present disclosure provides novel olivetol synthases.

In some embodiments, the disclosure provides a polynucleotide comprising: (a) a nucleic acid sequence encoding an olivetol synthase (OLS) of any of SEQ ID NOs:2-49; and (b) a heterologous regulatory element operably linked to the nucleic acid sequence.

In some embodiments, the nucleic acid sequence encodes an OLS of SEQ ID NO:2, 3, 4, 6, 7, 8, 9, 11, 13, 14, 15, or 20. In some embodiments, the nucleic acid sequence encodes an OLS of SEQ ID NO:4, 6, 8, 9, 11, 13, 15, or 20. In some embodiments, the nucleic acid sequence encodes an OLS of SEQ ID NO:4, 6, 8, 9, 11, or 15. In some embodiments, the nucleic acid sequence encodes an OLS of SEQ ID NO:2, 3, 6, or 8. In some embodiments, the nucleic acid sequence encodes an OLS of SEQ ID NO:6 or 8. In some embodiments, the nucleic acid sequence encodes an OLS of SEQ ID NO:2. In some embodiments, the nucleic acid sequence encodes an OLS of SEQ ID NO:6. In some embodiments, the OLS further comprises an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the amino acid variation is an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof. In some embodiments, the amino acid variation is an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6. In some embodiments, the heterologous regulatory element comprises an Escherichia coli promoter.

In some embodiments, the disclosure provides a non-naturally occurring olivetol synthase (OLS) comprising at least 90% sequence identity to any of SEQ ID NOs:2-49 and comprising an amino acid substitution at an amino acid position corresponding to position 82, 125, 126, 131, 185, 186, 187, 189, 190, 195, 197, 204, 208, 209, 210, 211, 239, 249, 250, 257, 314, 331, and/or 332 of SEQ ID NO:1.

In some embodiments, the disclosure provides a non-naturally occurring olivetol synthase (OLS) comprising at least 90% sequence identity to any of SEQ ID NOs: 10-45, SEQ ID NO:48, or SEQ ID NO:49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

In some embodiments, the disclosure provides a non-naturally occurring olivetol synthase (OLS) comprising at least 95% sequence identity to SEQ ID NO:2 or any of SEQ ID NO:4-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the OLS comprises at least 97% sequence identity to SEQ ID NO:2 or 6.

In some embodiments, the amino acid variation is an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof.

In some embodiments, the amino acid variation is an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

In some embodiments, the disclosure provides a non-naturally occurring olivetol synthase (OLS) comprising at least 90% sequence identity to any of SEQ ID NOs:2-49, and further comprising an amino acid variation at an amino acid position corresponding to amino acid position 133, 134, 192, 193, 194, 196, 198, 214, 216, 218, 259, 266, 267, 268, 338, 340, and/or 380 of SEQ ID NO:6. In some embodiments, the amino acid variation is an amino acid substitution, wherein the amino acid substitution comprises S133A, S133G, S133W, G134H, E192D, T193S, T194A, T194E, T194N, T194Q, T194S, V196C, F198L, L214M, A216G, G218A, V259Q, V259W, V259Y, A266P, T267I, T267V, T267W, T267Y, L268M, L268V, M338L, M338T, S340A, V380L, or a combination thereof, wherein the amino acid position corresponds to SEQ ID NO:6. In some embodiments, the OLS comprises at least 90% sequence identity to SEQ ID NO:2 or 6. In some embodiments, the OLS comprises at least 97% sequence identity to SEQ ID NO:2 or 6.

In some embodiments, the disclosure provides a non-naturally occurring olivetol synthase (OLS) comprising at least 90% sequence identity to any of SEQ ID NOs:2-49, and further comprising an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof. In some embodiments, the OLS comprises at least 90% sequence identity to SEQ ID NO:2 or 6. In some embodiments, the OLS comprises at least 97% sequence identity to SEQ ID NO:2 or 6.

In some embodiments, the OLS produces at least 1.1-fold higher amount of olivetol and/or divarinol as compared to a wild-type counterpart of the OLS under the same reaction conditions. In some embodiments, a ratio of olivetol to pentyl diacetic acid lactone (OL:PDAL) production or a ratio of divarinol to propyl diacetic acid lactone (DVL:Propyl-DAL) production for the OLS is about 1.3-fold higher as compared to a wild-type counterpart of the OLS under the same reaction conditions.

In some embodiments, the disclosure provides a polynucleotide comprising a nucleic acid encoding the non-naturally occurring OLS described herein. In some embodiments, the polynucleotide comprises a heterologous regulatory element operably linked to the nucleic acid.

In some embodiments, the disclosure provides an expression construct comprising the polynucleotide described herein. In some embodiments, the expression construct is a bacterial expression construct.

In some embodiments, the disclosure provides an engineered cell comprising an olivetol synthase (OLS) of any of SEQ ID NOs:2-49. In some embodiments, the OLS comprises any of SEQ ID NOs:2, 3, 4, 6, 7, 8, 9, 11, 13, 14, 15, or 20. In some embodiments, the OLS comprises any of SEQ ID NOs: 4, 6, 8, 9, 11, 13, 15, or 20. In some embodiments, the OLS comprises any of SEQ ID NOs:2, 3, 6, or 8. In some embodiments, the OLS comprises any of SEQ ID NOs:6 or 8. In some embodiments, the OLS comprises SEQ ID NO:2. In some embodiments, the OLS comprises SEQ ID NO:6. In some embodiments, the OLS comprises an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

In some embodiments, the disclosure provides an engineered cell comprising a non-naturally occurring olivetol synthase (OLS), wherein the OLS comprises at least 90% sequence identity to any of SEQ ID NOs: 10-45, SEQ ID NO:48, or SEQ ID NO:49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

In some embodiments, the disclosure provides an engineered cell comprising a non-naturally occurring olivetol synthase (OLS) comprising at least 95% sequence identity to SEQ ID NO:2 or any of SEQ ID NO:4-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the OLS comprises at least 97% sequence identity to SEQ ID NO:2 or 6.

In some embodiments, the amino acid variation in the OLS is an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

In some embodiments, the amino acid variation in the OLS is an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

In some embodiments, the disclosure provides an engineered cell comprising a non-naturally occurring olivetol synthase (OLS) comprising at least 90% sequence identity to any of SEQ ID NOs:2-49, and further comprising an amino acid variation at an amino acid position corresponding to amino acid position 133, 134, 192, 193, 194, 196, 198, 214, 216, 218, 259, 266, 267, 268, 338, 340, and/or 380 of SEQ ID NO:6. In some embodiments, the amino acid variation is an amino acid substitution, wherein the amino acid substitution comprises S133A, S133G, S133W, G134H, E192D, T193S, T194A, T194E, T194N, T194Q, T194S, V196C, F198L, L214M, A216G, G218A, V259Q, V259W, V259Y, A266P, T267I, T267V, T267W, T267Y, L268M, L268V, M338L, M338T, S340A, V380L, or a combination thereof, wherein the amino acid position corresponds to SEQ ID NO:6. In some embodiments, the OLS comprises at least 90% sequence identity to SEQ ID NO:2 or 6. In some embodiments, the OLS comprises at least 97% sequence identity to SEQ ID NO:2 or 6.

In some embodiments, the disclosure provides an engineered cell comprising a non-naturally occurring olivetol synthase (OLS) comprising at least 90% sequence identity to any of SEQ ID NOs:2-49, and further comprising an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof. In some embodiments, the OLS comprises at least 90% sequence identity to SEQ ID NO:2 or 6. In some embodiments, the OLS comprises at least 97% sequence identity to SEQ ID NO:2 or 6.

In some embodiments, the disclosure provides an engineered cell comprising the polynucleotide described herein; the OLS described herein; and/or the expression construct described herein. In some embodiments, the cell comprises the polynucleotide, and the polynucleotide is integrated into a genome of the cell. In some embodiments, the cell comprises the polynucleotide, and the polynucleotide is present on an expression construct.

In some embodiments, the engineered cell further comprises a cannabinoid biosynthesis pathway enzyme and/or a polynucleotide encoding a cannabinoid biosynthesis pathway enzyme. In some embodiments, the cannabinoid biosynthesis pathway enzyme comprises olivetolic acid cyclase (OAC), prenyltransferase, a cannabinoid synthase, a geranyl pyrophosphate (GPP) biosynthesis pathway enzyme, or combination thereof.

In some embodiments, the OAC comprises an amino acid substitution at amino acid position H5, I7, L9, F23, F24, Y27, V46, T47, Q48, K49, N50, K51, V59, V61, V66, E67, 169, Q70, 173, 174, V79, G80, F81, G82, D83, R86, W89, L92, 194, D96, or a combination thereof, wherein the amino acid position is relative to SEQ ID NO:50. In some embodiments, the prenyltransferase comprises an amino acid substitution at amino acid position V45, V47, S49, F121, T124, Q159, M160, Y173, S212, V213, A230, 1232, T267, V269, Y286, T290, Q293, R294, L296, F300, or a combination thereof, wherein the amino acid position is relative to SEQ ID NO:51.

In some embodiments, the cannabinoid synthase comprises tetrahydrocannabinolic acid synthase (THCAS), cannabidiolic acid synthase (CBDAS), cannabichromenic acid synthase (CBCAS), or combination thereof. In some embodiments, the GPP biosynthesis pathway enzyme comprises geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, or a combination thereof.

In some embodiments, the cell is a bacterial cell. In some embodiments, the cell is an Escherichia coli cell.

In some embodiments, the cell is capable of producing 3,5,7-trioxododecanoyl-CoA, olivetol, olivetolic acid, a cannabinoid, an analog or derivative thereof; or a combination thereof. In some embodiments, the cell is further capable of producing olivetol; pentyl diacetic acid lactone (PDAL); hexanoyl triacetic acid lactone (HTAL); an analog or derivative thereof; or a combination thereof.

In some embodiments, the disclosure provides a cell extract or cell culture medium comprising 3,5,7-trioxododecanoyl-CoA, olivetol, olivetolic acid, a cannabinoid, and/or an analog or derivative thereof, wherein the cell culture extract or medium is derived from the engineered cell described herein.

In some embodiments, the disclosure provides a method of making 3,5,7-trioxododecanoyl-CoA, olivetol, olivetolic acid, a cannabinoid, and/or an analog or derivative thereof, comprising: culturing the engineered cell described herein; and/or isolating the 3,5,7-trioxododecanoyl-CoA, olivetol, olivetolic acid, cannabinoid, or analog or derivative thereof from the cell extract of cell culture medium described herein. In some embodiments, the engineered cell is cultured in the presence of hexanoic acid.

In some embodiments, the disclosure provides a composition comprising 3,5,7-trioxododecanoyl-CoA, olivetol, olivetolic acid, a cannabinoid, and/or an analog or derivative thereof, wherein the 3,5,7-trioxododecanoyl-CoA, olivetol, olivetolic acid, cannabinoid, and/or analog or derivative thereof is produced by the engineered cell described herein; isolated from the cell extract or cell culture medium described herein; and/or made by the method described herein.

In some embodiments, the composition comprises a cannabinoid selected from cannabigerolic acid (CBGA), tetrahydrocannabinolic acid (THCA), cannabidiolic acid (CBDA), cannabichromenic acid (CBCA), cannabigerol (CBG), tetrahydrocannabinol (THC), cannabidiol (CBD), cannabichromene (CBC), an analog or derivative thereof, or a combination thereof. In some embodiments, the composition is a therapeutic or medicinal composition, an oral unit dosage composition, a topical composition, or an edible composition.

In some embodiments, the disclosure provides a cannabinoid produced by the engineered cell described herein; isolated from the cell extract or cell culture medium described herein; and/or made by the method described herein. In some embodiments, the cannabinoid is cannabigerolic acid (CBGA), tetrahydrocannabinolic acid (THCA), cannabidiolic acid (CBDA), cannabichromenic acid (CBCA), cannabigerol (CBG), tetrahydrocannabinol (THC), cannabidiol (CBD), cannabichromene (CBC), an analog or derivative thereof, or a combination thereof.

In some embodiments, the disclosure provides a composition comprising: (a) the OLS described herein; and (b) hexanoyl-CoA, malonyl-CoA, 3,5,7-trioxododecanoyl-CoA, olivetol, PDAL, an analog, isomer, or derivative thereof, or a combination thereof.

### BRIEF DESCRIPTION OF THE DRAWINGS

The following drawings form part of the present specification and are included to further demonstrate exemplary embodiments of certain aspects of the present invention.

FIG. 1 shows an exemplary cannabinoid biosynthesis pathway as described in embodiments herein. Olivetol synthase (OLS) catalyzes the condensation of hexanoyl-CoA with three molecules of malonyl-CoA to yield 3,5,7-trioxododecanoyl-CoA, which is then converted to olivetolic acid by the enzyme olivetolic acid cyclase (OAC). A prenyltransferase converts olivetolic acid and geranyl pyrophosphate (GPP) to CBGA, which is then converted to tetrahydrocannabinolic acid (THCA) by THCA synthase (THCAS) or cannabidiolic acid (CBDA) by CBDA synthase. Hydrolytic byproducts of the OLS reaction are also shown.

FIG. 2 shows exemplary reactions catalyzed by three Type-III PKS enzymes. Olivetol synthase (OLS) catalyzes the conversion of hexanoyl-CoA to form 3,5,7-trioxododecanoyl-CoA, which is then converted to olivetolic acid and olivetol. Bibenzyl synthase (BBS) or biphenyl synthase (BIS) catalyzes the conversion of benzoyl-CoA to form the tetraketide precursor to 3,5-dihydroxybiphenyl. Stilbene synthase (STS) catalyzes the conversion of coumaroyl-CoA to form the tetraketide precursor to resveratrol.

FIG. 3 shows an exemplary specific activity assay of three Type-III PKS enzymes: QDX46968.1 (SEQ ID NO:6), AAZ32094.1 (SEQ ID NO:2), and QCO76957.1 (SEQ ID NO:8), and OLS from C. sativa with hexanoyl-CoA and malonyl-CoA, determined by formation of olivetolic acid (OLA) and pentyl diacetic acid lactone (PDAL).

FIG. 4 shows exemplary reactions catalyzed by OLS and olivetolic acid cyclase (OAC) with butyryl-CoA as the starter molecule, to form divarinic acid (DVA), divarinol (DVL), and propyl-diacetic acid lactone (propyl-DAL).

FIG. 5 shows an exemplary product inhibition assay of the OLS from C. sativa by olivetolic acid (OLA), as measured by enzyme activity on butyryl-CoA and monitoring formation of tetraketide products DVA and DVL and triketide product propyl-DAL.

FIG. 6 shows an exemplary product inhibition assay of the OLS from C. sativa by olivetol (OL), as measured by enzyme activity on butyryl-CoA and monitoring formation of tetraketide products DVA and DVL and triketide product propyl-DAL.

FIG. 7 shows an exemplary product inhibition assay of the OLS from (′. sativa by pentyl diacetic acid lactone (PDAL), as measured by enzyme activity on butyryl-CoA and monitoring formation of tetraketide products DVA and DVL and triketide product propyl-DAL.

FIG. 8 shows an exemplary product inhibition assay of the type-III PKS enzymes QDX46968.1 (SEQ ID NO:6) and AAZ32094.1 (SEQ ID NO:2) by OLA as measured by enzyme activity on butyryl-CoA and monitoring formation of tetraketide products DVA and DVL and triketide product propyl-DAL.

FIG. 9 shows an exemplary product inhibition assay of the type-III PKS enzymes QDX46968.1 (SEQ ID NO:6) and AAZ32094.1 (SEQ ID NO:2) by OL, as measured by enzyme activity on butyryl-CoA and monitoring formation of tetraketide products DVA and DVL and triketide product propyl-DAL.

FIG. 10 shows an exemplary product inhibition assay of the type-III PKS enzymes QDX46968.1 (SEQ ID NO:6) and AAZ32094.1 (SEQ ID NO:2) by pentyl diacetic acid lactone (PDAL), as measured by enzyme activity on butyryl-CoA and monitoring formation of tetraketide products DVA and DVL and triketide product propyl-DAL.

FIGS. 11-14 shows the results of exemplary activity assays with wild-type and variants of the OLS from Anoectochilus roxburghii (UniProt ID QDX46968.1; SEQ ID NO:6), designated as “OLS_Aro.”

FIG. 11 shows the fold-improvement in olivetol production by the variants of OLS_Aro over wild-type OLS_Aro.

FIG. 12 shows the fold-improvement in divarinol production by the variants of OLS_Aro over wild-type OLS_Aro.

FIG. 13 shows the fold-improvement in the OL/PDAL ratio of the variants of OLS_Aro over wild-type OLS_Aro.

FIG. 14 shows the fold-improvement in the DVL/Propyl-DAL ratio of the variants of OLS_Aro over wild-type OLS_Aro.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y.

A “nucleic acid,” “nucleic acid molecule,” “nucleic acid sequence,” “nucleotide sequence,” “oligonucleotide,” or “polynucleotide” means a polymeric compound including covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a nucleic acid encoding any one of the polypeptides disclosed herein, e.g., an OLS described herein.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “operably linked” means that a polynucleotide of interest, e.g., the polynucleotide encoding a nuclease, is linked to the regulatory element in a manner that allows for expression of the polynucleotide. In some embodiments, the regulatory element is a promoter. In some embodiments, a nucleic acid expressing the polypeptide of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Various promoters, including inducible promoters, may be used to drive expression of the various polynucleotides of the present disclosure. In some embodiments, the promoter comprises a bacterial promoter. In some embodiments, the promoter is an E. coli promoter.

An “expression vector” (also referred to as an “expression construct”) can be constructed to include one or more nucleic acids encoding one or more proteins of interest (e.g., nucleic acid encoding an OLS described herein) operably linked to expression control sequences functional in the host organism. Expression vectors applicable for use in the microbial host organisms provided include, for example, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli). In some embodiments, the expression vector comprises a nucleic acid encoding a protein described herein, e.g., an OLS. In some embodiments, the expression vector is suitable for expression a protein in a bacterial host cell, e.g., an E. coli cell.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like. When two or more exogenous encoding nucleic acids (e.g., gene encoding an OLS and an additional gene encoding another enzyme in a cannabinoid biosynthesis pathway such as, e.g., OAC, prenyltransferase, cannabinoid synthase, and/or an enzyme in the GPP pathway as described herein) are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein. The following vectors are provided by way of example; for bacterial host cells: pQE vector, pBluescript vector, pNH vector, lambda-ZAP vector, pTrc vector (e.g., pTrc99a), pTac vector, pUC vector, pDEST vector, pBAD vector, pET vector, p15 vector (e.g., p15a or p15b), pTD vector, pKK223 vector, pDR540 vector, pRIT2T vector. However, any other plasmid or vector may be used so long as it is compatible with the host cell.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.” In some embodiments, the present disclosure provides a host cell comprising an expression vector that comprises a nucleic acid encoding an OLS. In some embodiments, the host cell is a bacterial cell, a fungal cell, an algal cell, a cyanobacterial cell, or a plant cell. In some embodiments, the host cell is a bacterial cell. In some embodiments, the host cell is an E. coli cell.

A genetic alteration that makes an organism or cell non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

A host cell, organism, or microorganism engineered to express or overexpress a gene or a nucleic acid, or to overexpress an enzyme or polypeptide has been genetically engineered through recombinant DNA technology to include a gene or nucleic acid sequence that it does not naturally include, or to express an endogenous gene at a level that exceeds its level of expression in a non-altered cell. As non-limiting examples, a host cell, organism, or microorganism engineered to express or overexpress a gene or a nucleic acid, or to overexpress an enzyme or polypeptide can have any modifications that affect a coding sequence of a gene, the position of a gene on a chromosome or episome, or regulatory elements associated with a gene. A gene can also be overexpressed by increasing the copy number of a gene in the cell or organism. In some embodiments, overexpression of an endogenous gene comprises replacing the native promoter of the gene with a constitutive promoter that increases expression of the gene relative to expression in a control cell with the native promoter. In some embodiments, the constitutive promoter is heterologous.

Similarly, a host cell, organism, or microorganism engineered to under-express (or to have reduced expression of) a gene, nucleic acid, nucleic acid sequence, or nucleic acid molecule, or to under-express an enzyme or polypeptide, can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express or down-regulate a gene also include modifications to regulatory regions of the gene that can reduce its expression.

The term “exogenous” is intended to mean that the referenced molecule or the referenced activity is introduced into the host cell or host organism. The molecule can be introduced, for example, by introduction of an encoding nucleic acid into the host genetic material such as by integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a heterologous nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

Accordingly, the term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host microbial organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or a combination thereof, and still be considered as more than one exogenous nucleic acid. For example, as disclosed herein, a host cell or host organism can be engineered to express at least two, three, four, five, six, seven, eight, nine, ten or more exogenous nucleic acids encoding a desired pathway enzyme or protein. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, can be integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell host cell or host organism using techniques well known in the art including, but not limited to, conjugation, electroporation, chemical transformation, transduction, transfection, and ultrasound transformation. For exogenous expression in E. coli or other prokaryotic host cells, some nucleic acid sequences in the genes or cDNAs of eukaryotic nucleic acids can encode targeting signals such as an N-terminal mitochondrial or other targeting signal, which can be removed before transformation into the prokaryotic host cells, if desired. For example, removal of a mitochondrial leader sequence led to increased expression in E. coli (Hoffmeister et al. (2005), J Biol Chem 280: 4329-4338). For exogenous expression in yeast or other eukaryotic host cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon-optimized polynucleotides expressing an OLS.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (and also referred to as the amino-terminus, NH₂-terminus, or N-terminal end), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photo-cross-linked moieties, uniquely reactive functional groups, and NMR, IR, and x-ray crystallographic probes. Exemplary unnatural or synthetic amino acids are provided in, e.g., Mitra et al. (2013), Mater Methods 3:204 and Wals et al. (2014), Front Chem 2:15. Unnatural amino acids may also include naturally-occurring compounds that are not typically incorporated into a protein or polypeptide, such as, e.g., citrulline (Cit), selenocysteine (Sec), and pyrrolysine (Pyl).

As used herein, the terms “non-natural,” “non-naturally occurring,” “variant,” and “mutant” are used interchangeably in the context of an organism, polypeptide, or nucleic acid. The terms “non-natural,” “non-naturally occurring,” “variant,” and “mutant” in this context refer to a polypeptide or nucleic acid sequence having at least one variation or mutation at an amino acid position or nucleic acid position as compared to a wild-type polypeptide or nucleic acid sequence. The at least one variation can be, e.g., an insertion of one or more amino acids or nucleotides, a deletion of one or more amino acids or nucleotides, or a substitution of one or more amino acids or nucleotides. A “variant” protein or polypeptide is also referred to as a “non-natural” protein or polypeptide.

Naturally-occurring organisms, nucleic acids, and polypeptides can be referred to as “wild-type,” “wild type” or “original” or “natural” such as wild type strains of the referenced species, or a wild-type protein or nucleic acid sequence. Thus, a “wild-type counterpart” of a non-naturally occurring protein, e.g., OLS described herein, refers to a wild-type version of the referenced OLS as naturally found in the referenced species. Likewise, amino acids found in polypeptides of the wild type organism can be referred to as “original” or “natural” with regards to any amino acid position.

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic, unnatural, or naturally occurring amino acid. In some embodiments, the substituted amino acid is a naturally occurring amino acid as described herein. In some embodiments, the substituted amino acid is an unnatural or synthetic amino acid. Substitution mutants may be described using an abbreviated system. For example, a substitution mutation in which the fifth (5th) amino acid residue is substituted may be abbreviated as “X5Y,” wherein “X” is the wild-type amino acid to be replaced, “5” is the amino acid residue position within the amino acid sequence of the protein or polypeptide, and “Y” is the substituted amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

As used herein, the term “sequence similarity” (% similarity) refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: Positively-charged side chains: Arg, His, Lys; Negatively-charged side chains: Asp, Glu; Polar, uncharged side chains: Ser, Thr, Asn, Gln; Hydrophobic side chains: Ala, Val, Ile, Leu, Met, Phe, Tyr, Trp; Other: Cys, Gly, Pro.

In some embodiments, similar polypeptides of the present disclosure (e.g., OLS enzymes described herein) have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

The “percent identity” (% identity) between two polynucleotide or polypeptide sequences is determined when sequences are aligned for maximum homology, and generally not including gaps or truncations. Additional sequences added to a polypeptide sequence, such as but not limited to immunodetection tags, purification tags, localization sequences (presence or absence), etc., do not affect the % identity.

Algorithms known to those skilled in the art, such as Align, BLAST, ClustalW and others, compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest.

In some embodiments, similar polynucleotides of the present disclosure (e.g., encoding OLS enzymes described herein) have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical nucleic acid sequence. In some embodiments, similar polypeptides of the present disclosure (e.g., OLS enzymes described herein) have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acid sequence.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping, i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least at least 80%, at least 85%, at least 90%, at least 95%, at least 96%, at least 97% at least 98%, or at least 99% nucleotide or amino acid identity, respectively, to a reference sequence.

As used in the context of proteins, the term “structural similarity” indicates the degree of homology between the overall shape, fold, and/or topology of the proteins. It should be understood that two proteins do not necessarily need to have high sequence similarity to achieve structural similarity. Protein structural similarity is often measured by root mean squared deviation (RMSD), global distance test score (GDT-score), and template modeling score (TM-score); see, e.g., Xu and Zhang (2010), Bioinformatics 26(7):889-895. Structural similarity can be determined, e.g., by superimposing protein structures obtained from, e.g., x-ray crystallography, NMR spectroscopy, cryogenic electron microscopy (cryo-EM), mass spectrometry, or any combination thereof, and calculating the RMSD, GDT-score, and/or TM-score based on the superimposed structures. In some embodiments, two proteins have substantially similar tertiary structures when the TM-score is greater than about 0.5, greater than about 0.6, greater than about 0.7, greater than about 0.8, or greater than about 0.9. In some embodiments, two proteins have substantially identical tertiary structures when the TM-score is about 1.0. Structurally-similar proteins may also be identified computationally using algorithms such as, e.g., TM-align (Zhang et al., Nucleic Acids Res 33(7):2302-2309, 2005); DALI (Holm et al., J Mol Biol 233(1): 123-138, 1993); STRUCTAL (Gerstein et al., Proc Int Conf Intell Syst Mol Biol 4:59-69, 1996); MINRMS (Jewett et al., Bioinformatics 19(5):625-634, 2003); Combinatorial Extension (CE) (Shindyalov et al., Protein Eng 11(9): 739-747, 1998); ProtDex (Aung et al., DASFAA 2003, Proceedings); VAST (Gibrat et al., Curr Opin Struct Biol 6:377-385, 1996); LOCK (Singh et al., Proc Int Conf Intell Syst Mol Biol 5:284-293, 1997); and SSM (Krissinel et al., Acta Cryst D60:2256-2268, 2004).

### Olivetol Synthase

The present disclosure provides novel enzymes that produce cannabinoid precursors, e.g. olivetolic acid or precursors thereof. As used herein, “cannabinoid” refers to a prenylated polyketide or terpenophenolic compound derived from fatty acid or isoprenoid precursors. In general, cannabinoids are produced via a multi-step biosynthesis pathway, with the final precursor being a prenylated aromatic compound. In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG). In some embodiments, the prenylated aromatic compound is converted into a cannabinoid by oxidative cyclization. In some embodiments, CBGA is a precursor to tetrahydrocannabinolic acid (THCA), cannabidiolic acid (CBDA), and/or cannabichromenic acid (CBCA). Other prenylated aromatic compounds can be converted via analogous reactions into corresponding cannabinoids, e.g., THCOA, CBDOA, and CBCOA from CBGOA; THCVA, CBDVA, and CBCVA from CBGVA; THCO, CBDO, and CBCO from CBGO; THCV, CBDV, and CBCV from CBGV; and THC, CBD, and CBC from CBG. Further non-limiting examples of cannabinoids include, but are not limited to, cannabinolic acid (CBNA), cannabinol (CBN), cannabicyclol (CBL), cannabivarin (CBV), cannabielsoin (CBE), cannabicitran, and isomers, analogs or derivatives thereof. As used herein, an “isomer” of a reference compound has the same molecular formula as the reference compound, but with a different arrangement of the atoms in the molecule. As used herein, an “analog” or “structural analog” of a reference compound has a similar structure as the reference compound, but differs in a certain component such as an atom, a functional group, or a substructure. An analog can be imagined to be formed from the reference compound, but not necessarily formed or derived from the reference compound. As used herein, a “derivative” of a reference compound is derived from a similar compound by a similar reaction. Methods of identifying isomers, analogs or derivatives of the cannabinoids described herein are known to one of ordinary skill in the art.

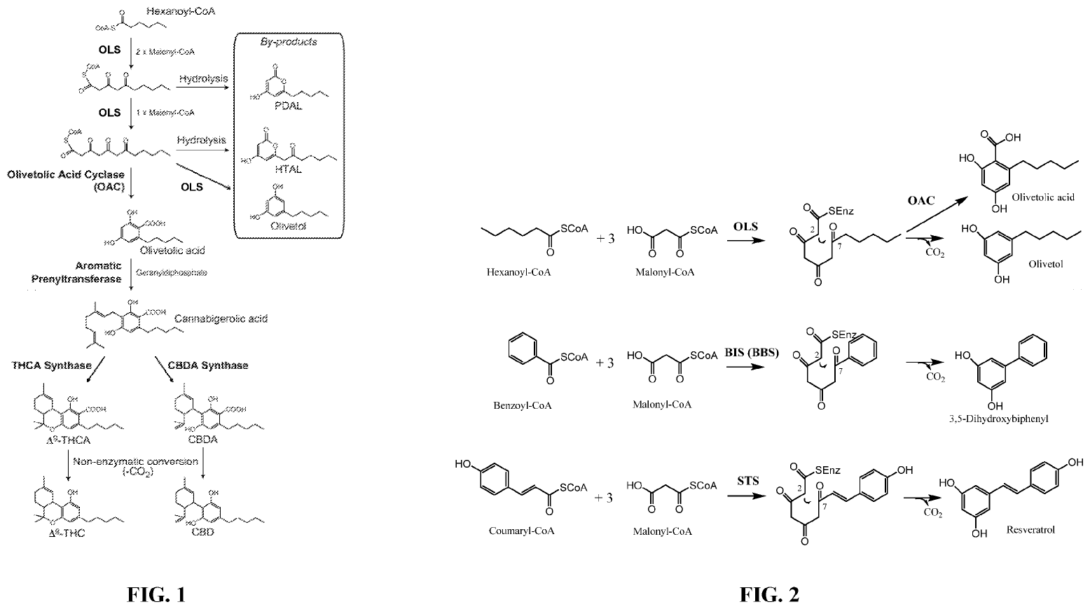

An exemplary cannabinoid biosynthesis pathway is illustrated in FIG. 1. As shown in FIG. 1, olivetol synthase (OLS) catalyzes the addition of two malonyl-CoA (Mal-CoA) and hexanoyl-CoA (Hex-CoA) to form a triketide (e.g., 3,5-dioxodecanoyl-CoA), which can be further converted by OLS to a tetraketide (e.g., 3,5,7-trioxododecanoyl-CoA) with the addition of a third Mal-CoA. As illustrated in FIG. 1, the triketide and tetraketide products produced by OLS can be hydrolyzed into various byproducts such as, e.g., pentyl diacetic lactone (PDAL), hexanoyl triacetic acid lactone (HTAL), or olivetol. In the cannabinoid biosynthesis pathway, the tetraketide product is subsequently converted to olivetolic acid by olivetolic acid cyclase (OAC). Olivetolic acid and geranyldiphosphate, also known as geranyl pyrophosphate or GPP, are condensed to form cannabigerolic acid (CBGA). CBGA can then be converted into various cannabinoids, e.g., tetrahydrocannabinolic acid (THCA) by THCA synthase or cannabidiolic acid (CBDA) by CBDA synthase, or cannabichromenic acid (CBCA) by CBCA synthase (not shown in FIG. 1).

Olivetol synthase (OLS) from Cannabis sativa belongs to the family of Type-III polyketide synthases (PKS).

In a general Type-III PKS enzyme reaction, a CoA-linked substrate compound is loaded onto an active site cysteine of the PKS and subjected to several rounds of carbon-carbon bond formation via decarboxylative Claisen condensation with malonyl-CoA as extender substrate to form an enzyme-bound polyketide compound. The polyketide compound can be then cyclized, most commonly via Claisen or aldol condensation and released from the PKS as a polyketide product, which can be further modified by tailoring enzymes. Type-III PKS are further described, e.g., in Morita et al., JBC Reviews 294:15121-15136 (2019).

For some type-III PKS enzyme reactions, the CoA-linked substrate is hexanoyl-, benzoyl-, or coumaroyl-CoA, and three rounds of carbon-carbon bond formation via decarboxylative Claisen condensation with malonyl-CoA as extender substrate are carried out to form a tetraketide compound. The tetraketide compound is then cyclized via a C2-C7 aldol condensation followed by decarboxylation to form a cyclic compound. An exemplary illustration of reactions performed by such type-III PKS is shown in FIG. 2: OLS, which acts upon hexanoyl-CoA to form the tetraketide precursor to olivetolic acid and olivetol; bibenzyl synthase (BBS) or biphenyl synthase (BIS), which acts upon benzoyl-CoA to form the tetraketide precursor to 3,5-dihydroxybiphenyl; and stilbene synthase (STS), which acts upon coumaroyl-CoA to form the tetraketide precursor to resveratrol. Table 1 shows an exemplary list of organisms and their BBS, BIS, and/or STS genes.

TABLE 1
Exemplary Organisms with Putative OLS Enzymes

| | UniProt or | SEQ |
|---|---|---|
| Organism | Genbank # | ID NO. |
| Cannabis sativa olivetol synthase | B1Q2B6 | 001 |
| Oncidium hybrid cultivar | AAZ32094.1 | 002 |
| Cymbidium hybrid cultivar | AIM58716.1 | 003 |
| Dendrobium catenatum | XP_020704098.1 | 004 |
| Phalaenopsis equestris | XP_020572025.1 | 005 |
| Anoectochilus roxburghii | QDX46968.1 | 006 |
| Phalaenopsis hybrid cultivar | AAX54693.1 | 007 |
| Dendrobium officinale | QCO76957.1 | 008 |
| Bletilla striata | AHH25569.1 | 009 |
| Oncidium Gower Ramsey | AAZ32093.1 | 010 |
| Dendrobium catenatum | XP_020704102.1 | 011 |
| Bromheadia finlaysoniana | CAA10514.1 | 012 |
| Paphiopedilum micranthum | AFU07718.1 | 013 |
| Paphiopedilum purpuratum | AFU07719.1 | 014 |
| Apostasia shenzhenica | PKA53998.1 | 015 |
| Paphiopedilum armeniacum | AFU07709.1 | 016 |
| Apostasia shenzhenica | PKA56805.1 | 017 |
| Paphiopedilum × areeanum | AFU07710.1 | 019 |
| Dendrobium catenatum | PKU71375.1 | 018 |
| Phalaenopsis equestris | XP_020591420.1 | 020 |
| Leersia perrieri | A0A0D9WZ61 | 021 |
| Leersia perrieri | A0A0D9W1R8 | 022 |
| Marchantia polymorpha | Q5I6Y1 | 023 |
| Marchantia polymorpha subsp. ruderalis | A0A176WB84 | 024 |
| Marchantia polymorpha | A0A2R6XE22 | 025 |
| Marchantia paleacea | A0A2P0VPI9 | 026 |
| Plagiochasma appendiculatum | AHY39238 | 027 |
| Plagiochasma appendiculatum | AIV42295.1 | 028 |
| Hydrangea macrophylla | AAN76182.1 | 029 |
| Hydrangea macrophylla | AAN76183.1 | 030 |
| Pinus sylvestris | Q02323.1 | 031 |
| Pinus densiflora | BAA89667.1 | 032 |
| Arachis hypogaea | AAA96434.1 | 033 |
| Vitis quinquangularis | AEZ00059.1 | 034 |
| Vitis vinifera | ABV82966.1 | 035 |
| Polygonum cuspidatum | AFP97666.1 | 036 |
| Psilotum nudum | BAA87924.1 | 037 |
| Morus notabilis | AOA48577.1 | 038 |
| Morus notabilis | AOA48578.1 | 039 |
| Psilotum nudum | BAA87925.1 | 040 |
| Malus domestica | AFX71921.1 | 041 |
| Malus domestica | AFX71922.1 | 042 |
| Sorbus aucuparia | ABB89212.1 | 043 |
| Pyrus communis | ANT48448.1 | 044 |
| Pyrus communis | ANT48449.1 | 045 |
| Phalaenopsis hybrid cultivar | CAA56277.1 | 046 |
| Phalaenopsis hybrid cultivar | CAA56276.1 | 047 |
| Bromheadia finlaysoniana | CAA10514.1 | 048 |
| Dendrobium catenatum | PKU78204.1 | 049 |
| Malus domestica | AEX88416.1 | 053 |
| Malus domestica | AEX88418.1 | 054 |
| Arachis hypogaea | AAU43217 | 055 |
| Sorghum bicolor | AAL49965 | 056 |

Type-III PKS can be promiscuous in their substrate usage. See, e.g., Lim et al., Molecules 21:806 (2016). Thus, Type-III PKS with relaxed specificity for their natural substrates (e.g., benzoyl-CoA for BBS or BIS; coumaroyl-CoA for STS) may produce olivetolic acid and/or olivetol in the presence of hexanoyl-CoA and olivetolic acid cyclase (OAC).

The present disclosure provides Type-III PKS enzymes that were not previously known to produce any cannabinoid precursors, e.g., olivetolic acid, in a host, e.g., a bacterial host. These Type-III PKS enzymes have relaxed substrate specificity and have olivetol synthase activity, i.e., producing 3,5,7-trioxododecanoyl-CoA from Hex-CoA. Thus, in some embodiments, the present disclosure provides novel OLS enzymes. The novel OLS enzymes described herein provide certain benefits as compared to the OLS from C. sativa (SEQ ID NO:1). For example, certain novel OLS enzymes of the present disclosure surprisingly provided higher levels of 3,5,7-trioxododecanoyl-CoA, the tetraketide precursor of olivetolic acid, as compared to the OLS from C. sativa. Moreover, the OLS from (′. sativa is feedback-inhibited by olivetol and olivetolic acid, which may limit the olivetolic acid titer in a heterologous host for cannabinoid production. In contrast, the novel OLS enzymes provided herein are not expected to be inhibited by olivetolic acid as olivetolic acid is an unnatural product for these novel OLS enzymes. In some embodiments, the novel OLS of the present disclosure produces higher amounts of the tetraketide precursor to olivetolic acid as compared to the OLS from (′. sativa under the same reaction conditions. In some embodiments, the novel OLS of the present disclosure, in combination with OAC, produces higher amounts of olivetolic acid as compared to the OLS from C. sativa in combination with OAC under the same reaction conditions. In some embodiments, the novel OLS of the present disclosure has higher enzymatic activity as compared to the OLS from (′. sativa. In some embodiments, the novel OLS of the present disclosure produces lower amounts of non-cannabinoid biosynthesis byproducts such as olivetol, PDAL, and/or HTAL as compared to the OLS from C. sativa.

In some embodiments, the present disclosure provides an OLS that is not substantially inhibited by a natural product of the OLS from C. sativa. In some embodiments, the OLS is not substantially inhibited by olivetolic acid, olivetol, pentyl diacetic acid lactone (PDAL), or combination thereof. In some embodiments, the OLS is not substantially inhibited by olivetolic acid or olivetol. As used herein, an enzyme activity that is “substantially not inhibited” by a particular compound means that the enzyme activity in the presence of the compound is at least 70%, at least 75%, at least 80%, at least 85%, at least 90%, at least 91%, at least 92%, at least 93%, at least 94%, at least 95%, at least 96%, at least 97%, at least 98%, at least 99%, at least 100%, at least 110%, at least 120%, at least 130%, at least 140%, or at least 150% of the enzyme activity in the absence of the compound. In some embodiments, the OLS has substantially the same enzyme activity in the presence or absence of olivetolic acid, olivetol, and/or PDAL.

In some embodiments, the OLS has a higher rate of production of olivetolic acid in the presence of hexanoyl-CoA, malonyl-CoA, and olivetolic acid cyclase (OAC) as compared to an OLS from C. sativa under the same reaction conditions. In some embodiments, the OLS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold higher rate of production of olivetolic acid in the presence of hexanoyl-CoA, malonyl-CoA, and OAC as compared to an OLS from C. sativa under the same reaction conditions. In some embodiments, the OLS has greater than about 1.1-fold, greater than about 1.2-fold, greater than about 1.3-fold, greater than about 1.4-fold, greater than about 1.5-fold, greater than about 1.6-fold, greater than about 1.7-fold, greater than about 1.8-fold, greater than about 1.9-fold, greater than about 2-fold, greater than about 2.5-fold, greater than about 3-fold, greater than about 4-fold, greater than about 5-fold, greater than about 6-fold, greater than about 7-fold, greater than about 8-fold, greater than about 9-fold, greater than about 10-fold, greater than about 15-fold, or greater than about 20-fold higher rate of production of olivetolic acid in the presence of hexanoyl-CoA, malonyl-CoA, and OAC as compared to an OLS from C. sativa under the same reaction conditions. Reaction conditions for production of olivetolic acid by OLS and OAC from hexanoyl-CoA and malonyl-CoA are described herein and known to one of skill in the art.

In some embodiments, the OLS, in combination with an OAC, has a higher rate of production of olivetolic acid in the presence of substrate (e.g., hexanoyl-CoA and malonyl-CoA), and product. As described herein, the novel OLS enzymes of the present disclosure are substantially not product-inhibited by the natural products of (′. sativa OLS (e.g., olivetolic acid, olivetol, and/or PDAL). In some embodiments, the OLS has a higher rate of production of olivetolic acid in the presence of hexanoyl-CoA; malonyl-CoA; OAC; and one or more of: olivetolic acid, olivetol, and/or PDAL as compared to an OLS from (′. sativa under the same reaction conditions. In some embodiments, the OLS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold higher rate of production of olivetolic acid in the presence of hexanoyl-CoA; malonyl-CoA; OAC; and one or more of: olivetolic acid, olivetol, and/or PDAL as compared to an OLS from (′. sativa under the same reaction conditions. In some embodiments, the OLS has greater than about 1.1-fold, greater than about 1.2-fold, greater than about 1.3-fold, greater than about 1.4-fold, greater than about 1.5-fold, greater than about 1.6-fold, greater than about 1.7-fold, greater than about 1.8-fold, greater than about 1.9-fold, greater than about 2-fold, greater than about 2.5-fold, greater than about 3-fold, greater than about 4-fold, greater than about 5-fold, greater than about 6-fold, greater than about 7-fold, greater than about 8-fold, greater than about 9-fold, greater than about 10-fold, greater than about 15-fold, or greater than about 20-fold rate of production of olivetolic acid in the presence of hexanoyl-CoA; malonyl-CoA; OAC; and one or more of: olivetolic acid, olivetol, and/or PDAL as compared to an OLS from C. sativa under the same reaction conditions.

In some embodiments, the present disclosure provides an OLS having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs:2-49. In some embodiments, the OLS has at least 70%, at least 80%, 90%, at least 95%, at least 99%, or 100% sequence identity to any of SEQ ID NOs:2, 3, 4, 6, 7, 8, 9, 11, 13, 14, 15, or 20. In some embodiments, the OLS has at least 70%, at least 80%, at least 90%, at least 95%, at least 99%, or 100% sequence identity to any of SEQ ID NOs:4, 6, 8, 9, 11, 13, 15, or 20. In some embodiments, the OLS has at least 70%, at least 80%, at least 90%, at least 95%, at least 99%, or 100% sequence identity to any of SEQ ID NOs:4, 6, 8, 9, 11, or 15. In some embodiments, the OLS has at least 70%, at least 80%, at least 90%, at least 95%, at least 99%, or 100% sequence identity to any of SEQ ID NOs:2, 3, 6, or 8. In some embodiments, the OLS has at least 70%, at least 80%, at least 90%, at least 95%, at least 99%, or 100% sequence identity to any of SEQ ID NOs:6 or 8. In some embodiments, the OLS is capable of producing 3,5,7-trioxododecanoyl-CoA from Hex-CoA. In some embodiments, the OLS is capable of producing 3,5,7-trioxododecanoyl-CoA, olivetol, PDAL, HTAL, an isomer, analog, or derivative thereof, or a combination thereof.

In some embodiments, the disclosure provides a non-natural OLS having 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs:2-49 and comprising at least one amino acid variation. As described herein, a “non-natural” or “non-naturally occurring” protein or polypeptide refers to a protein or polypeptide sequence having at least one amino acid variation as compared to a wild-type protein or polypeptide sequence. In some embodiments, the at least one amino acid variation comprises a substitution, deletion, insertion, or a combination thereof. In some embodiments, the at least one amino acid variation is not in an active site of the OLS. In some embodiments, the at least one amino acid variation is in an active site of the OLS. In some embodiments, the active site of the OLS comprises one or more amino acid residues involved in binding the substrate, cofactor, and/or coreactant, e.g., Hex-CoA or Mal-CoA. In some embodiments, an amino acid variation in the active site of the OLS improves binding of the OLS to the substrate, cofactor, and/or coreactant. In some embodiments, the active site of the OLS comprises one or more amino acid residues involved in catalysis, e.g., condensation of Hex-CoA and Mal-CoA. In some embodiments, an amino acid variation in the active site of the OLS improves reaction speed and/or efficiency of the catalysis. In some embodiments, the non-natural OLS is capable of producing 3,5,7-trioxododecanoyl-CoA from Hex-CoA. In some embodiments, the non-natural OLS is capable of producing 3,5,7-trioxododecanoyl-CoA, olivetol, PDAL, HTAL, an isomer, analog, or derivative thereof, or a combination thereof.

In some embodiments, the disclosure provides a non-naturally occurring OLS having at least 90% sequence identity to any of SEQ ID NOs:2-49 and comprising an amino acid substitution at an amino acid position corresponding to position 82, 125, 126, 131, 185, 186, 187, 189, 190, 195, 197, 204, 208, 209, 210, 211, 239, 249, 250, 257, 314, 331, and/or 332 of SEQ ID NO:1. In some embodiments, the non-natural OLS has at least 90% sequence identity to any of SEQ ID NOs:2, 3, 4, 6, 7, 8, 9, 11, 13, 14, 15, or 20. In some embodiments, the non-natural OLS has at least 90% sequence identity to any of SEQ ID NOs:4, 6, 8, 9, 11, 13, 15, or 20. In some embodiments, the non-natural OLS has at least 90% sequence identity to any of SEQ ID NOs:4, 6, 8, 9, 11, or 15. In some embodiments, the non-natural OLS has at least 90% sequence identity to any of SEQ ID NOs:2, 3, 6, or 8. In some embodiments, the non-natural OLS has at least 90% sequence identity to any of SEQ ID NOs:6 or 8. Non-natural OLS, e.g., comprising the amino acid substitutions described herein, are further described in, e.g., WO2020/214951. It will be understood by one of ordinary skill in the art that alignment methods can be used to determine the appropriate amino acid number that corresponds to the position referenced in SEQ ID NO:1 and/or SEQ ID NO:6 as described herein.

In some embodiments, the disclosure provides further non-naturally occurring OLS that have improved activity, e.g., improved yield of cannabinoid precursors, e.g., olivetol from Hex-CoA, and/or decreased reaction byproducts (such as PDAL and/or HTAL) as compared to a wild-type counterpart of the OLS. In some embodiments, the non-natural OLS produces at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold, at least 15-fold, or at least 20-fold higher amount of olivetol from Hex-CoA and/or divarinol from butyryl-CoA as compared to a wild-type counterpart of the non-natural OLS under the same reaction conditions.

In some embodiments, a ratio of the olivetol to PDAL (OL:PDAL) production from Hex-CoA; and/or a ratio of the divarinol to propyl diacetic acid lactone (DVL:Propyl-DAL) production from butyryl-CoA for the non-natural OLS is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold, at least 15-fold, or at least 20-fold higher as compared to a wild-type counterpart of the non-natural OLS under the same reaction conditions.

In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 60 to 80, or amino acid positions 65 to 75, or amino acid positions 68 to 72 of SEQ ID NO:6. In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 120 to 150, or amino acid positions 125 to 145, or amino acid positions 130 to 140 of SEQ ID NO:6. In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 150 to 170, or amino acid positions 155 to 165, or amino acid positions 158 to 163 of SEQ ID NO:6. In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 180 to 230, or amino acid positions 185 to 225, or amino acid positions 190 to 220 of SEQ ID NO:6. In some embodiments, the amino acid variation is an amino acid substitution.

In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 240 to 280, or amino acid positions 245 to 275, or amino acid positions 250 to 270 of SEQ ID NO:6. In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 290 to 320, or amino acid positions 295 to 315, or amino acid positions 300 to 310 of SEQ ID NO:6. In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 325 to 355, or amino acid positions 330 to 350, or amino acid positions 335 to 345 of SEQ ID NO:6. In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising one or more amino acid variations at an amino acid position in a region corresponding to amino acid positions 360 to 400, or amino acid positions 365 to 395, or amino acid positions 370 to 390 of SEQ ID NO:6. In some embodiments, the amino acid variation is an amino acid substitution.

In some embodiments, the disclosure provides a non-naturally occurring OLS comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:2-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the amino acid variation is an amino acid substitution. In some embodiments, the non-natural OLS comprises at least 90%, at least 91%, at least 92%, at least 93%, at least 94%, at least 95%, at least 96%, at least 97%, at least 98%, at least 99%, or 100% sequence identity to SEQ ID NO:2. In some embodiments, the non-natural OLS comprises at least 90%, at least 91%, at least 92%, at least 93%, at least 94%, at least 95%, at least 96%, at least 97%, at least 98%, at least 99%, or 100% sequence identity to SEQ ID NO:6.

In some embodiments, the disclosure provides a non-naturally occurring OLS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs: 10-45, SEQ ID NO:48, or SEQ ID NO:49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the non-natural OLS comprises at least 90%, at least 91%, at least 92%, at least 93%, at least 94%, at least 95%, at least 96%, at least 97%, at least 98%, at least 99%, or 100% sequence identity to any of SEQ ID NOs: 10-45, SEQ ID NO:48, or SEQ ID NO:49.

In some embodiments, the disclosure provides a non-naturally occurring OLS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:2 or any of SEQ ID NO:4-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the non-natural OLS comprises at least 95%, at least 96%, at least 97%, at least 98%, at least 99%, or 100% sequence identity to SEQ ID NO:2 or any of SEQ ID NO:4-49. In some embodiments, the non-natural OLS comprises at least 97%, at least 98%, at least 99%, or 100% sequence identity to SEQ ID NO:2. In some embodiments, the non-natural OLS comprises at least 97%, at least 98%, at least 99%, or 100% sequence identity to SEQ ID NO:6.

In some embodiments, the disclosure provides a non-naturally occurring OLS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs:2-49, and further comprising an amino acid variation at an amino acid position corresponding to amino acid position 133, 134, 192, 193, 194, 196, 198, 214, 216, 218, 259, 266, 267, 268, 338, 340, and/or 380 of SEQ ID NO:6. In some embodiments, the non-natural OLS comprises at least 90%, at least 91%, at least 92%, at least 93%, at least 94%, at least 95%, at least 96%, at least 97%, at least 98%, at least 99%, or 100% sequence identity to any of SEQ ID NOs:2-49. In some embodiments, the non-natural OLS comprises at least 90%, at least 91%, at least 92%, at least 93%, at least 94%, at least 95%, at least 96%, at least 97%, at least 98%, at least 99%, or 100% sequence identity to SEQ ID NO:2. In some embodiments, the non-natural OLS comprises at least 90%, at least 91%, at least 92%, at least 93%, at least 94%, at least 95%, at least 96%, at least 97%, at least 98%, at least 99%, or 100% sequence identity to SEQ ID NO:6.

In some embodiments, the amino acid variation in the non-natural OLS comprises an amino acid substitution. In some embodiments, the amino acid substitution at amino acid position 70 is F70N, F70Q, or F70V. In some embodiments, the amino acid substitution at amino acid position 70 is F70N or F70Q. In some embodiments, the amino acid substitution at amino acid position 70 is F70M. In some embodiments, the amino acid substitution at amino acid position 133 is S133A, S133G, or S133W. In some embodiments, the amino acid substitution at amino acid position 134 is G134H. In some embodiments, the amino acid substitution at amino acid position Y160 is Y160G. In some embodiments, the amino acid substitution at amino acid position Q161 is Q161H, Q161M, Q161T, Q161L, Q161Y, Q161W, Q161V, Q161G, or Q161F. In some embodiments, the amino acid substitution at amino acid position 161 is Q161H, Q161M, or Q161T.

In some embodiments, the amino acid substitution at amino acid position 192 is E192D. In some embodiments, the amino acid substitution at amino acid position 193 is T193S. In some embodiments, the amino acid substitution at amino acid position 194 is T194A, T194E, T194N, T194Q, or T194S. In some embodiments, the amino acid substitution at amino acid position 195 is T195M. In some embodiments, the amino acid substitution at amino acid position 196 is V196C. In some embodiments, the amino acid substitution at amino acid position 198 is F198L. In some embodiments, the amino acid substitution at amino acid position 207 is E207C. In some embodiments, the amino acid substitution at amino acid position 208 is D208H. In some embodiments, the amino acid substitution at amino acid position 214 is L214M. In some embodiments, the amino acid substitution at amino acid position 216 is A216G. In some embodiments, the amino acid substitution at amino acid position 218 is G218A. In some embodiments, the amino acid substitution at amino acid position 255 is I255L, I255S, and I255M. In some embodiments, the amino acid substitution at amino acid position 255 is I255L.

In some embodiments, the amino acid substitution at amino acid position 259 is V259Q, V259W, or V259Y. In some embodiments, the amino acid substitution at amino acid position 264 is L264F. In some embodiments, the amino acid substitution at amino acid position 266 is A266P. In some embodiments, the amino acid substitution at amino acid position 267 is T267I, T267V, T267W, or T267Y. In some embodiments, the amino acid substitution at amino acid position 268 is L268M or L268V. In some embodiments, the amino acid substitution at amino acid position 269 is H269T. In some embodiments, the amino acid substitution at amino acid position 303 is P303A, P303C, P303I, P303L, P303M, P303T, or P303V. In some embodiments, the amino acid substitution at amino acid position 305 is P305L. In some embodiments, the amino acid substitution at amino acid position 338 is M338L or M338T. In some embodiments, the amino acid substitution at amino acid position 339 is S339W. In some embodiments, the amino acid substitution at amino acid position 340 is S340A. In some embodiments, the amino acid substitution at amino acid position 373 is G373A. In some embodiments, the amino acid substitution at amino acid position 374 is F374I, F374M, or F374V. In some embodiments, the amino acid substitution at amino acid position 380 is V380L. Unless otherwise specified, the amino acid positions correspond to SEQ ID NO:6.

In some embodiments, the amino acid variation in the non-natural OLS comprises an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

In some embodiments, the amino acid variation in the non-natural OLS comprises an amino acid substitution, wherein the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

In some embodiments, the amino acid variation in the non-natural OLS comprises an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

In some embodiments, the amino acid variation in the non-natural OLS comprises an amino acid substitution, wherein the amino acid substitution comprises S133A, S133G, S133W, G134H, E192D, T193S, T194A, T194E, T194N, T194Q, T194S, V196C, F198L, L214M, A216G, G218A, V259Q, V259W, V259Y, A266P, T267I, T267V, T267W, T267Y, L268M, L268V, M338L, M338T, S340A, V380L, or a combination thereof, wherein the amino acid position corresponds to SEQ ID NO:6.

In some embodiments, the disclosure provides a non-naturally occurring OLS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs:2-49, and further comprising an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the non-natural OLS comprises at least 90%, at least 91%, at least 92%, at least 93%, at least 94%, at least 95%, at least 96%, at least 97%, at least 98%, at least 99%, or 100% sequence identity to any of SEQ ID NOs:2-49. In some embodiments, the non-natural OLS comprises at least 90%, at least 91%, at least 92%, at least 93%, at least 94%, at least 95%, at least 96%, at least 97%, at least 98%, at least 99%, or 100% sequence identity to SEQ ID NO:2. In some embodiments, the non-natural OLS comprises at least 90%, at least 91%, at least 92%, at least 93%, at least 94%, at least 95%, at least 96%, at least 97%, at least 98%, at least 99%, or 100% sequence identity to SEQ ID NO:6.

In some embodiments, the OLS of any of SEQ ID NOs:2-49 and/or the non-natural OLS described herein has substantial structural similarity to the OLS from C. sativa (SEQ ID NO:1). In some embodiments, the OLS comprises a structurally similar active site as the OLS from C. sativa. In some embodiments, the OLS is capable of using Hex-CoA as a substrate. In some embodiments, the OLS is capable of producing 3,5,7-trioxododecanoyl-CoA from Hex-CoA. In some embodiments, the OLS is capable of producing 3,5,7-trioxododecanoyl-CoA, olivetol, PDAL, HTAL, an isomer, analog, or derivative thereof, or a combination thereof. In some embodiments, the OLS is capable of using a Hex-CoA analog as a substrate. Hex-CoA analogs that may be used as OLS substrate include, for example and without limitation, acetyl-CoA, propionyl-CoA, butyryl-CoA, pentanoyl-CoA, heptanoyl-CoA, octanoyl-CoA, nonanoyl-CoA, decanoyl-CoA, any C2-C20 acyl-CoA, and/or an aromatic acid CoA, e.g., benzoic, chorismic, phenylacetic, and phenoxyacetic acid-CoA. It will be understood by one of ordinary skill in the art that, when a Hex-CoA analog is used as substrate for the OLS described herein, analogous product(s) are produced. For example, the OLS is capable of producing olivetol from Hex-CoA and is further capable of producing divarinol from butyryl-CoA. In an analogous manner, the reaction byproducts from butyryl-CoA comprise propyl-diacetic acid lactone (Propyl-DAL).

In some embodiments, the disclosure provides a polynucleotide encoding the non-natural OLS described herein. In some embodiments, the polynucleotide further comprises a heterologous bacterial regulatory element operably linked to the nucleic acid sequence. In some embodiments, the disclosure provides a polynucleotide comprising: (a) a nucleic acid sequence encoding an olivetol synthase (OLS) of any of SEQ ID NOs:2-49; and (b) a heterologous regulatory element operably linked to the nucleic acid sequence, e.g., a bacterial regulatory element.

In some embodiments, the nucleic acid encodes an OLS having at least 70%, at least 80%, at least 90%, at least 95%, or at least 99% sequence identity to any of SEQ ID NOs:2, 3, 4, 6, 7, 8, 9, 11, 13, 14, 15, or 20. In some embodiments, the nucleic acid encodes an OLS of SEQ ID NO:2, 3, 4, 6, 7, 8, 9, 11, 13, 14, 15, or 20.

In some embodiments, the nucleic acid encodes an OLS having at least 70%, at least 80%, at least 90%, at least 95%, or at least 99% sequence identity to any of SEQ ID NOs:4, 6, 8, 9, 11, 13, 15, or 20. In some embodiments, the nucleic acid sequence encodes an OLS of SEQ ID NO:4, 6, 8, 9, 11, 13, 15, or 20.

In some embodiments, the nucleic acid encodes an OLS having at least 70%, at least 80%, at least 90%, at least 95%, or at least 99% sequence identity to any of SEQ ID NOs:4, 6, 8, 9, 11, or 15. In some embodiments, the nucleic acid sequence encodes an OLS of SEQ ID NO:4, 6, 8, 9, 11, or 15.

In some embodiments, the nucleic acid encodes an OLS having at least 70%, at least 80%, at least 90%, at least 95%, or at least 99% sequence identity to any of SEQ ID NOs:2, 3, 6, or 8. In some embodiments, the nucleic acid sequence encodes an OLS of SEQ ID NO:2, 3, 6, or 8.

In some embodiments, the nucleic acid encodes an OLS having at least 70%, at least 80%, at least 90%, at least 95%, or at least 99% sequence identity to any of SEQ ID NOs:6 or 8. In some embodiments, the nucleic acid sequence encodes an OLS of SEQ ID NO:6 or 8.

In some embodiments, the nucleic acid encodes an OLS having at least 70%, at least 80%, at least 90%, at least 95%, or at least 99% sequence identity to any of SEQ ID NO:2. In some embodiments, the nucleic acid sequence encodes an OLS of SEQ ID NO:2.

In some embodiments, the nucleic acid encodes an OLS having at least 70%, at least 80%, at least 90%, at least 95%, or at least 99% sequence identity to any of SEQ ID NO:6. In some embodiments, the nucleic acid sequence encodes an OLS of SEQ ID NO:6.

In some embodiments, the nucleic acid encodes an OLS comprising at least 70%, at least 80%, at least 90%, at least 95%, or at least 99% sequence identity to any one of SEQ ID NOs:2-49 and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the nucleic acid encodes an OLS comprising at least 90% sequence identity to SEQ ID NO:2 or 6 and further comprising the amino acid variation as described herein. In some embodiments, the amino acid variation is an amino acid substitution. In some embodiments, the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

In some embodiments, the OLS encoded by the nucleic acid has substantial structural similarity to the OLS from C. sativa (SEQ ID NO:1). In some embodiments, the OLS comprises a structurally similar active site as the OLS from C. sativa. In some embodiments, the OLS is capable of using Hex-CoA as a substrate. In some embodiments, the OLS is capable of using a Hex-CoA analog as a substrate. Hex-CoA analogs are further described herein.

In some embodiments, the OLS encoded by the nucleic acid is capable of producing the 3,5,7-trioxododecanoyl-CoA from Hex-CoA. In some embodiments, the OLS encoded by the nucleic acid is capable of producing 3,5,7-trioxododecanoyl-CoA, olivetol, PDAL, HTAL, an isomer, analog, or derivative thereof, or a combination thereof.

In some embodiments, the heterologous regulatory element, e.g., a bacterial regulatory element, of the polynucleotide comprises a promoter, an enhancer, a silencer, a response element, or a combination thereof. As used herein, a “bacterial regulatory element” refers to a regulatory element that is derived from a bacterial genome (i.e., a bacterial genomic promoter), or a regulatory element that regulates bacterial plasmid expression (i.e., a bacteria plasmid promoter). Non-limiting examples of bacterial regulatory elements include bacterial promoters such as the σ70 promoter, σS promoter, σ32 promoter, and σ54 promoter; and bacterial plasmid promoters such as the T7 promoter, T5 promoter, Tac promoter, araBad promoter, Trc promoter, lac promoter, PrpB promoter, Tet promoter, Sp6 promoter, and Trp promoter. In some embodiments, the bacterial regulatory element is an inducible promoter. In some embodiments, the inducible promoter is a tetracycline-regulated promoter, a steroid-regulated promoter, a metal-regulated promoter, a pathogenesis-regulated promoter, a temperature/heat-inducible promoter, a light-inducible promoter, a galactose-inducible promoter, or combination thereof. In some embodiments, the heterologous bacterial regulatory element comprises an Escherichia coli promoter.

In some embodiments, the disclosure provides an expression construct comprising the polynucleotide described herein. In some embodiments, the expression construct is a bacterial expression construct. Expression constructs are further described herein. In some embodiments, the expression construct comprises a pQE vector, a pBluescript vector, a pNH vector, a lambda-ZAP vector, a pTrc vector (e.g., pTrc99a), a pTac vector, a pUC vector, a pDEST vector, a pBAD vector, a pET vector, a p15 vector (e.g., p15a or p15b), a pTD vector, a pKK223 vector, a pDR540 vector, a pRIT2T vector, or a combination thereof. In some embodiments, the expression construct comprises a bacterial regulatory element, e.g., a bacterial genomic promoter or a bacterial plasmid promoter. Bacterial regulatory elements are further described herein.

In some embodiments, the disclosure provides an olivetol synthase (OLS) encoded by the polynucleotide described herein. In some embodiments, the OLS is not substantially inhibited by a natural product of the OLS from (′. sativa. In some embodiments, the OLS is not substantially inhibited by olivetolic acid, olivetol, pentyl diacetic acid lactone (PDAL), or combination thereof. In some embodiments, the OLS is not substantially inhibited by olivetolic acid or olivetol. In some embodiments, the OLS has substantially the same enzyme activity in the presence or absence of olivetolic acid, olivetol, and/or PDAL.

In some embodiments, the OLS encoded by the polynucleotide described herein has a higher rate of production of olivetolic acid in the presence of hexanoyl-CoA, malonyl-CoA, and olivetolic acid cyclase (OAC) as compared to an OLS from C. sativa under the same reaction conditions. In some embodiments, the OLS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, or 20-fold higher rate of production of olivetolic acid in the presence of hexanoyl-CoA, malonyl-CoA, and OAC as compared to an OLS from C. sativa under the same reaction conditions.

In some embodiments, the OLS encoded by the polynucleotide described herein has a higher rate of production of olivetolic acid in the presence of hexanoyl-CoA; malonyl-CoA; OAC; and one or more of: olivetolic acid, olivetol, and/or PDAL as compared to an OLS from (′. sativa under the same reaction conditions. In some embodiments, the OLS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, or more than 20-fold higher rate of production of olivetolic acid in the presence of hexanoyl-CoA; malonyl-CoA; OAC; and one or more of: olivetolic acid, olivetol, and/or PDAL as compared to an OLS from C. sativa under the same reaction conditions.

In some embodiments, the OLS encoded by the polynucleotide described herein is a non-natural OLS. In some embodiments, the non-natural OLS produces at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold, at least 15-fold, or at least 20-fold higher amount of olivetol from Hex-CoA and/or divarinol from butyryl-CoA as compared to a wild-type counterpart of the non-natural OLS under the same reaction conditions.

In some embodiments, the non-natural OLS encoded by the polynucleotide described herein provides a ratio of the olivetol to PDAL (OL:PDAL) production from Hex-CoA; and/or a ratio of the divarinol to propyl diacetic acid lactone (DVL:Propyl-DAL) production from butyryl-CoA that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold, at least 15-fold, or at least 20-fold higher as compared to a wild-type counterpart of the non-natural OLS under the same reaction conditions.

### Engineered Cell

In some embodiments, the present disclosure further provides methods for production of cannabinoids and cannabinoid precursors using engineered cells. In some embodiments, the disclosure provides an engineered cell comprising the OLS described herein, e.g., having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs:2-49. In some embodiments, the disclosure provides an engineered cell comprising the non-natural OLS described herein, e.g., having at least 90% sequence identity to any of SEQ ID NOs:2-49 and comprising an amino acid substitution at an amino acid position corresponding to position 82, 125, 126, 131, 185, 186, 187, 189, 190, 195, 197, 204, 208, 209, 210, 211, 239, 249, 250, 257, 314, 331, and/or 332 of SEQ ID NO:1. In some embodiments, the disclosure provides an engineered cell comprising the non-natural OLS described herein, e.g., having at least 90% sequence identity to any of SEQ ID NOs:2-49 and comprising an amino acid substitution at an amino acid position corresponding to position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the engineered cell is a bacterial cell. In some embodiments, the engineered cell is not a yeast cell. Exemplary engineered cells are provided herein.

In some embodiments, the disclosure provides an engineered cell comprising an OLS of any of SEQ ID NOs:2-49, e.g., wherein the engineered cell is, e.g., a bacterial cell. In some embodiments, the OLS comprises any of SEQ ID NOs:2, 3, 4, 6, 7, 8, 9, 11, 13, 14, 15, or 20. In some embodiments, the OLS comprises any of SEQ ID NOs:4, 6, 8, 9, 11, 13, 15, or 20. In some embodiments, the OLS comprises any of SEQ ID NOs:2, 3, 6, or 8. In some embodiments, the OLS comprises any of SEQ ID NOs:6 or 8. In some embodiments, the OLS comprises SEQ ID NO:2. In some embodiments, the OLS comprises SEQ ID NO:6. In some embodiments, the OLS comprises an amino acid variation as described herein, e.g., at an amino acid position corresponding to position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

In some embodiments, the disclosure provides an engineered cell comprising a non-naturally occurring OLS, wherein the OLS comprises at least 90%, at least 91%, at least 92%, at least 93%, at least 94%, at least 95%, at least 96%, at least 97%, at least 98%, at least 99%, or 100% sequence identity to any of SEQ ID NOs: 10-45, SEQ ID NO:48, or SEQ ID NO:49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the OLS comprises at least 90% sequence identity to SEQ ID NO:2 or 6.

In some embodiments, the disclosure provides an engineered cell comprising a non-naturally occurring OLS, wherein the OLS comprises at least 95%, at least 96%, at least 97%, at least 98%, at least 99%, or 100% sequence identity to SEQ ID NO:2 or any of SEQ ID NO:4-49 sequence identity to SEQ ID NO:2 or any of SEQ ID NO:4-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the OLS comprises at least 90% sequence identity to SEQ ID NO:2 or 6.

In some embodiments, the amino acid variation in the non-natural OLS of the engineered cell comprises an amino acid substitution. Amino acid substitutions are further described herein. In some embodiments, the amino acid substitution in the OLS of the engineered cell comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution in the OLS of the engineered cell comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution in the OLS of the engineered cell comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

In some embodiments, the disclosure provides an engineered cell comprising a non-naturally occurring OLS, wherein the OLS comprises at least 90%, at least 91%, at least 92%, at least 93%, at least 94%, at least 95%, at least 96%, at least 97%, at least 98%, at least 99%, or 100% sequence identity to any of SEQ ID NOs:2-49, and further comprising an amino acid variation at an amino acid position corresponding to amino acid position 133, 134, 192, 193, 194, 196, 198, 214, 216, 218, 259, 266, 267, 268, 338, 340, and/or 380 of SEQ ID NO:6. In some embodiments, the OLS comprises at least 90% sequence identity to SEQ ID NO:2 or 6. In some embodiments, the amino acid variation comprises an amino acid substitution, wherein the amino acid substitution comprises S133A, S133G, S133W, G134H, E192D, T193S, T194A, T194E, T194N, T194Q, T194S, V196C, F198L, L214M, A216G, G218A, V259Q, V259W, V259Y, A266P, T267I, T267V, T267W, T267Y, L268M, L268V, M338L, M338T, S340A, V380L, or a combination thereof, wherein the amino acid position corresponds to SEQ ID NO:6.

In some embodiments, the disclosure provides a non-naturally occurring OLS comprising at least 90%, at least 91%, at least 92%, at least 93%, at least 94%, at least 95%, at least 96%, at least 97%, at least 98%, at least 99%, or 100% sequence identity to any of SEQ ID NOs:2-49, and further comprising an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6. In some embodiments, the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof. In some embodiments, the OLS comprises at least 90% sequence identity to SEQ ID NO:2 or 6.

In some embodiments, the disclosure provides an engineered cell comprising the polynucleotide described herein, e.g., that comprises: (a) a nucleic acid sequence encoding an olivetol synthase (OLS) of any of SEQ ID NOs:2-49; and (b) a heterologous regulatory element operably linked to the nucleic acid sequence. In some embodiments, the disclosure provides an engineered cell comprising the polynucleotide described herein, e.g., that comprises: (a) a nucleic acid sequence encoding an olivetol synthase (OLS) of any of SEQ ID NOs:2-49; and (b) a heterologous regulatory element operably linked to the nucleic acid sequence, e.g., a bacterial regulatory element. In some embodiments, the engineered cell comprises a polynucleotide encoding the non-naturally occurring OLS provided herein, e.g., comprising at least 90% or at least 95% sequence identity to any of SEQ ID NOs:2-49 and further comprising an amino acid variation described herein. Polynucleotides encoding OLS are further described herein. In some embodiments, the engineered cell comprises an expression construct that comprises the polynucleotide described herein.

In some embodiments, the polynucleotide is integrated into a genome of the cell. Methods of integrating exogenous polynucleotides into the genome of host cells are described herein. In some embodiments, the polynucleotide is present on an expression construct. In some embodiments, the engineered cell comprises a plasmid, wherein the plasmid comprises the polynucleotide. Bacterial regulatory elements, plasmids, and expression constructs are described herein.

In some embodiments, the engineered cell is capable of producing 3,5,7-trioxododecanoyl-CoA from Hex-CoA. In some embodiments, the engineered cell is capable of producing 3,5,7-trioxododecanoyl-CoA, olivetol, olivetolic acid, a cannabinoid, an isomer, analog, or derivative thereof, or a combination thereof. In some embodiments, the cannabinoid comprises CBGA, THCA, CBDA, CBCA, and/or an isomer, analog, or derivative thereof. In some embodiments, the engineered cell is further capable of producing olivetol, PDAL, HTAL, an isomer, analog, or derivative thereof, or a combination thereof.

In some embodiments, the disclosure provides a composition comprising (i) an OLS of any of SEQ ID NOs:2-49 and (ii) one or more of: Hex-CoA, 3,5,7-trioxododecanoyl-CoA, olivetol, olivetolic acid, a cannabinoid, olivetol, PDAL, HTAL, and/or an isomer, analog, or derivative thereof. In some embodiments, the disclosure provides an engineered cell comprising the composition.

In some embodiments, the engineered cell described herein further comprises an enzyme in a cannabinoid biosynthesis pathway. As shown in the exemplary cannabinoid biosynthesis pathway of FIG. 1, hexanoyl-CoA is combined with malonyl-CoA by OLS to form a tetraketide (e.g., 3,5,7-trioxododecanoyl-CoA), which is subsequently converted to olivetolic acid by OAC. Prenyltransferase catalyzes the condensation of olivetolic acid and geranyldiphosphate, also known as geranyl pyrophosphate or GPP, to form CBGA. CBGA can then be converted to various cannabinoid products, e.g., THCA by 49-tetrahydrocannabinolic acid synthase (THCAS), CBDA by cannabidiolic acid synthase (CBDAS), and CBCA by cannabichromenic acid synthase (CBCAS).

### Olivetolic Acid Cyclase

In some embodiments, the engineered cell of the present disclosure further comprises olivetolic acid cyclase (OAC). As described herein, OAC catalyzes the conversion of a tetraketide, e.g., 3,5,7-trioxododecanoyl-CoA or an analog thereof, to olivetolic acid or an analog thereof.

In some embodiments, the engineered cell expresses an exogenous or overexpresses an endogenous or exogenous OAC. In some embodiments, the OAC is a natural OAC, e.g., a wild-type OAC. In some embodiments, the OAC is a non-natural OAC. In some embodiments, the OAC comprises one or more amino acid substitutions relative to a wild-type OAC. In some embodiments, the one or more amino acid substitutions in the non-natural OAC increases the activity of the OAC as compared to a wild-type OAC. OAC and non-natural variants thereof are further discussed in, e.g., WO2020/247741.

In some embodiments, the OAC has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:50. Although the amino acid positions of OAC described herein are with reference to the corresponding amino acid sequence of SEQ ID NO:50, it is understood that the amino acid sequence of a non-natural OAC can include an amino acid variation at an equivalent position corresponding to a variant of SEQ ID NO:50. Methods of sequence alignment and identifying corresponding amino acid positions in a variant sequence are known in the field.

In some embodiments, the OAC comprises a variation at amino acid position H5,17, L9, F23, F24, Y27, V46, T47, Q48, K49, N50, K51, V59, V61, V66, E67, 169, Q70, 173, 174, V79, G80, F81, G82, D83, R86, W89, L92, 194, D96, or a combination thereof, wherein the amino acid position is relative to SEQ ID NO:50. In some embodiments, the variation is an amino acid substitution. In some embodiments, the variation is in a first peptide (e.g., a first monomer) of an OAC dimer. In some embodiments, the variation is in a second peptide (e.g., a second monomer) of an OAC dimer.

In some embodiments, the OAC is a dimer, wherein a first peptide of the dimer (e.g., a first monomer) comprises a variation at amino acid position H5,17, L9, F23, F24, Y27, V59, V61, V66, E67, 169, Q70, 173, 174, V79, G80, F81, G82, D83, R86, W89, L92, 194, D96, V46, T47, Q48, K49, N50, K51, or combination thereof, and wherein a second peptide (e.g., a second monomer) of the dimer comprises a variation at amino acid position V46, T47, Q48, K49, N50, K51, or combination thereof, wherein the position corresponds to SEQ ID NO:50. In some embodiments, the OAC forms a dimer, wherein a first peptide of the dimer comprises a variation at amino acid position L9, F23, V59, V61, V66, E67, 169, Q70, 173, 174, V79, G80, F81, G82, D83, R86, W89, L92, 194, V46, T47, Q48, K49, N50, K51, or combination thereof, and a second peptide of the dimer comprises a variation at amino acid position V46, T47, Q48, K49, N50, K51, or combination thereof, wherein the position corresponds to SEQ ID NO:50.

In some embodiments, the OAC comprises an amino acid substitution selected from H5X¹, wherein X¹ is G, A, C, P, V, L, I, M, F, Y, W, Q, E, K, R, S, T, Y, N, Q, D, E, K, or R; 17X², wherein X² is G, A, C, P, V, L, M, F, Y, W, K, R, S, T, H, N, Q, D, or E; L9X³, wherein X³ is G, A, C, P, V, I, M, F, Y, W, K, R, S, T, Y, H, N, Q, D, E, K, or R; F23X⁴, wherein X⁴ is G, A, C, P, V, L, I, M, Y, W, S, T, H, N, Q, D, E, K, or R; F24X⁵, wherein X⁵ is G, A, C, P, V, I, M, Y, S, T, H, N, Q, D, E, K, R, or W; Y27X⁶, wherein X⁶ is G, A, C, P, V, L, I, M, F, W, S, T, H, N, Q, D, E, K, or R; V59X⁷, wherein X⁷ is G, A, C, P, L, I, M, F, Y, W, H, Q, E, K, or R; V61X⁸, wherein X⁸ is G, A, C, P, L, I, M, F, Y, W, H, Q, E, K, R, S, T, N, or D; V66X⁹, wherein X′ is G, A, C, P, L, I, M, F, Y, or W; E67X¹⁰, wherein X¹⁰ is G, A, C, P, V, L, I, M, F, Y, or W; 169X¹¹, wherein X¹¹ is G, A, C, P, V, L, M, F, Y, or W; Q70X¹², wherein X¹² is S, T, H, N, D, E, R, K, or Y; 173X¹³, wherein X¹³ is G, A, C, P, V, L, M, F, Y, or W; 174X¹⁴, wherein X¹⁴ is G, A, C, P, V, L, M, F, Y, or W; V79X¹⁵, wherein X¹⁵ is G, A, C, P, L, I, M, F, Y, or W; G80X¹⁶, wherein X¹⁶ is A, C, P, V, L, I, M, F, Y, W, S, T, H, N, Q, D, E, K, or R; F81X¹⁷, wherein X¹⁷ is G, A, C, P, V, L, I, M, Y, W, S, T, H, N, Q, D, E, R, or K; G82X¹⁸, wherein X¹⁸ is A, C, P, V, L, I, M, F, Y, W, S, T, H, N, Q, E, K, or R; D83X¹⁹, wherein X¹⁹ is S, T, H, Q, N, E, R, K, or Y; R86X²⁰, wherein X²⁰ is S, T, H, Q, N, D, E, K, or Y; W89X²¹, wherein X²¹ is G, A, C, P, V, L, I, M, F, Y, W, S, T, H, N, Q, D, E, K, or R; L92X²², wherein X²² is G, A, C, P, V, I, M, F, Y, or W; 194X²³, wherein X²³ is G, A, C, P, V, L, M, F, Y, W, K, R, S, T, Y, H, N, Q, D, or E; D96X²⁴, wherein X²⁴ is S, T, H, Q, N, E, R, K, or Y; V46X²⁵, wherein X²⁵ is G, A, C, P, L, I, M, F, Y, or W; T47X²⁶, wherein X²⁶ is S, H, Q, N, D, E, R, K, or Y; Q48X²⁷, wherein X²⁷ is S, T, H, N, D, E, R, K, or Y; K49X²⁸, wherein X²⁸ is S, T, H, Q, N, D, E, R, or Y; N50X²⁹, wherein X²⁹ is G, A, C, P, V, L, I, M, F, Y, or W; K51X³⁰, wherein X³⁰ is S, T, H, Q, N, D, E, R, or Y; V46*X³¹, wherein X³¹ is G, A, C, P, L, I, M, F, Y, or W; T47*X³², wherein X³² is S, H, Q, N, D, E, R, K, or Y; Q48*X³³, wherein X³³ is S, T, H, N, D, E, R, K, or Y; K49*X³⁴, wherein X³⁴ is S, T, H, Q, N, D, E, R, or Y; N50*X³⁵, wherein X³⁵ is G, A, C, P, V, L, I, M, F, Y, or W; K51*X³⁶, wherein X³⁶ is S, T, H, Q, N, D, E, R, or Y; and a combination thereof; wherein the amino acid position corresponds to SEQ ID NO:8, and wherein the “*” following the amino acid position indicates amino acid residues from a second peptide of a OAC dimer (e.g., a second monomer) and corresponding to SEQ ID NO:50.

In some embodiments, the OAC described herein is capable of producing olivetolic acid at a faster rate compared with a wild-type OAC. In some embodiments, the OAC has increased affinity for a polyketide (e.g., 3,5,7-trioxododecanoyl-CoA or an analog thereof, as produced by an OLS described herein) compared with a wild-type OAC. In some embodiments, the rate of formation of olivetolic acid from 3,5,7-trioxododecanoyl-CoA or analog thereof by the OAC described herein is about 1.2 times to about 300 times, about 1.5 times to about 200 times, or about 2 times to about 30 times as compared to a wild-type OAC. The rate of formation of olivetolic acid from 3,5,7-trioxododecanoyl-CoA or an analog thereof can be determined in an in vitro enzymatic reaction using a purified OAC. Methods of determining enzyme kinetics and product formation rate are known in the field.

In some embodiments, the OAC is present in molar excess of the OLS in the engineered cell. In some embodiments, the molar ratio of the OLS to the OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or 1 to more than 10,000. In some embodiments, the molar ratio of the OLS to the OAC is about 1000:1, 500:1, 100:1, 10:1, 5:1, 2.5:1. 1.5:1, 1.2:1. 1.1:1, 1:1, or less than 1 to 1. In some embodiments, the enzyme turnover rate of the OAC is greater than OLS. As used herein, “turnover rate” refers to the rate at which an enzyme can catalyze a reaction (e.g., turn substrate into product). In some embodiments, the higher turnover rate of OAC compared to OLS provides a greater rate of formation of olivetolic acid than olivetol or other byproducts such as PDAL, HTAL, and other lactone analogs.

In some embodiments, the total byproducts (e.g., olivetol and analogs thereof, PDAL, HTAL, and other lactone analogs) of the OLS reaction products in the presence of molar excess of OAC, are in an amount (w/w) of less than about 50%, 45%, 40%, 35%, 30%, 25%, 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by the combination of individual OLS and OAC enzyme reactions.

In some embodiments, the disclosure provides a composition comprising the OLS described herein and the OAC described herein. In some embodiments, the disclosure provides an engineered cell comprising the OLS described herein and the OAC described herein. In some embodiments, the disclosure provides one or more polynucleotides comprising one or more nucleic acid sequences encoding the OLS described herein and the OAC described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the invention provides an engineered cell comprising the one or more polynucleotides. In some embodiments, the disclosure provides an engineered cell comprising the expression construct. In some embodiments, the engineered cell is capable of producing 3,5,7-trioxododecanoyl-CoA, olivetol, olivetolic acid, a cannabinoid, an isomer, analog, or derivative thereof, or a combination thereof. In some embodiments, the cannabinoid comprises CBGA, THCA, CBDA, CBCA, and/or an isomer, analog, or derivative thereof. In some embodiments, the engineered cell is further capable of producing olivetol, PDAL, HTAL, an analog, or derivative thereof, or a combination thereof.

### Prenyltransferase

In some embodiments, the engineered cell of the present disclosure further comprises a prenyltransferase. As described herein, prenyltransferase performs the conversion of olivetolic acid and GPP to CBGA (or an analogous reaction thereof, e.g., to produce CBGOA, CBGVA, CBGO, CBGV, or CBG). In (′. sativa, prenyltransferase is a transmembrane protein belonging to the UbiA superfamily of membrane proteins. Other prenyltransferases, e.g., aromatic prenyltransferases such as NphB from Streptomyces, which are non-transmembrane and soluble, can also catalyze conversion of olivetolic acid to CBGA.

In some embodiments, the engineered cell expresses an exogenous or overexpresses an endogenous or exogenous prenyltransferase. In some embodiments, the prenyltransferase is a natural prenyltransferase, e.g., wild-type prenyltransferase. In some embodiments, the prenyltransferase is a non-natural prenyltransferase. In some embodiments, the prenyltransferase comprises one or more amino acid substitutions relative to a wild-type prenyltransferase. In some embodiments, the one or more amino acid substitutions in the non-natural prenyltransferase increases the activity of the prenyltransferase as compared to a wild-type prenyltransferase. Prenyltransferase and non-natural variants thereof are further discussed in, e.g., WO2019/173770 and WO2021/046367.

In some embodiments, the prenyltransferase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:51. In some embodiments, the prenyltransferase is a non-natural prenyltransferase comprising at least 1, at least 2, at least 3, at least 4, at least 5, at least 6, at least 7, at least 8, at least 9, or at least 10 amino acid variations at positions corresponding to SEQ ID NO:51.

Although the amino acid positions of prenyltransferase described herein are with reference to the corresponding amino acid sequence of SEQ ID NO:51, it is understood that the amino acid sequence of a non-natural prenyltransferase can include an amino acid variation at an equivalent position corresponding to a variant of SEQ ID NO:51. Methods of sequence alignment and identifying corresponding amino acid positions in a variant sequence are known in the field.

In some embodiments, the prenyltransferase comprises an amino acid substitutions at position V45, V47, S49, F121, T124, Q159, M160, Y173, S212, V213, A230, 1232, T267, V269, Y286, T290, Q293, R294, L296, F300, or a combination thereof, wherein the position corresponds to SEQ ID NO:51. In some embodiments, the prenyltransferase comprises two or more amino acid substitutions at positions V45, V47, S49, F121, T124, Q159, M160, Y173, S212, V213, A230, 1232, T267, V269, Y286, T290, Q293, R294, L296, F300, or a combination thereof, wherein the position corresponds to SEQ ID NO:51.

In some embodiments, the amino acid substitution comprises S49T, F121L, T124R, Q159H, Q159R, Q159S, Q159T, Q159Y, Q159A, Q159F, Q159G, Q159I, Q159K, Q159L, Q159M, Q159A, S175H, S175K, S175R, S212H, 1232H, T267W, L268Y, A285Y, Y286A, Y286F, Y286L, Y286M, Y286P, Y286I, Y286T, Y286V, Q293F, Q293W, Q293H, Q293C, Q293A, Q293S, Q293V, Q293D, Q293Y, Q293E, Q2931, Q293M, Q293T, F300K, or a combination thereof, wherein the amino acid position corresponds to SEQ ID NO:51. In some embodiments, the amino acid substitution comprises V45I, V45T, F121V, T124K, T124L, Q159S, M160L, M160S, Y173D, Y173K, Y173P, Y173Q, S212H, A230S, T267P, Y286V, Q293H, R294K, L296K, L296L, L296M, L296Q, F300Y, or a combination thereof, wherein the amino acid position corresponds to SEQ ID NO:51.

In some embodiments, the prenyltransferase described herein is capable of a greater rate of formation of CBGA from GPP and olivetolic acid (or an analogous reaction thereof) as compared with wild-type prenyltransferase.

In some embodiments, the disclosure provides a composition comprising the OLS described herein and one or both of the OAC described herein and the prenyltransferase described herein. In some embodiments, the disclosure provides an engineered cell comprising the OLS described herein and one or both of the OAC described herein and the prenyltransferase described herein. In some embodiments, the disclosure provides one or more polynucleotides comprising the OLS described herein and one or both of the OAC described herein and the prenyltransferase described herein. In some embodiments, the disclosure provides an expression construct comprising the one or more polynucleotides. In some embodiments, the expression construct comprises more than one expression vector. In some embodiments, the invention provides an engineered cell comprising the one or more polynucleotides. In some embodiments, the disclosure provides an engineered cell comprising the expression construct. In some embodiments, the engineered cell is capable of producing 3,5,7-trioxododecanoyl-CoA, olivetol, olivetolic acid, a cannabinoid, an isomer, analog, or derivative thereof, or a combination thereof. In some embodiments, the cannabinoid comprises CBGA, THCA, CBDA, CBCA, and/or an isomer, analog, or derivative thereof. In some embodiments, the engineered cell is further capable of producing olivetol, PDAL, HTAL, an isomer, analog, or derivative thereof, or a combination thereof.

### Cannabinoid Synthase

In some embodiments, the engineered cell of the disclosure further comprises a cannabinoid synthase. As described herein, a cannabinoid synthase catalyzes the conversion of CBGA to THCA, CBDA, and/or CBCA (or an analogous reaction thereof, e.g., conversion of CBGOA to THCOA, CBDOA, and/or CBCOA; conversion of CBGVA to THCVA, CBDVA, and/or CBCVA; conversion of CBGO to THCO, CBDO, and/or CBCO; conversion of CBGV to THCV, CBDV, and/or CBCV; and/or conversion of CBG to THC, CBD, and/or CBC).

In some embodiments, the engineered cell expresses an exogenous or overexpresses an endogenous or exogenous cannabinoid synthase. In some embodiments, the cannabinoid synthase is a natural cannabinoid synthase, e.g., wild-type cannabinoid synthase. In some embodiments, the cannabinoid synthase is a non-natural cannabinoid synthase. In some embodiments, the cannabinoid synthase comprises tetrahydrocannabinolic acid synthase (THCAS), cannabidiolic acid synthase (CBDAS), cannabichromenic acid synthase (CBCAS), or combination thereof. Cannabinoid synthases and non-natural variants thereof are further discussed in, e.g., PCT/US2021/027125.

In some embodiments, the cannabinoid synthase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:52. In some embodiments, the cannabinoid synthase is a non-natural cannabinoid synthase comprising at least 1, at least 2, at least 3, at least 4, at least 5, at least 6, at least 7, at least 8, at least 9, or at least 10 amino acid variations at positions corresponding to SEQ ID NO:52.

In some embodiments, the cannabinoid synthase comprises an amino acid substitution at position K36, C37, K40, V46, Q58, L59, N89, N90, C99, K101, K102, K296, V321, V358, K366, K513, N516, N528, H544, or a combination thereof, wherein the position corresponds to SEQ ID NO:52. In some embodiments, the cannabinoid synthase comprises an amino acid substitution at one or both of C37 and C99, wherein the position corresponds to SEQ ID NO:52.

In some embodiments, the amino acid substitution comprises K36D, K36R, C37A, C37D, C37H, C37Y, C37E, C37K, C37N, C37Q, C37T, C37R, K40D, K40E, K40R, V46E, Q58E, L59T, N89D, N90D, N90T, C99F, C99A, C99I, C99V, C99L, K101D, K101E, K101R, K102D, K102E, K102R, K296E, V321T, V358T, K366D, K513D, N516E, N528T, H544Y, or a combination thereof, wherein the amino acid position corresponds to SEQ ID NO:52. In some embodiments, the amino acid substitution comprises a substitution selected from C37A, C37Q, C37N, C37E, C37D, C37R, and C37K; and a substitution selected from C99V, C99A, C99I and C99L. In some embodiments, the cannabinoid synthase described herein does not comprise a disulfide bond in its structure.

In some embodiments, the cannabinoid synthase is capable of converting CBGA to THCA, or an analogous reaction thereof. In some embodiments, the cannabinoid synthase is capable of converting CBGA to CBDA, or an analogous reaction thereof. In some embodiments, the cannabinoid synthase is capable of converting CBGA to CBCA, or an analogous reaction thereof.

In some embodiments, the disclosure provides a composition comprising the OLS described herein and one or more of the OAC described herein, the prenyltransferase described herein, and the cannabinoid synthase described herein. In some embodiments, the disclosure provides an engineered cell comprising the OLS described herein and one or more of the OAC described herein, the prenyltransferase described herein, and the cannabinoid synthase described herein. In some embodiments, the disclosure provides one or more polynucleotides comprising the OLS described herein and one or more of the OAC described herein, the prenyltransferase described herein, and the cannabinoid synthase described herein. In some embodiments, the disclosure provides an expression construct comprising the one or more polynucleotides. In some embodiments, the expression construct comprises more than one expression vector. In some embodiments, the invention provides an engineered cell comprising the one or more polynucleotides. In some embodiments, the disclosure provides an engineered cell comprising the expression construct. In some embodiments, the engineered cell is capable of producing 3,5,7-trioxododecanoyl-CoA, olivetol, olivetolic acid, a cannabinoid, an isomer, analog, or derivative thereof, or a combination thereof. In some embodiments, the cannabinoid comprises CBGA, THCA, CBDA, CBCA, and/or an isomer, analog, or derivative thereof. In some embodiments, the engineered cell is further capable of producing olivetol, PDAL, HTAL, an isomer, analog, or derivative thereof, or a combination thereof.

### Geranyl Pyrophosphate Biosynthesis

In some embodiments, the engineered cell of the disclosure further comprises an enzyme in a geranyl pyrophosphate (GPP) biosynthesis pathway.

GPP biosynthesis pathways are further described, e.g., in WO2017/161041. GPP biosynthesis pathways include, but are not limited to, a mevalonate (MVA) pathway, a non-mevalonate methylerythritol-4-phosphate (MEP) pathway, and an alternative non-MEP, non-MVA GPP pathway. In some embodiments, the engineered cell expresses an exogenous or overexpresses an endogenous or exogenous GPP biosynthesis pathway enzyme, thereby increasing production of GPP. In some embodiments, the increased production of GPP results in increased production of the cannabinoids described herein, e.g., CBGA, THCA, CBDA, CBCA, or an isomer, analog, or derivative thereof.

In some embodiments, the engineered cell produces GPP from a MVA pathway. In some embodiments, the engineered cell produces GPP from an alternative non-MEP, non-MVA GPP pathway. In some embodiments, the MVA pathway comprises an enzyme selected from acetoacetyl-CoA thiolase (AACT); HMG-COA synthase (HMGS); HMG-COA reductase (HMGR); mevalonate-3-kinase (MVK); phosphomevalonate kinase (PMK); mevalonate-5-pyrophosphate decarboxylase (MVD); isopentenyl pyrophosphate isomerase (IDI), and geranyl pyrophosphate synthase (GPPS).

In some embodiments, the engineered cell produces GPP from a MEP pathway. In some embodiments, the MEP pathway comprises an enzyme selected from 1-deoxy-D-xylulose 5-phosphate synthase (DXS), 1-deoxy-D-xylulose 5-phosphate reductoisomerase (DXR); 2-C-methyl-D-erythritol 4-phosphate cytidylyltransferase (CMS); 4-diphosphocytidyl-2-C-methyl-D-erythritol kinase (CMK); 2-C-methyl-D-erythritol 2,4-cyclodiphosphate synthase (MECS); 4-hydroxy-3-methyl-but-2-enyl pyrophosphate synthase (HDS); 4-hydroxy-3-methyl-but-2-enyl pyrophosphate reductase (HDR); isopentenyl pyrophosphate isomerase (IDI), and geranyl pyrophosphate synthase (GPPS).

In some embodiments, the engineered cell produces GPP from an alternative non-MEP, non-MVA GPP pathway. In some embodiments, GPP is produced from a precursor selected from isoprenol, prenol, and geraniol. In some embodiments, the non-MVA, non-MEP pathway comprises an enzyme selected from alcohol kinase, alcohol diphosphokinase, phosphate kinase, isopentenyl diphosphate isomerase, and geranyl pyrophosphate synthase (GPPS).

In some embodiments, the GPP biosynthesis pathway enzyme comprises geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, or a combination thereof.

In some embodiments, the disclosure provides a composition comprising the OLS described herein and one or more of the OAC described herein, the prenyltransferase described herein, the cannabinoid synthase described herein, and the GPP biosynthesis pathway enzyme described herein. In some embodiments, the disclosure provides an engineered cell comprising the OLS described herein and one or more of the OAC described herein, the prenyltransferase described herein, the cannabinoid synthase described herein, and the GPP biosynthesis pathway enzyme described herein. In some embodiments, the disclosure provides one or more polynucleotides comprising the OLS described herein and one or more of the OAC described herein, the prenyltransferase described herein, the cannabinoid synthase described herein, and the GPP biosynthesis pathway enzyme described herein. In some embodiments, the disclosure provides an expression construct comprising the one or more polynucleotides. In some embodiments, the expression construct comprises more than one expression vector. In some embodiments, the invention provides an engineered cell comprising the one or more polynucleotides. In some embodiments, the disclosure provides an engineered cell comprising the expression construct. In some embodiments, the engineered cell is capable of producing 3,5,7-trioxododecanoyl-CoA, olivetol, olivetolic acid, a cannabinoid, an isomer, analog, or derivative thereof, or a combination thereof. In some embodiments, the cannabinoid comprises CBGA, THCA, CBDA, CBCA, and/or an isomer, analog, or derivative thereof. In some embodiments, the engineered cell is further capable of producing olivetol, PDAL, HTAL, an isomer, analog, or derivative thereof, or a combination thereof.

### Additional Strain Modifications

In some embodiments, the engineered cell of the disclosure further comprises a modification that facilitates the production of the cannabinoids described herein, e.g., CBGA, THCA, CBDA, CBCA, and/or an isomer, analog, or derivative thereof. In some embodiments, the modification increases production of a cannabinoid in the engineered cell compared with a cell not comprising the modification. In some embodiments, the modification increases efflux of a cannabinoid in the engineered cell compared with a cell not comprising the modification. In some embodiments, the cannabinoid is CBGA, THCA, CBDA, CBCA, and/or an isomer, analog, or derivative thereof. In some embodiments, the modification comprises expressing or upregulating the expression of an endogenous gene that facilitates production of a cannabinoid. In some embodiments, the modification comprises introducing and/or overexpression an exogenous and/or heterologous gene that facilitates production of a cannabinoid. In some embodiments, the modification comprises downregulating, disrupting, or deleting an endogenous gene that hinders production of a cannabinoid. Expression and/or overexpression of endogenous and exogenous genes, and downregulation, disruption and/or deletion of endogenous genes are described herein.

In some embodiments, the engineered cell of the invention comprises one or more of the following modifications:
i) express one or more exogenous nucleic acid sequences or overexpress one or more endogenous genes encoding a protein having an ABC transporter permease activity;
ii) express one or more exogenous nucleic acid sequences or overexpress one or more endogenous genes encoding a protein having an ABC transporter ATP-binding protein activity;
iii) express one or more exogenous nucleic acids sequences or overexpress one or more endogenous genes selected from blc, ydhC, ydhG, or a homolog thereof;
iv) express one or more exogenous nucleic acids sequences or overexpress one or more endogenous genes selected from mlaD, mlaE, mlaF, or a homolog thereof;
v) express one or more exogenous nucleic acid sequences or overexpress one or more endogenous genes encoding a protein having a siderophore receptor protein activity or overexpress one or more endogenous genes encoding a protein having a siderophore receptor protein activity;
vi) comprise a disruption of or downregulation in the expression of a regulator of expression of one or more endogenous genes encoding a protein having an ABC transporter permease activity, a protein having an ABC transporter ATP-binding protein activity, a blc gene, a ybhG protein, a ydhC protein, a mlaD protein, mlaE protein, mlaF protein, or a protein having a siderophore receptor protein activity;
vii) express one or more exogenous nucleic acids sequences or overexpress one or more endogenous genes encoding a multi-domain protein having acetyl-CoA carboxylase activity (MD-ACC);
viii) express one or more exogenous nucleic acids sequences or overexpress one or more endogenous genes encoding acetyl-CoA carboxyltransferase subunit a, biotin carboxyl carrier protein, biotin carboxylase, or acetyl-CoA carboxyltransferase subunit ß, or express one or more exogenous nucleic acids or overexpress one or more endogenous genes encoding acetyl-CoA carboxyltransferase, biotin carboxyl carrier protein, or biotin carboxylase activities;
ix) disruption of or downregulation in the expression of an endogenous gene encoding a protein having (acyl-carrier-protein)S-malonyltransferase activity, an endogenous gene encoding a protein having 3-hydroxypalmitoyl-(acyl-carrier-protein) dehydratase activity, or both;
x) express an exogenous nucleic acid sequence or overexpress an endogenous gene encoding a protein having fatty acyl-CoA ligase activity, or both;
xi) disruption of or downregulation in the expression of at least one endogenous gene encoding a protein having acyl-CoA dehydrogenase activity or enoyl-CoA hydratase activity;
xii) comprise a disruption of or downregulation in the expression of at least one endogenous gene encoding a protein having acyl-CoA esterase/thioesterase activity;
xiii) comprise a disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway;
xiv) express an exogenous nucleic acid sequence or overexpress an endogenous gene encoding a protein having geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, prenol kinase activity, prenol diphosphokinase activity, isoprenol kinase activity, isoprenol diphosphokinase activity, dimethylallyl phosphate kinase activity, isopentenyl phosphate kinase activity, or isopentenyl diphosphate isomerase activity;
xv) express an exogenous nucleic acid sequence or overexpress an endogenous gene encoding a protein having GPP synthase activity;
xvi) express an exogenous nucleic acid sequence encoding an olivetol synthase;
xvii) express an exogenous nucleic acid sequence encoding an olivetolic acid cyclase;
xviii) express an exogenous nucleic acid sequence encoding a prenyltransferase;
xix) express one or more exogenous nucleic acid sequences or overexpressing one or more endogenous genes encoding one or more enzymes of MVA pathway, MEP pathway, or a non-MVA, non-MEP pathway;
xx) express an exogenous nucleic acid sequence or overexpress an endogenous gene encoding a biotin-(acetyl-CoA carboxylase) ligase;
xxi) express an exogenous nucleic acid sequence or overexpress an endogenous gene encoding a isopentenyl-diphosphate delta-isomerase;
xxii) express an exogenous nucleic acid sequence or overexpress an endogenous gene encoding a hydroxyethylthiazole kinase or both;
xxiii) express an exogenous nucleic acid sequence or overexpress an endogenous gene encoding a Type III pantothenate kinase; and
xxiv) comprise a disruption of or downregulation in the expression of at least one endogenous gene encoding a phosphatase selected from the group consisting of ADP-sugar pyrophosphatase, dihydroneopterin triphosphate diphosphatase, pyrimidine deoxynucleotide diphosphatase, pyrimidine pyrophosphate phosphatase, and Nudix hydrolase.

In some embodiments, the disclosure provides a composition comprising the OLS described herein and one or more of the OAC described herein, the prenyltransferase described herein, the cannabinoid synthase described herein, the GPP biosynthesis pathway enzyme described herein, and an additional modification described herein. In some embodiments, the disclosure provides an engineered cell comprising the OLS described herein and one or more of the OAC described herein, the prenyltransferase described herein, the cannabinoid synthase described herein, the GPP biosynthesis pathway enzyme described herein, and an additional modification described herein. In some embodiments, the disclosure provides one or more polynucleotides comprising the OLS described herein and one or more of the OAC described herein, the prenyltransferase described herein, the cannabinoid synthase described herein, the GPP biosynthesis pathway enzyme described herein, and an additional modification described herein. In some embodiments, the disclosure provides an expression construct comprising the one or more polynucleotides. In some embodiments, the expression construct comprises more than one expression vector. In some embodiments, the invention provides an engineered cell comprising the one or more polynucleotides. In some embodiments, the disclosure provides an engineered cell comprising the expression construct. In some embodiments, the engineered cell is capable of producing 3,5,7-trioxododecanoyl-CoA, olivetol, olivetolic acid, a cannabinoid, an isomer, analog, or derivative thereof, or a combination thereof. In some embodiments, the cannabinoid comprises CBGA, THCA, CBDA, CBCA, and/or an isomer, analog, or derivative thereof. In some embodiments, the engineered cell is further capable of producing olivetol, PDAL, HTAL, an isomer, analog, or derivative thereof, or a combination thereof.

### Host Cells

A variety of microorganisms may be suitable as the engineered cell described herein. Such organisms include both prokaryotic and eukaryotic organisms including, but not limited to, bacteria, including archaea and eubacteria, and eukaryotes, including yeast, plant, and insect. Non-limiting examples of suitable microbial hosts for the bioproduction of a cannabinoid include, but are not limited to, any Gram negative microorganism, in particular a member of the family Enterobacteriaceae, such as E. coli, Oligotropha carboxidovorans, or a Pseudomononas sp.; any Gram positive microorganism, e.g., Bacillus subtilis, Lactobaccilus sp., or Lactococcus sp.; a yeast, e.g., Saccharomyces cerevisiae, Pichia pastoris, or Pichia stipitis. In some embodiments, the microbial host is a member of the genus Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, or Saccharomyces. In some embodiments, the microbial host is Oligotropha carboxidovorans, Escherichia coli, Alcaligenes eutrophus (also known as (′upriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, or Saccharomyces cerevisiae. In some embodiments, the microbial host is E. coli.

Further exemplary species suitable as the engineered host cell are reported in U.S. Pat. No. 9,657,316 and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Lachancea kluyveri, Trichomonas vaginalis, Trypanosoma brucei, Pseudomonas stutzeri, Bradyrhizobium japonicum, Mesorhizobium loti, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp, paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In some embodiments, the engineered cell is a bacterial cell or a fungal cell. In some embodiments, the engineered cell is a bacterial cell. In some embodiments, the engineered cell is a yeast cell. In some embodiments, the engineered cell is an algal cell. In some embodiments, the engineered cell is a cyanobacterial cell. In some embodiments, the bacteria cell is an Escherichia, Corynebacterium, Bacillus, Ralstonia, Zymomonas, or Staphylococcus cell. In some embodiments, the bacterial cell is an Escherichia coli cell.

In some embodiments, the engineered cell is an organism selected from Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, (′arboxydothermus hydrogenoformans Z-2901, Caulobacter sp. AP07, Chloroflexus aggregans DSM 9485, Chloroflexus aurantiacus J-10-fl. Citrobacter freundii, Citrobacter koseri ATCC BAA-895, Citrobacter youngae, Clostridium species such as Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium boltede ATCC BAA-613, Clostridium carboxidivorans P7, Clostridium cellulovorans 743B, Clostridium difficile, Clostridium hiranonis DSM 13275, Clostridium hylemonde DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp, polymorphum ATCC 10953, Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp., Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas quensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, and Zea mays.

Algae that can be engineered for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like.

Microalgae (single-celled algae) produce natural oils that can contain the synthesized cannabinoids. Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditiana, Dunaliella salina, Dunaliella tertiolecta, Chlorella vulgaris, Chlorella variabilis, and (′hlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Platymonas, Pleurochrsis, Pleurococcus, Prototheca, Pseudochlorella, Pyramimonas, Pvrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Sevtonema, Spirulina, Stanieria, Starria, Stigonema, Symploca, Synechococcus, Svnechocystis, Tolipothrix, Trichodesmium. Tychonema, and Xenococcus species.

The host cell may be genetically modified for a recombinant production system, e.g., to produce 3,5,7-trioxododecanoyl-CoA, olivetol, olivetolic acid, a cannabinoid, and/or an isomer, analog, or derivative thereof as described herein. In some embodiments, to genetically modify a host cell disclosed herein, a polynucleotide described herein is introduced stably or transiently into the host cell using established techniques. Such techniques may include, but are not limited to, electroporation, conjugation, transduction, natural transformation, calcium phosphate precipitation, DEAE-dextran mediated transfection, liposome-mediated transfection, particle bombardment, and the like. For stable transformation, the polynucleotide generally includes a selectable marker, e.g., any of several well-known selectable markers such as neomycin resistance, ampicillin resistance, tetracycline resistance, chloramphenicol resistance, kanamycin resistance, hygromycin resistance, G418 resistance, bleomycin resistance, zeocin resistance, and the like. A broad range of plasmids and drug resistance markers are available and described herein. The cloning vectors are tailored to the host organisms based on the nature of antibiotic resistance markers that can function in that host cell. In some embodiments, the host cell is genetically modified using CRISPR to produce the engineered cell of the invention.

### Cell Culture

In some embodiments, the disclosure provides a method of producing 3,5,7-trioxododecanoyl-CoA, olivetol, olivetolic acid, a cannabinoid, and/or an isomer, analog, or derivative thereof, comprising culturing an engineered cell provided herein. In some embodiments, the method further comprises recovering the 3,5,7-trioxododecanoyl-CoA, olivetol, olivetolic acid, a cannabinoid, and/or an isomer, analog, or derivative thereof from the cell or cell extract, cell culture medium, whole culture, or combination thereof. In some embodiments, the cannabinoid comprises CBGA, THCA, CBDA, CBCA, and/or an isomer, analog, or derivative thereof.

In some embodiments, the culture medium of the engineered cell further comprises a carbon source. In embodiments, the culture medium comprises a carbon source that is also a primary energy source, i.e., a feed molecule. In some embodiments, the culture medium comprises one, two, three, or more carbon sources that are not primary energy source. Non-limiting examples of feed molecules that can be included in the culture medium include acetate, malonate, oxaloacetate, aspartate, glutamate, beta-alanine, alpha-alanine, butanoic acid, butyrate, hexanoic acid, hexanoate, hexanol, prenol, isoprenol, and geraniol. Further examples of compounds that can be provided in the culture medium include, without limitation, biotin, thiamine, pantotheine, and 4-phosphopantetheine. In some embodiments, the culture medium comprises hexanoic acid.

In some embodiments, the culture medium comprises acetate. In some embodiments, the culture medium comprises butyrate. In some embodiments, the culture medium comprises hexanoate. In some embodiments, the culture medium comprises hexanoic acid. In some embodiments, the culture medium comprises acetate, hexanoate, and/or hexanoic acid. In some embodiments, the culture medium comprises malonate, hexanoate, and/or hexanoic acid. In some embodiments, the culture medium comprises prenol, isoprenol, and/or geraniol. In some embodiments, the culture medium comprises aspartate, hexanoate or hexanoic acid, and prenol, isoprenol, and/or geraniol.

Depending on the desired microorganism or strain to be used, the appropriate culture medium may be used. For example, descriptions of various culture media may be found in “Manual of Methods for General Bacteriology,” American Society for Bacteriology (Washington D.C., USA, 1981). As used herein, culture medium, or simply “medium” as it relates to the growth source, refers to the starting medium, which may be in a solid or liquid form. “Culture medium” as used herein refers to medium (e.g. liquid medium) containing microbes that have been fermentatively grown and can include other cellular biomasses. The medium generally includes one or more carbon sources, nitrogen sources, inorganic salts, vitamins and/or trace elements. “Whole culture” as used herein refers to cultured cells plus the culture medium in which they are cultured. “Cell extract” as used herein refers to a lysate of the cultured cells, which may include the culture medium and which may be crude (unpurified), purified or partially purified. Methods of purifying cell lysates are known to the skilled artisan and described in embodiments herein.

Exemplary carbon sources include sugar carbons such as sucrose, glucose, galactose, fructose, mannose, isomaltose, xylose, maltose, arabinose, cellobiose and 3-, 4-, or 5-oligomers thereof. Other carbon sources include carbon sources such as methanol, ethanol, glycerol, formate and fatty acids. Still other carbon sources include carbon sources from gas such as synthesis gas, waste gas, methane, CO, CO² and any mixture of CO, CO² with H₂. Other carbon sources can include renewal feedstocks and biomass. Exemplary renewal feedstocks include cellulosic biomass, hemicellulosic biomass, and lignin feedstocks.

In some embodiments, the engineered cell is sustained, cultured, or fermented under aerobic, microaerobic, anaerobic or substantially anaerobic conditions. Exemplary aerobic, microaerobic, and anaerobic conditions have been described previously and are known in the art. Exemplary anaerobic conditions for fermentation processes are described, for example, in U.S. Patent Publication No. 2009/0047719.

The culture conditions can be scaled up and grown continuously for manufacturing the cannabinoid products described herein. Exemplary growth procedures include, for example, fed-batch fermentation and batch separation; fed-batch fermentation and continuous separation, or continuous fermentation and continuous separation. Fermentation procedures can be particularly useful for the biosynthetic production of commercial quantities of cannabinoids. Examples of batch and continuous fermentation procedures are known in the field. Typically, cells are grown at a temperature in the range of about 25° C. to about 40° C. in an appropriate medium, or up to about 70° ° C. for thermophilic microorganisms. Generally, and as with non-continuous culture procedures, the continuous and/or near-continuous production of cannabinoid product can include culturing a cannabinoid-producing organism with sufficient nutrients and medium to sustain and/or nearly sustain growth in an exponential phase. Continuous culture under such conditions can include, for example, 1 day, 2, 3, 4, 5, 6 or 7 days or more. In some embodiments, the organism is cultured for 1 week, 2, 3, 4 or 5 or more weeks and up to several months. In some embodiments, the organism is cultured for 1 hour to 1 day. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose. In some embodiments, the cannabinoid is CBGA, THCA, CBDA, CBCA, an isomer, analog, or derivative thereof, or a combination thereof.

The culture medium at the start of fermentation may have a pH of about 4 to about 7. The pH may be less than 11, less than 10, less than 9, or less than 8. In some embodiments, the pH is at least 2, at least 3, at least 4, at least 5, at least 6, or at least 7. In some embodiments, the pH of the medium is about 6 to about 9.5; 6 to about 9, about 6 to 8 or about 8 to 9.

In some embodiments, upon completion of the cultivation period, the fermenter contents are passed through a cell separation unit, for example, a centrifuge or filtration unit, to remove cells and cell debris. In embodiments where the desired product is expressed intracellularly, the cells are lysed or disrupted enzymatically or chemically prior to or after separation of cells from the fermentation broth, as desired, in order to release additional product. The fermentation broth can be transferred to a product separations unit. Isolation of product can be performed by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, methylene chloride, chloroform, benzene, pentane, hexane, heptane, petroleum ether, methyl tertiary butyl ether (MTBE),, and the like) to provide an organic solution of the product, if appropriate, standard distillation methods, and the like, depending on the chemical characteristics of the product of the fermentation process.

Suitable purification and/or assays to test a cannabinoid produced by the methods herein, e.g., CBGA, THCA, CBDA, CBCA, and/or an isomer, analog, or derivative thereof, can be performed using known methods. For example, product and byproduct formation in the engineered production host can be monitored. The final product and intermediates, and other organic compounds, can be analyzed by methods such as HPLC, GC-MS, LC-MS, or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al. (2005), Biotechnol. Bioeng. 90:775-779), or other suitable assay and detection methods well known in the art. The individual enzyme or protein activities from the exogenous DNA sequences can also be assayed using methods known in the art.

The cannabinoids produced using methods described herein, e.g., CBGA, THCA, CBDA, CBCA, and/or an isomer, analog, or derivative thereof, can be separated from other components in the culture using a variety of methods well known in the art. Such separation methods include, for example, extraction procedures, e.g., liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. For example, the amount of cannabinoid or other products, e.g., 3,5,7-trioxododecanoyl-CoA, olivetol, olivetolic acid, or a byproduct such as olivetol, PDAL, HTAL, or an isomer, analog, or derivative thereof, produced in a bio-production media generally can be determined using any of methods such as, for example, high performance HPLC, GC, GC-MS, or spectrometry.

In some embodiments, the cell extract or cell culture medium described herein comprises 3,5,7-trioxododecanoyl-CoA, olivetol, olivetolic acid, a cannabinoid, and/or an isomer, analog, or derivative thereof. In some embodiments, the cell extract or cell culture medium described herein comprises a cannabinoid. In some embodiments, the cannabinoid is cannabichromene (CBC) type (e.g. cannabichromenic acid), cannabigerol (CBG) type (e.g. cannabigerolic acid), cannabidiol (CBD) type (e.g. cannabidiolic acid), Δ9-trans-tetrahydrocannabinol (Δ9-THC) type (e.g. Δ9-tetrahydrocannabinolic acid), Δ8-trans-tetrahydrocannabinol (Δ8-THC) type, cannabicyclol (CBL) type, cannabielsoin (CBE) type, cannabinol (CBN) type, cannabinodiol (CBND) type, cannabitriol (CBT) type, or a combination thereof. In some embodiments, the cannabinoid is cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), or a combination thereof. In some embodiments, the cannabinoid is cannabichromenic acid (CBCA), cannabichromene (CBC), cannabichromevarinic acid (CBCVA), cannabichromevarin (CBCV), or a combination thereof. In some embodiments, the cannabinoid is cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), or a combination thereof. In some embodiments, the cannabinoid is Δ9-tetrahydrocannabinolic acid A (THCA-A), Δ9-tetrahydrocannabinolic acid B (THCA-B), Δ9-tetrahydrocannabinol (THC), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), 47-cis-iso-tetrahydrocannabivarin, Δ8-tetrahydrocannabinolic acid (Δ8-THCA), Δ8-tetrahydrocannabinol (Δ8-THC), or a combination thereof. In some embodiments, the cannabinoid is cannabicyclolic acid (CBLA), cannabicyclol (CBL), cannabicyclovarin (CBLV), cannabielsoic acid A (CBEA-A), cannabielsoic acid B(CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN—C4), cannabivarin (CBV), cannabinol-C2 (CNB—C2), cannabiorcol (CBN—C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), Δ9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy- Δ9-tetrahydrocannabinol (triOH-THC), or a combination thereof.

In some embodiments, the disclosure provides a cell extract or cell culture medium comprising 3,5,7-trioxododecanoyl-CoA, olivetol, olivetolic acid, a cannabinoid, and/or an isomer, analog, or derivative thereof. In some embodiments, the cannabinoid is CBGA, THCA, CBDA, CBCA, an isomer, analog, or derivative thereof, or a combination thereof, wherein the cell extract or cell culture medium is derived from the engineered cell described herein. In some embodiments, cell extract or cell culture medium further comprises olivetol, PDAL, HTAL, an isomer, analog, or derivative thereof, or a combination thereof.

### Methods

In some embodiments, the disclosure provides a method of making 3,5,7-trioxododecanoyl-CoA, olivetol, olivetolic acid, a cannabinoid, and/or an isomer, analog, or derivative thereof, comprising culturing the engineered cell described herein. In some embodiments, the engineered cell is cultured in the presence of hexanoic acid or hexanoate. In some embodiments, the disclosure provides a method of making 3,5,7-trioxododecanoyl-CoA, olivetol, olivetolic acid, a cannabinoid, and/or an isomer, analog, or derivative thereof, comprising isolating the 3,5,7-trioxododecanoyl-CoA, olivetol, olivetolic acid, a cannabinoid, and/or an isomer, analog, or derivative thereof from the cell extract or cell culture medium described herein. In some embodiments, the cannabinoid is CBGA, THCA, CBDA, CBCA, an isomer, analog, or derivative thereof, or a combination thereof. In some embodiments, the method further comprises isolating the 3,5,7-trioxododecanoyl-CoA, olivetol, olivetolic acid, a cannabinoid, and/or an isomer, analog, or derivative thereof.

Methods of culturing cells, e.g., the engineered cell of the invention, are provided herein. Methods of isolating 3,5,7-trioxododecanoyl-CoA, olivetol, olivetolic acid, a cannabinoid, and/or an isomer, analog, or derivative thereof are also provided herein. In some embodiments, the isolating comprises liquid-liquid extraction, pervaporation, evaporation, filtration, membrane filtration (e.g., reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and/or recrystallization, centrifugation, extractive filtration, ion exchange chromatography, size exclusion chromatography, adsorption chromatography, carbon adsorption, hydrogenation, ultrafiltration, or a combination thereof.

In some embodiments, the disclosure provides a method of making 3,5,7-trioxododecanoyl-CoA or an isomer, analog, or derivative thereof, comprising contacting hexanoyl-CoA and malonyl-CoA with an OLS described herein. In some embodiments, the method makes 3,5,7-trioxododecanoyl-CoA, olivetol, PDAL, HTAL, an isomer, analog, or derivative thereof, or a derivative thereof.

### Compositions

In some embodiments, the disclosure provides a composition comprising 3,5,7-trioxododecanoyl-CoA, olivetol, olivetolic acid, a cannabinoid, and/or an analog or derivative thereof, wherein the 3,5,7-trioxododecanoyl-CoA, olivetol, olivetolic acid, a cannabinoid, and/or an analog or derivative thereof is produced from the engineered cell described herein; isolated from the cell extract or cell culture medium described herein; or made by the method described herein.

In some embodiments, the composition comprises 3,5,7-trioxododecanoyl-CoA and olivetolic acid. In some embodiments, the composition comprises 3,5,7-trioxododecanoyl-CoA, olivetol, and olivetolic acid. In some embodiments, the composition comprises 3,5,7-trioxododecanoyl-CoA, olivetolic acid, and a byproduct of an OLS and/or OAC reaction such as olivetol, PDAL, HTAL, or an isomer, analog, or derivative thereof. In some embodiments, the composition comprises 3,5,7-trioxododecanoyl-CoA, olivetolic acid, a cannabinoid, and a byproduct of an OLS and/or OAC reaction.

In some embodiments, the disclosure provides a cannabinoid produced by the engineered cell described herein. In some embodiments, the disclosure provides a cannabinoid isolated from the cell extract or cell culture medium described herein. In some embodiments, the disclosure provides a cannabinoid made by the method described herein.

Exemplary cannabinoid compounds are described herein. In some embodiments, the composition comprises a cannabinoid selected from cannabigerolic acid (CBGA), tetrahydrocannabinolic acid (THCA), cannabidiolic acid (CBDA), cannabichromenic acid (CBCA), cannabigerol (CBG), tetrahydrocannabinol (THC), cannabidiol (CBD), cannabichromene (CBC), an analog or derivative thereof, or a combination thereof. In some embodiments, the cannabinoid comprises CBCA, CBDA, THCA, CBCOA, CBDOA, THCOA, CBCVA, CBDVA, THCVA, CBC, CBD, THC, or an isomer, analog or derivative thereof, or a combination thereof.

In some embodiments, the cannabinoid is 10% or greater, 20% or greater, 30% or greater, 40% or greater, 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total compound(s) in the composition.

In some embodiments, the composition is a therapeutic or medicinal composition. In some embodiments, the composition further comprises a pharmaceutically acceptable excipient. In some embodiments, the composition is a topical composition. In some embodiments, the composition is in the form of a cream, a lotion, a paste, or an ointment.

In some embodiments, the composition is an edible composition. In some embodiments, the composition is provided in a food or beverage product. In some embodiments, the composition is an oral unit dosage composition. In some embodiments, the composition is provided in a tablet or a capsule.

In some embodiments, the disclosure provides a composition comprising (i) an OLS described herein (e.g., any of SEQ ID NOs:2-49, and, e.g., comprising an amino acid variation as described herein) and (ii) one or more of: Hex-CoA, 3,5,7-trioxododecanoyl-CoA, olivetol, olivetolic acid, a cannabinoid, olivetol, PDAL, HTAL, and/or an isomer, analog, or derivative thereof. In some embodiments, the OLS comprises at least 90% sequence identity to any one of SEQ ID NOs:2-49 and further comprises an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6. In some embodiments, the composition further comprises an OAC, a prenyltransferase, a cannabinoid synthase, a GPP biosynthesis pathway enzyme, an additional modification described herein, or combination thereof. OAC, prenyltransferase, cannabinoid synthase, and GPP biosynthesis pathway enzyme are further described herein.

All references cited herein, including patents, patent applications, papers, textbooks and the like, and the references cited therein, to the extent that they are not already, are hereby incorporated herein by reference in their entirety.

Sequences

SEQ ID NO: 001
B1Q2B6 OLIS_CANSA 3,5,7-trioxododecanoyl-CoA synthase Cannabis sativa
MNHLRAEGPASVLAIGTANPENILLQDEFPDYYFRVTKSEHMTQLKEKFRKICDKSMIRKRNCFLNEEHLKQNPRLVEHE
MQTLDARQDMLVVEVPKLGKDACAKAIKEWGQPKSKITHLIFTSASTTDMPGADYHCAKLLGLSPSVKRVMMYQLGCYGG
GTVLRIAKDIAENNKGARVLAVCCDIMACLFRGPSESDLELLVGQAIFGDGAAAVIVGAEPDESVGERPIFELVSTGQTI
LPNSEGTIGGHIREAGLIFDLHKDVPMLISNNIEKCLIEAFTPIGISDWNSIFWITHPGGKAILDKVEEKLHLKSDKFVD
SRHVLSEHGNMSSSTVLFVMDELRKRSLEEGKSTTGDGFEWGVLFGFGPGLTVERVVVRSVPIKY
SEQ ID NO: 002
AAZ32094.1 [Oncidium hybrid cultivar]
MPSLESVKNAKRAEGFASILAIGRANPEHFIEQSSYPDFFFRVTNSEHLVALKKKFQRICDKTAIRKRHFAWNEELLTTN
SCFQTFMGNSLNVRQEFAIREIPKLGAQAANKAIQEWGQPKSRITHLIFCTTSGMDLPGADYQLTEILGLNPNVERVMLY
QQGCFAGGTTLRLAKCLAESRKGARVLVVCAETTTVLFRAPSEEHQDDLVTQALFADGASALIVGADPDEAAHERASFII
VSTSQVLLPDSAGAIGGHVSEGGLLATLHRDVPQIVSKNVGKCLEEAFTPLGISDWNSIFWVPHPGGRAILDQIEERVGL
KPEKLFISRHVLAEYGNMSSVCVHFALDEMRKRSAKEGKATTGEGLDWGVLFGFGPGLTVETVVLHSVPVTN
SEQ ID NO: 003
AIM58716.1 [Cymbidium hybrid cultivar]
MPSLESVKKSNRADGFASILAIGRANPENFIEQSTYPDFFFRVTNSEHLVNLKKKFQRICDKTAIRKRHFVWNEELLNAN
PCLGTFMDNSLNVRQEFAIREIPKLGAEAATKAIQEWGQPKSRITHLIFCTTSGMDLPGADYQLTQILGLNPNIERVMLY
QQGCFAGGTTLRLAKCLAESRKGARVLVVCAETTAVLFRAPSEEHQDDLVTQALFADGASALIVGADPDETAHERASFVI
VSTSQVLLPDSAGAIGGHVSEGGLIATLHRDVPQIVSKNVGKCLEEAFTPLGISDWNSIFWVPHPGGRAILDQVEERVGL
KPEKLIVSRHVLAEYGNMSSVCVHFALDEMRKRSKKEGKATTGEGLDWGVLFGFGPGLTVETVVLHSVPI
SEQ ID NO: 004
XP_020704098.1 [Dendrobium catenatum]
MPSLESIKKAPRADGFASILAIGRANPENFIEQSAYPDLFFRITNSEHLVDLKNKFKRICDKTAIRKRHFVWTEEFITAN
PCFSTFMDKSLNIRQEVAIREIPKLGAEAATKAIQEWGQPKSRITHLIFCTTSGMDLPGADYQLTQILGLNPNVERVMLY
QQGCFAGGTTIRLAKCLAESRKGARVLVVCAETTTVLFRGPSEEHQDDLVTQALFADGASALIVGADPDEAADEHASFVI
VSTSQVLLPDSAGAIGGHVSEGGLLATLHRDVPQIVSKNVGKCLEEAFTPLGISDWNSIFWVPHPGGRAILDQVEESVGL
KPEKLFISRHVLAEYGNMSSVCVHFALDEMRKRSAKEGKATTGEGLEWGVLFGFGPGVTVETVILRSVPI
SEQ ID NO: 005
XP_020572025.1 [Phalaenopsis equestris]
MPSFPSVKKAPTAEGFASILAIGRANPENFIEQSAYPDFFFRVTNSEHLVDLKKKFQRICDKTAIRKRHFVWNEEFLTAN
PCFSTFMDKSLNVRQEVAIREIPKLGAKAATKAIEDWGQPKSRITHLIFCTTSGMDLPGADYQLTQILGLNPNVERVMLY
QQGCFAGGTTLRLAKCLAESRKGARVLVVCAETTTVLFRAPSEEHQDDLVTQALFADGASAVIAVIVGADPDEAADERAS
FVIVSASQVLLPDSAGAIGGHVSEGGLLATLHRDVPQIVSKNVGKCLEEAFTPFGISDWNSIFWVPHPGGRAILDQVEER
VGLKPEKLSVSRHVLAEYGNMSSVCVHFALDEMRKRSAKEGKATTGEGLEWGVLFGFGPGLTVETVVLQSVPI
SEQ ID NO: 006
QDX46968.1 [Anoectochilus roxburghii]
MPSLESIRKAPRADGLASILAIGRANPDNFMEQSSFPDFFFRITGSDHLVDLKKKFQRICDRTAIRKRHFVWNEEFIKAN
PCFSTFMDNSLNVRQEVAIREIPKLGAEAATKAIKEWGQPKSRITHLIFCTTSGMDLPGADYQLTRILGLNPNVERVMLY
QQGCFAGGTTLRLAKCLAESRKGARVLVVCAETTTVLFRAPSEEHQEDLVTQALFADGASAVIVGADPDEEAHEKASFVI
FSTSQVLLPDSEGAIGGHVSEGGLLATLHRDVPQLVSKNVGKCLEEAFTPLGISDWNSIFWVPHPGGRAILDQIEERVGL
KPEKLTTSRHVLAEYGNMSSVCVHFVLDEMRKKSSKEGKATTGEGLEWGVLFGFGPGLTVETVVLRSVPL
SEQ ID NO: 007
AAX54693.1 [Phalaenopsis hybrid cultivar]
MPTIESIKKAPRAHGFASILAIGKANPENFIEQCHYPDFYFRVTSSEHLVDLKEKFQRMCDRTAIRKRHFVWNEDLLTAN
PCLRTYMDKSLNIRQEVAIREIPKLGAEAATKAIQEWGQPKSSITHLIFCTTSGMDLPGADFQLTQILGLNPNVERVMLY
QQGCFAGGTTLRLAKCLAESREGARVLVVCAETTTVVFRAPSEEHQDDLVTQALFADGASAVIVGVDPNEAAHERASFII
VSASQVLLPDSAGAIGGHVSEGGLTATLHRDVPQIVSKNVGKCLEEAFTPFGISDWNSIFWVPHAGGRAILDQVEERVGL
KPEKLSVSRHVLAEYGNMSSVCVHFALDEMRKKSAKEAKATTGEGLEWGVLFGFGPGLTVETVVLHSVPI
SEQ ID NO: 008
QCO76957.1 [Dendrobium officinale]
MPSLESIRKAPRANGFASILAIGRANPENFIEQSTYPDFFFRITNSEHLVDLKKKFQRICDKTAIRKRHFVWNEEFITTN
PCLHTFMDKSLDVRQEVAIREIPKLGAKAAAKAIQEWGQPKSRITHLIFCTTSGMDLPGADYQLTQILGLNPNVERVMLY
QQGCFAGGTTLRLAKCLAESRKGARVLVVCAETTTVLFRGPSEEHQEDLVTQALFADGASALIVGADPDEAAHERASFVI
VSTSQVLLPDSAGAIGGHVSEGGLLATLHRDVPKIVSKNVEKCLEEAFTPFGITDWNSIFWVPHPGGRAILDLVEERVGL
KPEKLLVSRHVLAEYGNMSSVCVHFALDEMRKRSAIEGKATTGEGLEWGVVFGFGPGLTVETVVLRSVPL
SEQ ID NO: 009
AHH25569.1 [Bletilla striata]
MPSLDSIKKAPRADGIASILAIGRANPDNIIEQSAYPDFYFRVTNSEHLVDLKKKFQRICEKTAIRKRHFVWNEEFLTSN
PSFSTFMDKSLYVRQEVAIREIPKLGAKAATKAIEDWGQPKSRISHLIFCTTSGMDLPGADYQLTQILGLNPNVERLMLY
EQGCFAGGTTLRLAKCLAESRKGARVLVVCAETTTVLFRAPSEEHQDDLVTQALFADGASALIVGADPDEAADERASFVI
VSTSQVLLPDTAGAIGGHVSEGGLLATLHRDVPQIVTKNVGKCLEEAFTPFGISDWNSIFWVPHPGGRAILDQVEERVGL
KPEKLSVSRHVLAEYGNMSSVCVHFALDEMRKRSANEGKPTTGEGLEWGVLFGFGPGLTVETVVLRSVPL
SEQ ID NO: 010
AAZ32093.1 [Oncidium hybrid cultivar]
MPSLESIKNAPKTDGFASILAIGSANPENIIEQSTYPDFFFHLTNSEHLVDLKNKFQRICDKTAIRKRHFAWNEELLTAN
PCLRTFMDNSLNVRQEFVIREIPKLGAQAATKAIQEWGQSKSRITHLIFSTTSGMDLPGADYQLTQILGLNPNIERVMLY
HQGCFAGGTTLRLTKSLAESRKGARVLVVCAETTSALTFRAPSEEHQDDLVVQALFGDGASALIVGADPDEAADERASFI
IVSTSQVLLSDSAGAIGGHLSEGGLIVTLHRDVPQIVSKNVGNCLKEAFTPLGISDWNSIFWVPHPGGPAILDQIEERVG
LNPEKLIISRHMLAEYGNMLGVSVHFALDEMRKRSAKEGKATTGEGLDWGVLLGIGPGITVETVVLHSVRI
SEQ ID NO: 011
XP_020704102.1 [Dendrobium catenatum]
MPSLESIKKAPRADGFASILAIGRANPENFIEQSAYPDLFFRITKSEHLVDLKNKFKRICDKTAIRKRHFVWTEEFITAN
PCFSTFMEKSLNIRQEVAIREIPKLGAEAAAKAIQEWGQPKSRITHLIFCTRSGMGLPGPDYQLTQILGLNPNVERVMIY
QQGCFAGGTTLRLAKCLAESHKGARVLVVCAETSTVLFRAPSMEHQEDLVTQALFADGASALIVGADPDETADEHASFVI
VSTSQVLLPESAGAIGGHVSEGGFLPMIHRDVPQIVSKNIGKCLEEAFTPLGIMDWNSIFWVPHPGGRAILDQLDERVGL
KPEKLFISRHVLKEYGNMSSASVHFALDEMRKWSAKEGTTGEGLEWGVLFGFGPGVTVDTVVLRSVPI
SEQ ID NO: 012
CAA10514.1 [Bromheadia finlaysoniana]
MASQVSPPSINMAPKADGFASILAIGRANPKNFIEQSTFPDFFFRVTNTEHMVDLKKKFQRICDKTSIRKRHFIWNEELL
TANPSLCTFMGNSLNLRHEVAVREIPKLGAEAATKAIQEWGQPKSFITHLVFCTTSGMDLPGADYQLTQILGLNLDIERV
MLHQQGCFLGGTTLRLAKYLAESRKGARVLVVCAETTTEFFRAPSEEHQEDLVTQSLFGDGASALIVGADPHEGARERAS
FILVSSSQVLLANSAHAITGHVSEGGIKATLHRDVPQIISNNLGKCLEEAFTPLGISDWNSIFWVLHPGGRAILDQVEEK
MGLEPEKLLISRHVLLEYGNMSSVCVHFALDEMRKRSSNEGKATTGEGLEWGVLFGFGPGLTIETVVLRSVSIS
SEQ ID NO: 013
AFU07718.1 [Paphiopedilum micranthum]
MPGLENRKKVEAPIRAEGLATIMAIGRANPPNAMEQSTFPDFYFRVTNSEHLVGLKKKFQRICEKTAIRRRHFVWNEEIL
NANPCLRTHMEPSLNVRQKIAVAEIPKMGAEAASRAIEEWGQSKSRITHLIFCTTSGMDLPGADYQLTRILGLNPNVQRV
MLYQQGCFAGGTVLRLAKCLAESQKGARVLVVCSETTAVLVRAPSEEYHDDLVTQALFADGASALIVGADPDEEAKERPI
FTIVSTTQVILPDSDGAIGGHLGEGGLTATLHRDVPLIISKNVSKCLEEAFTPLGISDWNSIFWAPHPGGRAILDQVEER
ASLKPEKLWASRHVLTEYGNMSSVCVHFVLDEIRKRSAKEGKATTGEGFDWGVLFGFGPGLTVETVVLRSVPLN
SEQ ID NO: 014
AFU07719.1 [Paphiopedilum purpuratum]
MPGLENRKKVEAPKRAEGLATILAIGRANPPNDMEQSTFPDFYFRVTNSEHLVSLKKKFERICEKTAIRRRHFVWNEEIL
NANPCLRTHMEPSLNVRQKIAVAEIPKLGAEAASRAIEEWGQPKSHITHLIFCTTSGMDLPGADYKLTRILGLNPNVQRV
MLYQQGCFAGGTVLRLAKCFAESQKGARVLVVCSETTTVLVRAPSEDYQDDLVTQALFADGASALIVGADPDEEAKEQPI
FTIVSATQVILPDSDGAIGGHLGEGGLTATLHRDVPLIISKNVSKCLEEAFGPLGISDWNSIFWAPHPGGRAILDQVEER
VGLKPEKLWASRHVLAEYGNMSSVCVHFVLDEIRKRSTKEGKTTTGEGFDWGVLFGFGPGLTVETVILRSVPLN
SEQ ID NO: 015
PKA53998.1 [Apostasia shenzhenica]
MPGLQIISKASSRAADGLAAILAIGRANPPNSMDQSSYPEFYFRVMDSDHLVDLKKKFQRICERTAIRKRHFVWNEELLR
DNPCLRTFMDSSLNVRQKVAVAEIPKLGAAAAERAIEEWGQPRSGITHLIFCTTSGMDLPGADYQLTKILGLNADVQRVM
LYQQGCFAGGTVLRLAKVLAESRKGARVLVVCAETTTVLIRAPSVEHQDDLVTQALFADGASALIVGADPVEEVNERPLF
SIISASQVILPDSDGAIGGHLGEGGLTATLHRDVPLIISKNVSKCLEDAFSPLGISDWNSIFWAPHPGGRAILDQVEERV
GLKPEKMWASRHVLAEYGNMSSVCVHFVLDEMRKRSAKEGKPTTGEGLEWGVLFGFGPGLTVETVVLRSHPIN
SEQ ID NO: 016
AFU07709.1 [Paphiopedilum armeniacum]
MPGLENRKKVEAPKREEGLATIMAIGRANPPNAMEQSTFPDFYFRVTNSEHMVGLKKKFQRICEKTAIRRRHFVWNEEIL
NANPCMCTHMEPSLNVGQKIVVAEIPKLGAEAASRAIEVWGQPKSRITHLIFCTTSSMDLLGADYKITRILGLNLNVQRG
MLYQQGCFAGGTVLRLAKCFTESQKGTRVLVVCSENAIILVRASSEDYQDDLVTQALFADGASALIVGEDPDEEAKERPI
FTIISTTHVILPDSDGAIGGHLGEGGLMATLQRDVPFIISKNVNTCLEESFAPLVISDWNSVFWAPHSGGRAILDQVEER
AGLKPEKLWASRHVLAKYGNMSSVCVHFVLDEIRKRSTKEGKTTTGEGFDWGVLFGFGLGLTVETVILRSVPLN
SEQ ID NO: 017
PKA56805.1 [Apostasia shenzhenica]
MPGVEAVAQNISPARSDGLAAILAIGRANPPNIVEQSSFADLYFRLHNSEHLVDLKKKLQRICDRTAIRKRHFVWDEELL
MANPCLRTVTEPSLNARQKVAITEIPKLGAAAATNAIAEWGRPKSDITHLIFCTTSGMDLPGADYQLIRLLGLNDNIQRI
MLYQQGCFAGGTVLRLAKVLAESRRSARVLIVCAETTTVLVRSPSVENQDDLVTQALFADGASALIVGADPNAGEKPVFS
VFSTSQVLLPDSDGAIGGHVGENGLTATLHRDVPAVISKNVGKCLEEAFTPLGISDWNSIFWAAHPGGRAILDQVEERVG
LKPEKMWASRHVLAEYGNMSSVSVHFALDEIRRRSAKEGKATTGDGFEWGVLFGFGPGLTVETVVLRSAPISA
SEQ ID NO: 018
PKU71375.1 [Dendrobium catenatum]
MAQRADGSASILAIGKATPENFLEQSTYPDYFFRVTNSQHLIDLKKKFQRICDKTSIRKRHFILNEELITKNPCLSKFME
NSINTRLEIYAKEIQKLAVEAATKAIQEWGQPKSCITHLIFSTLSDPGLPCGDYHLLQTLGLSPNIERVVILQHGCFAGG
TMLRLAKCLAESHKGARVLVVSAETTTMLFRGPSEEHQEDLITQALFADGASALIVGVNPNETIGERASFVITSASQVIL
PNSSHAITGHLSEGGIKATIHKDVPNLLSNNIGKILEEAFTPLGISNWNSIFWVVHPGGRAILDQLEERVGLKPEKLMIS
RHVLAEYGNLMGVCVHFVLDEMRKRSIDEGNTTTGEGLEWGVLLGFGLGVTIETIVLQSVSL
SEQ ID NO: 019
AFU07710.1 [Paphiopedilum x areeanum]
MPGLENRKKVEVPKRAKDLATIMAIGRANPPNAVEQSTFPDFYFRVTNNEHLVGLMKKFQCICEKTVIRRRHFVWDEEIL
NANPCLRTHMEPSFNVRQKIAVAEIPKMGAEAASRAIKEWGQPKSRITYLIFYTMSGMDLPVTDYKLTRILNLNLNVYQV
MLYQWDCIAGGTILCWAKCSAESQKGTRVLVVSSETTTVLVRAPSEDYHDDLVTQDLFGDGASALIVGADPDEEAKERPI
FTIVSTSQVVLPDSDGVIGGHLSEGGLMATLHRDVALIISKNVSKCLEEAFVTLGISDWNSIFWAPHPCRRAILDQVEER
VGLKPEKLWASRHVLAEFGNMTSVCVHFVLDEIHNRSTQGKTTTGEGFDWGVLFGFGPGLIVETVILRSVPLN
SEQ ID NO: 020
XP_020591420.1 [Phalaenopsis equestris]
MPNMESIKKEDGLATIMAIGRALPPNSIDQNSFPDFYFRVHNSEHLMDLKNKFRRICERTAIRKRHFVWNEEVLKQNPCL
RTFMEPSLNTRQEIVCSEIPKLGAEAARNAIREWGQPERSITHLIFCTTSGMNLPGADFEAAQILGLNHSVERVMLYQQG
CFAGGTVLRLAKCLAESRRGARVLVICAESTTSLVRSPSREHQYDLIAQALFADGASALIIGTEPNAEAGERPIFSIFST
AQVTLPDSGDAIRGYLKEGGLIATLAKDVPLIISENIERCLQEAFGPLGISDWNSIFWAPHPGGRAILDGIEDKLGLKPE
KLWAARHVLAEYGNMSSVCVHYILDEMRRRDVKNGKAPTGDGPEWGVLFGFGPGLTVETVVLRRLFL
SEQ ID NO: 021
[Leersia perrieri] UniProtKB - A0A0D9WZ61
MAPVPANGAATATATTQEIRRAQRADGPATVLAIGTANPETYVSQDEYADYYFRITKSEHLPELKDKLRRICNKSGIDKR
FMYVNEDVMEAHPEFADRNQSSLNARVEIASKAVPELAAAASAKAIAEWGRPATDITHLIFSTYSGVKAPSGDRLLASLL
GLRPNVSRTTLSLHGCYGGGRALQLAKELAENNRGARVLVACAEMTLIAFYGPEVGCNDTIVGQALFGDGSGAVIVGADP
VDAAGERPLFEMAFASQTTVPDSEGAITMQHTKGGMDYHIGSGIPEMLAGNIERCLADAFDSIGVAADWKDLFWAVHPGG
RRILDLIEEALGLDNGAMASRQVLREFGNMSGTTVIFVLNELRRRFKANGAEGADWGALMAFGPGVTIETMLLRVAAGLK
GN
SEQ ID NO: 022
[Leersia perrieri] UniProtKB - A0A0D9W1R8
MGSQGEYYNKEGRGQAAILGIGSAVPPYELPQSSFPDYYFDVSNSNHRQDLKAKFAKICERTMIEKRCVYMSEEFLRSNP
SVTAYSSPSIDVRQRLTDATVPELGAAAARVAIADWGRPASEISHLVMCSTVSGCMPGADYEVVKLLGLPLSTKRCMMYH
IGCHGGGTVLRIAKDIAENNPGARVLVVCSEVISMALRGPSDSNMGNLVGQALFGDAGAAVVVGTDPVESCGERALFEMV
AAMQDIIPDTEEMVAKLREDGLLYSLHRDVPVHIEANIEAIVNKSGVGAKAADWNEDVFWLVHPGGRDILDRVARRLGLR
HDKVEVSREVMRKHGNTLSSCVVIAMEEMRRRSAERGMGTAGEGLEWGLLFGFGPGLTVETILLRAPLTHPPPCAHAAVR
PQ
SEQ ID NO: 023
[Marchantia polymorpha] Genbank Q516Y1
MSRSRLIAQAVGPATVLAMGKAVPANVFEQATYPDFFFNITNSNDKPALKAKFQRICDKSGIKKRHFYLDQKILESNPAM
CTYMETSLNCRQEIAVAQVPKLAKEASMNAIKEWGRPKSEITHIVMATTSGVNMPGAELATAKLLGLRPNVRRVMMYQQG
CFAGATVLRVAKDLAENNAGARVLAICSEVTAVTFRAPSETHIDGLVGSALFGDGAAAVIVGSDPRPGIERPIYEMHWAG
EMVLPESDGAIDGHLTEAGLVFHLLKDVPGLITKNIGGFLKDTKNLVGASSWNELFWAVHPGGPAILDQVEAKLELEKGK
FQASRDILSDYGNMSSASVLFVLDRVRERSLESNKSTFGEGSEWGFLIGFGPGLTVETLLLRALPLQQAERV
SEQ ID NO: 024
[Marchantia polymorpha subsp. ruderalis] Genbank A0A176WB84
MAPQAVDATCAAETASAVPMSRPRLIAQAVGPATVLAMGKAVPANIFEQATYPDFFFNITNSNDKPALKAKFQRICDKSG
IKKRHFYLDQKILESNPAMCSYMETSLNCRQEIAIAQVPKLAKEASLNAIKEWGRPKSEITHIVMATTSGVNMPGAELAT
AKLLGLRPNVRRVMMYQQGCFAGATVLRVAKDLAENNAGARVLAICSEVTAVTFRAPCETHIDGLVGSALFGDGAAAVIV
GSDPRPGIERPMYEMHWAGEMVLPDSDGAIDGHLTEAGLVFHLLKDVPGLITKNIGGFLKDTKNLVGASSWNELFWAVHP
GGPAILDQVEAKLELEKGKFQASRDILSDYGNMSSASVLFVLDRVRERSLESNKSTFGEGNEWGFLIGFGPGLTVETLLL
RALPMQQAESV
SEQ ID NO: 025
[Marchantia polymorpha] Genbank A0A2R6XE22
MAPQAVDATCAAETASAVPMSRPRLIAQAVGPATVLAMGKAVPANIFEQATYPDFFFNITNSNDKPALKAKFQRICDKSG
IKKRHFYLDQKILESNPAMCSYMETSLNCRQEIAIAQVPKLAKEASLNAIKEWGRPKSEITHIVMATTSGVNMPGAELAT
AKLLGLRPNVRRVMMYQQGCFAGATVLRVAKDLAENNAGARVLAICSEVTAVTFRAPCETHIDGLVGSALFGDGAAAVIV
GSDPRPGIERPMYEMHWAGEMVLPDSDGAIDGHLTEAGLVFHLLKDVPGLITKNIGGFLKDTKNLVGASSWNELFWAVHP
GGPAILDQVEAKLELEKGKFQASRDILSDYGNMSSASVLFVLDRVRERSLESNKSTFGEGNEWGFLIGFGPGLTVETLLL
RALPMQQAESV
SEQ ID NO: 026
[Marchantia paleacea] Genbank A0A2P0VPI9
MSRSRLIAQAVGPATVLAMGKAVPANVFEQATYPDFFFNITNSNDKPALKAKFQRICDKSGIKKRHFYLDQKILESNPAM
CTYMETSLNCRQEIAVAQVPKLAKEASTNAIKEWGRPKSEITHIVMATTSGVNMPGAELATAKLLGLRPNVRRVMMYQQG
CFAGATVLRVAKDLAENNAGARVLAICSEVTAVTFRAPSETHIDGLVGSALFGDGAAAVIVGSDPRPGIERPIYEMHWAG
EMVLPESDGAIDGHLTEAGLVFHLLKDVPGLITKNIGGFLKDTKNLVGASSWNELFWAVHPGGPAILDQVEAKLELEKGK
FQASRDILSDYGNMSSASVLFVLDRVRERSLESNKSTFGEGSEWGFLIGFGPGLTVETLLLRALPLQQAERV
SEQ ID NO: 027
[Plagiochasma appendiculatum] Genbank AHY39238
MAPQAVDAACAAEATPVIPTSRPRLIAQAVGPATVLAMGKAVPHNVFEQATYPDFFFNITKCNDKPTLKAKFQRICDKSG
IKKRHFYLDQKILESSPSMCTYMETSLNCRQEIAIAQVPKLAKEAAQVAIKEWGRPKSEITHIVMATTSGVNMPGAELAT
AKLLGLRPNVRRVMMYQQGCFAGATVLRVAKDLAENNAGARVLAICSEVTAVTFRAPCETHIDGLVGSALFGDGAAAVIV
GADPRPEIERPMFEMHWAGEMVLPESDGAIDGHLTEAGLVFHLLKDVPGLITKNIGGFLKDTKNLVGASTWNDLFWAVHP
GGPAILDQVEAKLELEKNKFQASRDILADYGNMSSASVLFVLDRVRERSLESNKYTFGEGSEWGFLIGFGPGLTVETLLL
RALPTEQAASA
SEQ ID NO: 028
[Plagiochasma appendiculatum] Genbank AIV42295
MAPQAVDAACAAEATPVIPTSRPRLIAQAVGPATVLAMGKAVPHNVFEQATYPDFFFNITKCNDKPTLKAKFQRICDKSG
IKKRHFYLDQKILESNPSMCTYMETSLNCRQEIAIAHVPKLAKEAAQVAIKEWGRPKSEITHIVMATTSGVNMPGAELAT
AKLLGLRPNVRRVMMYQQGCFAGATVLRVAKDLAENNAGARVLAICSEVTAVTFRAPCETHIDGLVGSALFGDGAAAVIV
GADPRPEIERPMFEMHWAGEMVLPESDGAIDGHLTEAGLVFHLLKDVPGLITKNIGGFLKDTKNLVGASTWNDLFWAVHP
GGPAILDQVEAKLELEKNKFQASRDILADYGNMSSASVLFVLDRVRERSLESNKYTFGEGSEWGFLIGFGPGLTVETLLL
RALPTEQAASA
SEQ ID NO: 029
[Hydrangea macrophylla] Genbank AAN76182
MATKSVAVEEMCKAQKAGGPATILAIGTAVPSNCYYQSEYPDFYFRVTKSDHLTDLKSKFKRMCDRSSIKKRYMHLTEEI
LKENPNMCSFAAPSIDGRQDIVVKEIPKLAKEAASKAIKEWGQPESNITHLVFCTTSGVDMPGCDYQLTRLLGLRPSIKR
LMMYQQGCHAGGTGLRLAKDLAENNKGARVLVVCSEMTVINFRGPSEAHMDSLVGQSLFGDGASAVIVGSDPDLSTEHPL
YQIMSASQIIVADSEGVIDGHLRQEGLTFHLRKDVPSLVSDNIENTLVEAFTPILMDSIDSIIDWNSIFWIAHPGGPAIL
NQVQAKVGLKEEKLRVSRHILSEYGNMSSACVFFIMDEMRKRSVEEGKGTTGEGLEWGVLFGFGPGFTVETIVLHSVPI
SEQ ID NO: 030
[Hydrangea macrophylla] Genbank AAN76183
MATKSVAVEEMCKAQKAGGPATILAIGTAVPSNCYYQSEYPDFYFRVTKSDHLTDLKSKFKRMCERSSITKRYMHLTEEI
LEENPNMCTFAAPSIDGRQDIVVKEIPKLAKEAASKAIKEWGQPKSNITHLVFCTTSGVDMPGCDYQLTRLLGLRPSIKR
LMMYQQGCHAGGTGLRLAKDLAENNKGARVLVVCSEMTVINFRGPSEAHMDSLVGQSLFGDGASAVIVGSDPDLSTEHPL
YQIMSASQIIVADSEGAIDGHLRQEGLTFHLRKDVPSLVSDNIENTLVEAFTPILMDSIDSIIDWNSIFWIAHPGGPAIL
NQVQAKVGLKEEKLRVSRHILSEYGNMSSACVFFIMDEMRKRSVEEGKGTTGEGLEWGVLFGFGPGFTVETIVLHSVPI
SEQ ID NO: 031
[Pinus sylvestris] Genbank Q02323
MGGVDFEGFRKLQRADGFASILAIGTANPPNAVDQSTYPDFYFRITGNEHNTELKDKFKRICERSAIKQRYMYLTEEILK
KNPDVCAFVEVPSLDARQAMLAMEVPRLAKEAAEKAIQEWGQSKSGITHLIFCSTTTPDLPGADFEVAKLLGLHPSVKRV
GVFQHGCFAGGTVLRMAKDLAENNRGARVLVICSETTAVTFRGPSETHLDSLVGQALFGDGASALIVGADPIPQVEKACF
EIVWTAQTVVPNSEGAIGGKVREVGLTFQLKGAVPDLISANIENCMVEAFSQFKISDWNKLFWVVHPGGRAILDRVEAKL
NLDPTKLIPTRHVMSEYGNMSSACVHFILDQTRKASLQNGCSTTGEGLEMGVLFGFGPGLTIETVVLKSVPIQ
SEQ ID NO: 032
[Pinus densiflora] Genbank BAA89667
MGGVDFEGFRKLQRADGFASILAIGTANPPNAVDQSTYPDYYFRITGNEHNTELKDKFKRICERSAIKQRYMYLTEEILK
KNPDVCAFVEVPSLDARQAMLAMEVPRLAKEAAEKAIHEWGQSKSGITHLIFCSTTTPDLPGADFEVAKLLGLHPSVKRV
GVFQHGCFAGGTVLRLAKDLAENNRGARVLVICSETTAVTFRGPSETHLDSLVGQALFGDGASALIVGADPIPQVEKACF
EIVRTSQTVVPNSDGAIGGKVREVGLTFQLKGAVPDLISANIENCLVEAFSQFKICDWNKLFWVVHPGGRAILDRVEAKL
NLDPTKLIPTRHVMSEYGNMSSACVHFILDETRKASLRNGCSTTGEGLEMGVLFGFGPGLTIETVVLKSVPLQ
SEQ ID NO: 033
[Arachis hypogaea] Genbank AAA96434.1
MVSVSGIRKVQRAEGPATVLAIGTANPPNCIDQSTYADYYFRVTNSEHMTDLKKKFQRICERTQIKNRHMYLTEEILKEN
PNMCAYKAPSLDAREDMMIREVPRVGKEAATKAIKEWGQPMSKITHLIFCTTSGVALPGVDYELIVLLGLDPCVKRYMMY
HQGCFAGGTVLRLAKDLAENNKDARVLIVCSENTAVTFRGPSETDMDSLVGQALFADGAAAIIIGSDPVPEVEKPIFELV
STDQKLVPGSHGAIGGLLREVGLTFYLNKSVPDIISQNINDALNKAFDPLGISDYNSIFWIAHPGGRAILDQVEQKVNLK
PEKMKATRDVLSNYGNMSSACVFFIMDLMRKRSLEEGLKTTGEGLDWGVLFGFGPGLTIETVVLRSVAI
SEQ ID NO: 034
[Vitis quinquangularis] Genbank AEZ00059.1
MASVEEIRNAQRAKGPATILAIGTATPDHCVYQSDYADYYFRVTKSEHMTELKKKFNRICDKSMIKKRYIHLTEEMLEEH
PNIGAYMAPSLNIRQEIITAEVPKLGKEAALKALKEWGQPKSKITHLVFCTTSGVEMPGADYKLANLLGLETSVRRVMLY
HQGCYAGGTVLRTAKDLAENNAGARVLVVCSEITVVTFRGPSEDALDSLVGQALFGDGSAAVIVGSDPDVSIERPLFQLV
SAAQTFIPNSAGAIAGNLREVGLTFHLWPNVPTLISENIEKCLTQAFDPLGISDWNSLFWIAHPGGPAILDAVEAKLNLD
KKKLEATRHVLSEYGNMSSACVLFILDEMRRKSLKGENGTTGEGLDWGVLFGFGPGLTIETVVLHSIPMITN
SEQ ID NO: 035
[Vitis vinifera] Genbank ABV82966.1
MASVEEIRNAQRAKGPATILAIGTATPDHCVYQSDYADYYFRVTKSEHMSELKKKFNRICDKSMIKKRYIHLTEEMLEEH
PNIGAYMAPSLNIRQEIITAEVPKLGKEAALKALKEWGQPKSKITHLVFCTASGVEMPGADYKLANLLGLETSVRRVMLY
HQGCYAGGTVLRTAKDLAENNAGARVLVVCSEITVVTFRGPSEDALDSLVGQALFGDGSAAVIVGSDPDVSIERPLFQLV
SAAQTFIPNSAGAIAGNLREVGLTFHLWPNVPTLISENVEKCLTQAFDPLGISDWNSLFWIAHPGGPAILDAVEAKLNLD
KKKLEATRHVLSEYGNMSSACVLFILDEMRKKSHKGEKATTGEGLDWGVLFGFGPGLTIETVVLHSIPMVTN
SEQ ID NO: 036
[Polygonum cuspidatum] Genbank AFP97666.1
MAASTDEMTKALTAATVLAIGTANPPNCYYQADFPDFYFRATNSDHLTHLKHKFKRICEKSMIEKRYLQLTEDILKENPN
IGAYEAPSLDVRHEIQVKGVAQLGKEAALKAMQEWGQPKSKITHLIVCCIAGVDMPGANYQLTKLLDLNSSVKRFMFYHL
GCYAGGTVLRLAKDIAENNKGARVLIVCSEMTPICFRGPSETHIDSMVGQAIFGDGAAAVIVGANPDLTVEEPIFELIST
AQTIIPESDGAIEGHLLEVGLSFQLYQNVPALISNSIGTCLSEAFTPLNISNWNSLFWIAHPGGPAILDHVEATVGLNKE
KLKATRQVLNDYGNMSSACVFFIMDEMRKKSLENGHATTGEGLQWGVLFGFGPGITVETVVLRSVPII
SEQ ID NO: 037
[Psilotum nudum] Genbank BAA87924.1
MVSGEANGTSYQVRRGQSADGPATVLAIGTANPPNVFEQNSYPDFYFNVTNNTHKDELKAKFQRMCNRSGIRKRYLSFTE
ETLKANPSMGIYWEPSLDVRQDILAVEVPKLAKQASLNAIKEWGQPISNITHLVFCTTGPVSPGADAALMQMLGLNPSVK
RVLLYMQGCFAGGTVLRHAKDLAENNKGARVLVVCSESTAVTFRGPHENHLDNLVGQALFADGAAALIVGSDPIPNVEKA
WFEISLAESYLIPDSSPAIAGHLKEVGLEFHLTRDVSPVISKNILKILQDAFDGTGISDWNDVFIIAHPGGPAILDVIEE
KLKLVPEKLQASRHVLSEFGNMSSATVHFILDHMRKSSVEKGCATTGEGYQLGILLGLGPGMTVESIVLKSVPIASLLSS
SEQ ID NO: 038
[Morus notabilis] Genbank AOA48577.1
MAPTNGFVEESQTVIPRAGPAVASILAIGTSNPSNYFNQAEYADYYFRVTNSEHMTELKEKFKRICEKSLIKKRHMRLTE
DILKANPSICTYDGPSINERMDLKIVEMPKLGESAAIEALKEWGQPKSKITHIIVNSTSGVDMPGADYQLIRSLGLKTSV
KRVMLYHQGCFAGGTVLRIAKDLAENNPGARVLVVCSELTIPTFRGPSEEDSASLVGQAIFADGGSAVIVGANVPDEGSV
ERPLFRLVSNSQVILPNSENTVGGHLRDCGLTIVLSPEVPKLIGKNILTCLEEAFTPFGISDWNSLFWVPHPGGAAILRA
IEEKAELKKEKLKDTWNVWSEYGNMSSATVFFILNQMRKRSLEEKKSTTGDGLEWGVLLGFGPGLTVETVVLQSVPIVA
SEQ ID NO: 039
[Morus notabilis] Genbank AOA48578.1
MAPNNVSVEGSQPVIRRGGPGVASILAIGTANPDNFFNQADYPDYYFRVTNSEDKTELKEKFKRICEKSLIKKRHMRLTE
DILKENPSMCSYDAPSLNARMDLKLVEMPKLGESAAIAAIKEWGQPKSKITHLIVNSTSGVDMPGADYQLIKSLGLERSV
KRVMLYHQGCFAGGTVLRIAKDLAENNPGSRVLVVCSELTIPTFRGPSEDDSASLVGQAIFADGASAVIVGANVPDEGSV
ERPLFRLVSTSEVILPNSENTVGGHLRDCGLTIVLSPQVPKIIGKNIQTCLEEALGPFGISDWNSVFWAPHPGGAAIIKE
IEDKAGLEKEKLKDTWNVWSEYGNMSSATVFFILNQMRKRSLEEKKSTTGDGLEWGVLLGFGPGLTVETVVLQSVPIVA
SEQ ID NO: 040
[Psilotum nudum] Genbank BAA87925.1
MTTGEASLMKNGPASRARREERADGPATELAIGTANPSNVFDQETYPDFYFDVTNNTDKPELKAKFQRMCNSLESKKVHV
LHGGDVEGQPSMVFIGRILLDVRQGRRSRASAKLAKEASLKALREWGQPNSKITHLVFCTTAPVTLPGVDAALIQSLGLN
PSVKRVLLYMQGCFAGGTVLRHAKDLAENNRGARVLVVCSETTAVTFRGPHENHLDNLVGQALFADGASALIVGSDPISD
LEKPWFEIRWAGSYLIPESGQAIAGQLKEVGLEFHLTRDVSGLVSKNIVTILNEAFEGTGITDWNDIFIIPHPGGPAILD
VIQDRLKLQPEKLQASRHVLAEFGNMSSATVHFSLDQMRRSSVEKGCSTTGEGYELGILLGLGPGMTVESILLKSVPTWT
VAS
SEQ ID NO: 041
[Malus domestica] Genbank AFX71921.1
MAPLVKNHGEHQHAKILAIGTANPPNVYYQKDYPDFLFRVTKNEHRTDLREKFDRICEKSRTRKRYLHLTEEILKANPSI
YTYGAPSLDVRQDMLNPEVPKLGQQAALKAIKEWGQPISKITHLIFCTASCVDMPGADFQLVKLLGLNPSVTRTMIYEAG
CYAGATVLRLAKDFAENNEGARVLVVCAEITTVFFHGLTDTHLDILVGQALFADGASAVIVGANPEPKIERPLFEIVACR
QTIIPNSEHGVVANIREMGFTYYLSGEVPKFVGGNVVDFLTKTFEKVDGKNKDWNSLFFSVHPGGPAIVDQVEEQLGLKE
GKLRATRHVLSEYGNMGAPSVHFILDDMRKKSIEEGKSTTGEGLEWGVVIGIGPGLTVETAVLRSESITC
SEQ ID NO: 042
[Malus domestica] AFX71922.1
MAPLVKNEPQHAKILAIGTANPPNVYHQKDYPDFLFRVTKNEHRTDLREKFDRICEKSRTKKRYLHLTEEMLKANPNIYT
YGAPSLDVRQDICNIEVPKLGQEAALKAIKEWGQPISRITHLIFCTASCVDMPGCDFQLIKLLGLDPSVTRTMIYEAGCY
AGATVLRMAKDFAENNKGARVLVVCAEITTVFFHGLTDTHLDILVGQALFADGASAVIVGANPEPEIERPLFEIVACRQT
ILPNSEHGVVANIREMGFNYYLSGDVPKFVGGNVVDFMTKTFEKVDGKKKDWNSLFFSVHPGGPAIVDQVEEKLGLKEGK
LRATRHVLSEYGNMGAPTVHFILDEMRNKSIEEGKTTTGEGLEWGVVIGIGPGLTVETAVLRSESIRC
SEQ ID NO: 043
[Sorbus aucuparia] ABB89212.1
MAPLVKNHGEPQHAKILAIGTANPPNVYYQKDYPDFLFRVTKNEHRTDLREKFDRICEKSRTRKRYLHLTEEILKANPSI
YTYGAPSLDVRQDMLNSEVPKLGQQAALKAIKEWGQPISKITHLIFCTASCVDMPGADFQLVKLLGLNPSVTRTMIYEAG
CYAGATVLRLAKDFAENNEGARVLVVCAEITTVFFHGLTDTHLDILVGQALFADGASAVIVGANPEPKIERPLFEIVACR
QTIIPNSEHGVVANIREMGFTYYLSGEVPKFVGGNVVDFLTKTFEKVDGKNKDWNSLFFSVHPGGPAIVDQVEEQLGLKE
GKLRATRHVLSEYGNMGAPSVHFILDDMRKKSIEEGKSTTGEGLEWGVVIGIGPGLTVETAVLRSESIPC
SEQ ID NO: 044
[Pyrus communis] ANT48448.1
MAPLVKNHVEPPHAKILAIGTANPPNVYYQKDYPDFLFRVTKNEHRTDLREKFDRICEKSRTRKRYLYLTEEILNANPSI
YTYGAPSLDVRQDMLNPEVPKLGQQAALKAIKEWGQPISKITHLIFCTASCVDMPGADFQLVKLLGLNPSVTRTMIYEAG
CYAGATVLRLAKDFAENNEDARVLVVCAEITTVFFHGLTDTHLDILVGQALFADGASAVIVGANPEPEIERPLFEIVACR
QTIIPNSEHGVVANIREMGFNYYLSGDVPKFVGGSVVDFLTKTFEKVDGKNKDWNSLFLSVHPGGPAIVDQVEEQLGLKE
GKLRATRHVLSEYGNMGAPSVHFILDEMRNKSIGEGKATTGEGLEWGVVIGIGPGLTVETAVLRSESIPC
SEQ ID NO: 045
[Pyrus communis] ANT48449.1
MAPLVKNHVEPPHAKILAIGTANPPNVYYQKDYPDFLFRVTKNEHRTDLREKFDRICEKSRTRKRYLYLTEEILNANPSI
YTYGAPSLDVRQDMLNPEVPKLGQQAALKAIKEWGQPISKITHLIFCTASCVDMPGADFQLVKLLGLNPSVTRTMIYEAG
CYAGATVLRLAKDFAENNEDARVLVVCAEITTVFFHGLTDTHLDILVGQALFADGASAVIVGANPEPEIESPLFEIVACR
QTIIPNSEHGVVANIREMGFNYYLSGEVPKFVGGNVVDFLTKTFEKVDGKNKDWNSLFFSVHPGGPAIVDQVEEQLGLKE
GKLRATRHVLSEYGNMGAPSVHFILDEMRKKSIEEGKATTGEGLEWGVVIGIGPGLTVETAVLRSESIPC
SEQ ID NO: 046
[Phalaenopsis hybrid cultivar] CAA56277.1
MPSLESIKKAPRADGFASILAIGRANPDNIIEQSAYPDFYFRVTNSEHLVDLKKKFQRICEKTAIRKRHFVWNEEFLTAN
PCFSTFMDKSLNVRQEVAISEIPKLGAKAATKAIEDWGQPKSRITHLIFCTTSGMDLPGADYQLTQILGLNPNVERVMLY
QQGCFAGGTTLRLAKCLAESRKGARVLVVCAETTTVLFRAPSEEHQDDLVTQALFADGASAVIVGADPDEAADERASFVI
VSTSQVLLPDSAGAIGGHVSEGGLLATLHRDVPQIVSKNVGKCLEEAFTPFGISDWNSIFWVPHPGGRAILDQVEERVGL
KPEKLSVSRHVLAEYGNMSSVCVHFALDEMRKRSANEGKATTGEGLEWGVLFGFGPGLTVETVVLRSVPL
SEQ ID NO: 047
[Phalaenopsis hybrid cultivar] CAA56276.1
MLSLESIKKAPRADGFASILAIGRANPDNIIEQSAYPDFYFRVTNSEHLVDLKKKFQRICEKTAIRKRHFVWNEEFLTAN
PCFSTFMDKSLNVRQEVAISEIPKLGAKAATKAIEDWGQPKSRITHLIFCTTSGMDLPGADYQLTQILGLNPNVERVMLY
QQGCFAGGTTLRLAKCLAESRKGARVLVVCAETTTVLFRAPSEEHQDDLVTQALFADGASAVIVGADPDEAADERASFVI
VSTSQVLLPDSAGAIGGHVSEGGLLATLHRDVPQIVSKNVGKCLEEAFTPFGISDWNSIFWVPHPGGRAILDQVEERVGL
KPEKLSVSRHVLAEYGNMSSVCVHFALDEMRKRSANEGKATTGEGLEWGVLFGFGPGLTVETVVLRSVPL
SEQ ID NO: 048
[Bromheadia finlaysoniana] CAA10514.1
MASQVSPPSINMAPKADGFASILAIGRANPKNFIEQSTFPDFFFRVTNTEHMVDLKKKFQRICDKTSIRKRHFIWNEELL
TANPSLCTFMGNSLNLRHEVAVREIPKLGAEAATKAIQEWGQPKSFITHLVFCTTSGMDLPGADYQLTQILGLNLDIERV
MLHQQGCFLGGTTLRLAKYLAESRKGARVLVVCAETTTEFFRAPSEEHQEDLVTQSLFGDGASALIVGADPHEGARERAS
FILVSSSQVLLANSAHAITGHVSEGGIKATLHRDVPQIISNNLGKCLEEAFTPLGISDWNSIFWVLHPGGRAILDQVEEK
MGLEPEKLLISRHVLLEYGNMSSVCVHFALDEMRKRSSNEGKATTGEGLEWGVLFGFGPGLTIETVVLRSVSIS
SEQ ID NO: 049
[Dendrobium catenatum] PKU78204.1
MPSLESIKKAPRADGFASILAIGRANPENFIEQSAYPDLFFRITKSEHLVDLKNKFKRICDKTAIRKRHFVWTEEFITAN
PCFSTFMEKSLNIRQEVAIREIPKLGAEAAAKAIQEWGQPKSRITHLIFCTRSGMGLPGPDYQLTQILGLNPNVERVMIY
QQGCFAGGTTLRLAKCLAESHKGARVLVVCAETSTVLFRAPSMEHQEDLVTQALFADGASALIVGADPDETADEHASFVI
VSTSQVLLPESAGAIGGHVSEGGFLPMIHRDVPQIVSKNIGKCLEEAFTPLGIMDWNSIFWVPHPGGRAILDQLDERVGL
KPEKLFISRHVLKEYGNMSSASVHFALDEMRKWSAKEGTTGEGLEWGVLFGFGPGVTVDTVVLRSVPI
SEQ ID NO: 050
Olivetolic Acid Cyclase (OAC) from Cannabis sativa
MAVKHLIVLKFKDEITEAQKEEFFKTYVNLVNIIPAMKDVYWGKDVTQKNKEEGYTHIVEVTFESVETIQDYIIHPAHVG
FGDVYRSFWEKLLIFDYTPRK
SEQ ID NO: 051
Prenyltransferase from Streptomyces antibioticus
MSGAADVERVYAAMEEAAGLLGVTCAREKIYPLLTEFQDTLTDGVVVFSMASGRRSTELDFSISVPTSQGDPYATVVDKG
LFPATGHPVDDLLADTQKHLPVSMFAIDGEVTGGFKKTYAFFPTDDMPGVAQLSAIPSMPSSVAENAELFARYGLDKVQM
TSMDYKKRQVNLYFSELSEQTLAPESVLALVRELGLHVPTELGLEFCKRSFSVYPTLNWDTGKIDRLCFAVISTDPTLVP
STDERDIEQFRHYGTKAPYAYVGENRTLVYGLTLSPTEEYYKLGAYYHITDIQRRLLKAFDALED
SEQ ID NO: 052
THCA synthase (THCAS) from Cannabis sativa
MNCSAFSFWFVCKIIFFFLSFHIQISIANPRENFLKCFSKHIPNNVANPKLVYTQHDQLYMSILNSTIQNLRFISDTTPK
PLVIVTPSNNSHIQATILCSKKVGLQIRTRSGGHDAEGMSYISQVPFVVVDLRNMHSIKIDVHSQTAWVEAGATLGEVYY
WINEKNENLSFPGGYCPTVGVGGHFSGGGYGALMRNYGLAADNIIDAHLVNVDGKVLDRKSMGEDLFWAIRGGGGENFGI
IAAWKIKLVDVPSKSTIFSVKKNMEIHGLVKLFNKWQNIAYKYDKDLVLMTHFITKNITDNHGKNKTTVHGYFSSIFHGG
VDSLVDLMNKSFPELGIKKTDCKEFSWIDTTIFYSGVVNFNTANFKKEILLDRSAGKKTAFSIKLDYVKKPIPETAMVKI
LEKLYEEDVGAGMYVLYPYGGIMEEISESAIPFPHRAGIMYELWYTASWEKQEDNEKHINWVRSVYNFTTPYVSQNPRLA
YLNYRDLDLGKTNHASPNNYTQARIWGEKYFGKNFNRLVKVKTKVDPNNFFRNEQSIPPLPPHHH
SEQ ID NO: 053
AEX88416.1 biphenyl synthase 2 [Malus domestica]
MAPSVKDQVESQHAKILAIGTANPPNVYYQEDYPDFLFRVTKNEHRIDLREKFDRICEKSRTRKRYLYLTEEILKANPSI
YTYGAPSLDVRQDMLNPEVPKLGQQAALKAIKEWGQPISKITHLIFCTASCVDMPGADFQLVKLLGLNPSVTRTMIYEAG
CYAGATVLRLAKDFAENNEGARVLVVCAEITTVFFHGLTDTHLDILVGQALFADGASAVIVGANPEPEIESPLFEIVACR
QTIIPNSEHGVVANIREMGFNYYLSGEVPKFVGGNVVDFLTKTFEKVDGKNKDWNSLFFSVHPGGPAIVDQVEEQLGLKE
GKLRATRHVLSEYGNMGAPSVHFILDEMRKKSIEEGKATTGEGLEWGVVIGIGPGLTVETAVLRSEFITY
SEQ ID NO: 054
AEX88418.1 biphenyl synthase 4 [Malus domestica]
MAPLVKNQVEPQHAKILAIGTANPPNVYYQEDYPDFLFRVTKNEHRTDLREKFDRICEKSRTKKRYLHVTEEILQANPSI
YTYGAPSLDVRQDMLNPEVPKLGQQAALKAIKEWGQPISKITHLIFCTASCVDMPGADFQLVKLLGLNPSVTRTMIYEAG
CYAGATVLRLAKDFAENNEGARVLVVCAEITTVFFHGLTDTHLDILVGQALFADGASAVIVGANPEPEIERPLFEIVACR
QTIIPNSEHGVVAHIREMGFEYYLSGEVPKFVGGNVVDFLTKTFEKVDGKKKDWNSLFYSVHPGGPAIVDQVEEQLGLKE
GKLRATRHVLSEYGNMGAPSVHFILDEMRNKSIEEGKSTTGEGLEWGVVIGIGPGLTVETIVLRSESIACEKLA
SEQ ID NO: 055
AAU43217.1 chalcone synthase [Arachis hypogaea]
MVNVSEIRKAQRAEGPATIMAIGTATPQNCVDQSTYPDYYFRTTNSQHMTELKEKFQRMCDKSMIKKRYMYLTEEILKEN
PNMCKYMAPSLDARQDMVVVEVPRLGKEAATKAIKEWGQPKSKITHLIFCTTSGVDMPGADYQLTKLLGLRPSVKRYMMY
QQGCFAGGTVLRLAKDLAENNKGARVLVVCSEITAVTFRGPSETHLDSLVGQALFGDGAAALIVGSDPLPQIEKPVFELV
WTAQTLAPDSEGAIDGHLREVGLTFHLLKDVPGIVSKNINKALTEAFDPLGISDYNSIFWIAHPGGPAILDQVEDKLKLK
PHKLRATRDVLSDYGNMSSACVLFILDHMRNKSLQQGLQTTGEGLEWGVLFGFGPGLTIETVVLHSVAI
SEQ ID NO: 056
AAL49965.1 chalcone synthase 8 [Sorghum bicolor]
MTTGKVTLEAVRKAQRAEGPATVLAIGTATPANCVYQADYPDYYFRVTKSEHLTDLKEKFKRICHKSMIRKRYMHLTEDI
LEENPNMSSYWAPSLDARQDILIQEIPKLGAEAAEKALKEWGQPRSRITHLVFCTTSGVDMPGADYQLIKLLGLCPSVNR
AMMYHQGCFAGGMVLRLAKDLAENNRGARVLIVCSEITVVTFRGPSESHLDSLVGQALFGDGAAAVIVGADPSEPAERPL
FHLVSASQTILPDSEGAIEGHLREVGLTFHLQDRVPQLISMNIERLLEDAFAPLGISDWNSIFWVAHPGGPAILNMVEAK
VGLDKARMCATRHILAEYGNMSSVCVLFILDEMRNRSAKDGHTTTGEGMEWGVLFGFGPGLTVETIVLHSVPITTVAA

### EXAMPLES

Methods for Olivetolic Acid Production in E. coli

Unless otherwise specified, the Examples provided herein were performed according to the following methods.

To test olivetolic acid production in E. coli, strains comprising a putative OLS gene and an OAC gene from C. sativa were inoculated in multi-well plates containing LB supplemented with 1% glucose and appropriate concentrations of antibiotics. After 6 hours of cultivation at 32° C., the cells were transferred with a 10% inoculum to a P-limited seed medium (see Table 2) and cultivated for ˜18 hours at 32° ° C. to reach an OD of 2.0-4.0. The cultures were transferred with a 20% inoculum in a P-minimal medium (see Table 3) supplemented with 2% glucose and appropriate concentrations of antibiotics. After 3 hours of cultivation at 32° C., the culture was spiked with 4 mM hexanoic acid (˜OD 1.5-2.0). The resulting cultures were then harvested at either 3 hours or 21 hours post hexanoic acid spike; a final OD was taken. 300 μL of butyl acetate containing 500 mg/L undecanoic acid was added to each multi-well plate. The plates were then vortexed for 30 minutes at 1500 rpm and centrifuged for 10 minutes at 4500 rpm. 50 μL of organic layer was transferred to a 96-well plate and derivatized with 50 μL of N,O-Bis(trimethylsilyl)trifluoroacetamide (BSTFA). The plate was then incubated at room temperature for 2 hours to allow for complete derivatization. The samples were then run on GC-FID for analytical quantification of olivetolic acid (OLA), olivetol (OL), PDAL, and hexanoic acid.

TABLE 2
P-Limited Seed Media

| Component | Stock Concentration | | Final Concentration | |
|---|---|---|---|---|
| Water | 100% | | | |
| TVS (Trace Vitamins) | 25000 | X | 1 | X |
| Thiamine | 1 | g/L | 0.1 | mg/mL |
| Biotin | 40 | mM | 0.25 | mM |
| Antibiotic(s) | 1000 | X | 1 | X |
| MgSO₄ | 1M | | 1 | mM |
| Trace metals | 1000 | X | 1 | X |
| (w/selenium) | | | | |
| Citric Acid | 100 | g/L | 0.1 | g/L |
| Ferrous Sulfate | 10 | g/L | 0.01 | g/L |
| Bis-Tris, pH 7.0 | 2M | | 200 | mM |
| Glucose | 500 | g/L (50%) | 10 | g/L |
| P-limited Salt Solution | 1.06 | g/L KH₂PO₄ | 0.424 | g/L KH₂PO₄ |
| w/(NH₄)₂SO₄ | 0.94 | g/L Na₂HPO₄ | 0.376 | g/L Na₂HPO₄ |
| | 25 | g/L (NH₄)₂SO₄ | 10 | g/L (NH₄)₂SO₄ |
| | 5 | g/L NaCl | 2 | g/L NaCl |

TABLE 3
P-Limited Production Media

| Component | Stock Concentration | | Final Concentration | |
|---|---|---|---|---|
| Water | 100% | | | |
| TVS (Trace Vitamins) | 25000 | X | 1 | X |
| Thiamine | 1 | g/L | 0.1 | mg/mL |
| Biotin | 40 | mM | 0.25 | mM |
| Antibiotic(s) | 1000 | X | 1 | X |
| MgSO₄ | 1M | | 1 | mM |
| TM-G (w/selenium) | 1000 | X | 1 | X |
| Citric Acid | 100 | g/L | 0.1 | g/L |
| Ferrous Sulfate | 10 | g/L | 0.01 | g/L |
| Triton | 10% | | 0.0125% | |
| Glucose | 500 | g/L (50%) | 20 | g/L |
| Bis-Tris, pH 7.0 | 2M | | 200 | mM |
| P-limited Salt Solution | 1.06 | g/L KH₂PO₄ | 0.318 | g/L KH₂PO₄ |
| w/(NH₄)₂SO₄ | 0.94 | g/L Na₂HPO₄ | 0.282 | g/L Na₂HPO₄ |
| | 25 | g/L (NH₄)₂SO₄ | 7.5 | g/L (NH₄)₂SO₄ |
| | 5 | g/L NaCl | 1.5 | g/L NaCl |

Olivetol, PDAL, OLA, HTAL, CBGA and combinations thereof were analyzed by LCMS or LCMS/MS methods using C18 reversed phase chromatography coupled to either EXACTIVE™ (ThermoFisher) or QTRAP® 4500 (Sciex) mass spectrometers.

For reversed phase LCMS, compounds were identified by their LC retention times and MRM transitions specific to the compounds. LCMS/MS analysis was conducted on a Shimadzu UHPLC system coupled with AB Sciex QTRAP 4500 mass spectrometer. Agilent Eclipse XDB C18 column (4.6×3.0 mm, 1.8 μm) was used with a 1-min gradient elution at 1 mL/min using water containing 0.1% ammonia acetate as mobile phase A and 90% methanol containing 0.1% ammonia acetate as mobile phase B. The LC column temperature was maintained at 45° C. Negative ionization mode was used for all the analytes.

### Example 1. Plasmid Expression in E. coli

The genes for 20 Type-III PKS enzymes were codon optimized for E. coli and cloned under control of a constitutive promoter into an expression vector (p15a replicon, carbenicillin resistance marker). The 20 plasmids were transformed into an E. coli derivative strain which overexpressed an acyl-CoA synthetase (fadD) gene and expressed an olivetolic acid cyclase (OAC) from Cannabis sativa.

The in vivo assay procedure and analytical methods were performed as described above.

The results in Table 4 show that strains expressing several of the Type-III PKS enzymes produced the same products as the ones expressing the olivetol synthase (OLS) from C. sativa, namely olivetol (OL), olivetolic acid (OLA) and pentyl diacetic acid lactone (PDAL). The results indicate these polyketide synthases have relaxed substrate specificity and can accept hexanoyl-CoA as starter substrates and thereby enable the production of olivetolic acid and related products.

The strains expressing seven Type-III PKS enzymes produced more olivetolic acid than the strain expressing OLS from C. sativa. For example, the E. coli strains with the Type-III PKS enzymes QCO76957.1 from Dendrobium officinale, QDX46968.1 from Anoectochilus roxburghii, AAX54693.1 from Phalaenopsis hybrid cultivar, and AAZ32094.1 from Oncidium hybrid cultivar produced over two-fold higher levels of OLA than the E. coli strain with OLS from C. sativa.

TABLE 4
OL, OLA, and PDAL Production from Plasmids

| | | SEQ | OL | OLA | PDAL | Total |
|---|---|---|---|---|---|---|
| Organism | UniProt # | ID NO. | (μM) | (μM) | (μM) | (μM) |
| Cannabis sativa olivetol synthase | B1Q2B6 | 001 | 324 | 279 | 470 | 1072 |
| Dendrobium officinale | QCO76957.1 | 008 | 579 | 754 | 761 | 2095 |
| Anoectochilus roxburghii | QDX46968.1 | 006 | 597 | 823 | 536 | 1956 |
| Phalaenopsis hybrid cultivar | AAX54693.1 | 007 | 606 | 653 | 615 | 1874 |
| Oncidium hybrid cultivar | AAZ32094.1 | 002 | 672 | 751 | 358 | 1781 |
| Paphiopedilum purpuratum | AFU07719.1 | 014 | 289 | 302 | 815 | 1406 |
| Dendrobium catenatum | XP_020704098.1 | 004 | 432 | 390 | 250 | 1072 |
| Cymbidium hybrid cultivar | AIM58716.1 | 003 | 427 | 436 | 181 | 1045 |
| Apostasia shenzhenica | PKA53998.1 | 015 | 268 | 256 | 481 | 1005 |
| Paphiopedilum micranthum | AFU07718.1 | 013 | 159 | 152 | 638 | 948 |
| Phalaenopsis equestris | XP_020591420.1 | 020 | 54 | 36 | 378 | 468 |
| Bletilla striata | AHH25569.1 | 009 | 93 | 98 | 129 | 320 |
| Dendrobium catenatum | XP_020704102.1 | 011 | 26 | 19 | 102 | 148 |
| Bromheadia finlaysoniana | CAA10514.1 | 012 | 0 | 0 | 61 | 61 |
| Apostasia shenzhenica | PKA56805.1 | 017 | 27 | 10 | 0 | 37 |
| Leersia perrieri | A0A0D9WZ61 | 021 | 34 | 0 | 0 | 34 |
| Oncidium Gower Ramsey | AAZ32093.1 | 010 | 16 | 0 | 0 | 16 |
| Paphiopedilum armeniacum | AFU07709.1 | 016 | 0 | 0 | 0 | 0 |
| Paphiopedilum × areeanum | AFU07710.1 | 019 | 0 | 0 | 0 | 0 |
| Dendrobium catenatum | PKU71375.1 | 018 | 0 | 0 | 0 | 0 |
| Phalaenopsis equestris | XP_020572025.1 | 005 | 0 | 0 | 0 | 0 |
| Leersia perrieri | A0A0D9W1R8 | 022 | 0 | 0 | 0 | 0 |

### Example 2. E. coli Genome Integration

Three of the type-III PKS genes from Example 1 were integrated under control of a constitutive promoter into an E. coli derivative strain which overexpressed an acyl-CoA synthetase (fadD) gene and expressed an olivetolic acid cyclase (OAC) from Cannabis sativa.

The in vivo assay procedure and analytical methods were performed as described above.

The results in Table 5 show that strains with the chromosomally expressed type-III PKS enzymes QCO76957 from Dendrobium officinale, QDX46968 from Anoectochilus roxburghii, AAZ32094 from Oncidium hybrid cultivar and AIM58716 from Cymbidium hybrid cultivar produced olivetol (OL), olivetolic acid (OLA) and pentyl diacetic acid lactone (PDAL). The results indicate these polyketide synthases have relaxed substrate specificity and can accept hexanoyl-CoA as starter substrates and thereby enable the production of olivetolic acid and related products.

TABLE 5
OL, OLA, and PDAL Production from Genome-Integrated Genes

| | | SEQ | OL | OLA | PDAL | Total |
|---|---|---|---|---|---|---|
| Organism | UniProt # | ID NO. | (μM) | (μM) | (μM) | (μM) |
| Dendrobium officinale | QCO76957.1 | 008 | 178 | 223 | 82 | 483 |
| Anoectochilus roxburghii | QDX46968.1 | 006 | 98 | 136 | 42 | 276 |
| Oncidium hybrid cultivar | AAZ32094.1 | 002 | 355 | 534 | 149 | 1038 |
| Cymbidium hybrid cultivar | AIM58716.1 | 003 | 249 | 472 | 206 | 927 |

### Example 3. Specific Activity Assay of Type-III Polyketide Synthases

In this Example, the specific activities of three previously uncharacterized type-III polyketide synthases (PKS) to form olivetolic acid from hexanoyl-CoA and malonyl-CoA in the presence of olivetolic acid cyclase (OAC) were determined and compared to the olivetol synthase (OLS) from C. sativa.

Assays were performed in a total volume of 50 μL in 100 mM Tris, pH 7.5 buffer containing 100 μM malonyl-CoA; 100 UM hexanoyl-CoA; a malonyl-CoA regenerating system comprising malonyl-CoA synthetase, excess malonate, and ATP; and excess purified olivetolic acid cyclase (OAC). The major product in this assay was olivetolic acid (OLA), and the triketide pentyl diacetic acid lactone (PDAL) was a side product. N-His-tagged purified enzymes were assayed under these assay conditions at enzyme concentrations and reaction times suitable for initial rate measurements of product formation. Reactions were initiated by addition of the PKS, then incubated for 30 min. Subsequently, 10 μL of reaction solution was removed and quenched into 15 volumes of 75% acetonitrile containing 0.1% formic acid and internal standards, then centrifuged to pellet denatured protein. Supernatants were transferred to fresh plates for LC-MS analysis of OLA, olivetol (OL), and PDAL as described in the Method section above.

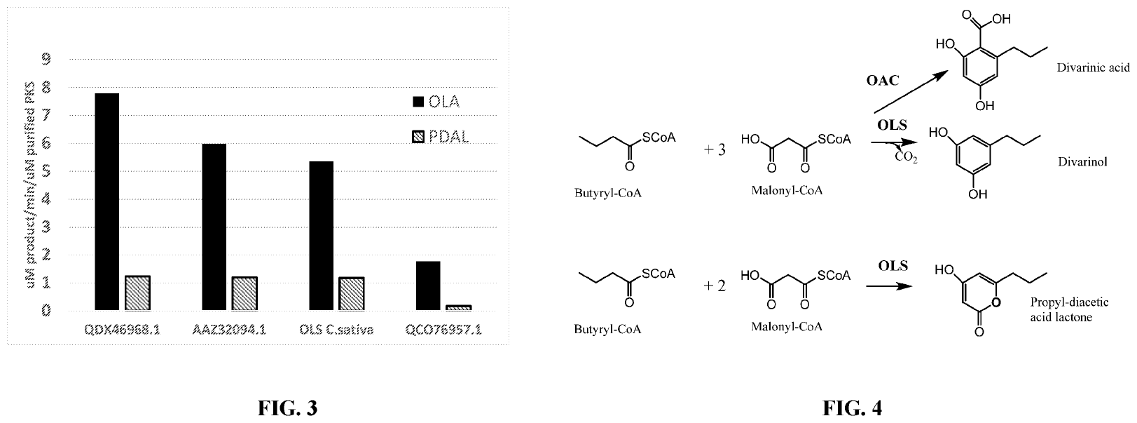

Results are shown in FIG. 3. As shown in FIG. 3, two of the unknown type-III polyketide synthases, QDX46968.1 from Anoectochilus roxburghii (SEQ ID NO:6) and AAZ32094.1 from Oncidium hybrid cultivar (SEQ ID NO:2), showed higher specific activities to form olivetolic acid from hexanoyl-CoA and malonyl-CoA in the presence of olivetolic acid cyclase (OAC) than olivetol synthase (OLS) from (′. sativa. This was a surprising result, because in contrast to olivetol synthase (OLS) from C. sativa, hexanoyl-CoA is not expected to be the native substrate and olivetolic acid is not expected to be the native product of these polyketide synthases.

### Example 4. Evaluation of Production Inhibition of Type-III Polyketide Synthases

In this Example, the ability of OLA, OL and PDAL products to inhibit the activity of two previously uncharacterized PKS and olivetol synthase (OLS) from (′. sativa were evaluated. Product inhibition is a common feature of biosynthetic enzymes.

Assays were performed as described in Example 3 with the following exception: instead of hexanoyl-CoA (C6), butyryl-CoA (C4) was used as substrate along with malonyl-CoA, in order to evaluate the inhibitory effect of the products with hexanoyl-CoA (i.e., OLA, OL and PDAL) on the activity of the enzymes. Accordingly, the products in this assay were the tetraketides divarinic acid (DVA) and divarinol (DVL) and the triketide propyl diacetic acid lactone (propyl-DAL) (see FIG. 4). The rates to form these products were measured in the presence of various concentrations of OLA, OL, and PDAL.

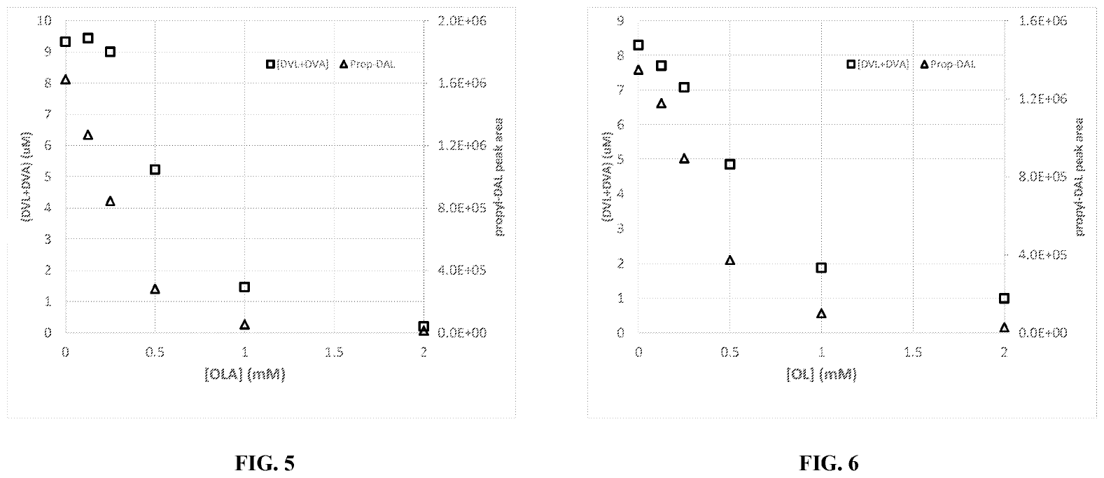

FIGS. 5, 6, and 7 show the impact of increasing concentrations of OLA, OL and PDAL, respectively, on the activity of OLS from C. sativa. As shown in FIGS. 5-7, all three products considerably inhibited the activity of OLS from (′. sativa. For example, FIG. 5 shows that at 1 mM OLA, the amount of DVA+DVL formed by the enzyme decreased from over 9 μM (formed in the absence of OLA) to 1.5 μM. FIG. 6 shows that at 1 mM OL, the amount of DVA+DVL decreased from over 8 μM (formed in the absence of OL) to 2 μM. At 2 mM OLA or OL, the enzyme was almost completely inactive (FIGS. 5 and 6). PDAL was also inhibitory, but to a somewhat lesser extent (FIG. 7). The results indicate that the OLS from (′. sativa is subject to significant inhibition by its native products.

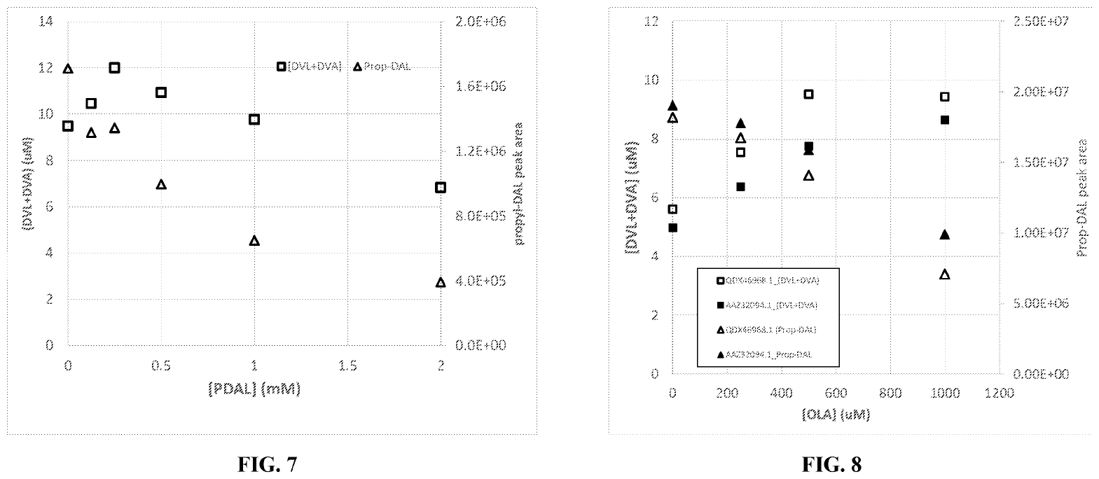

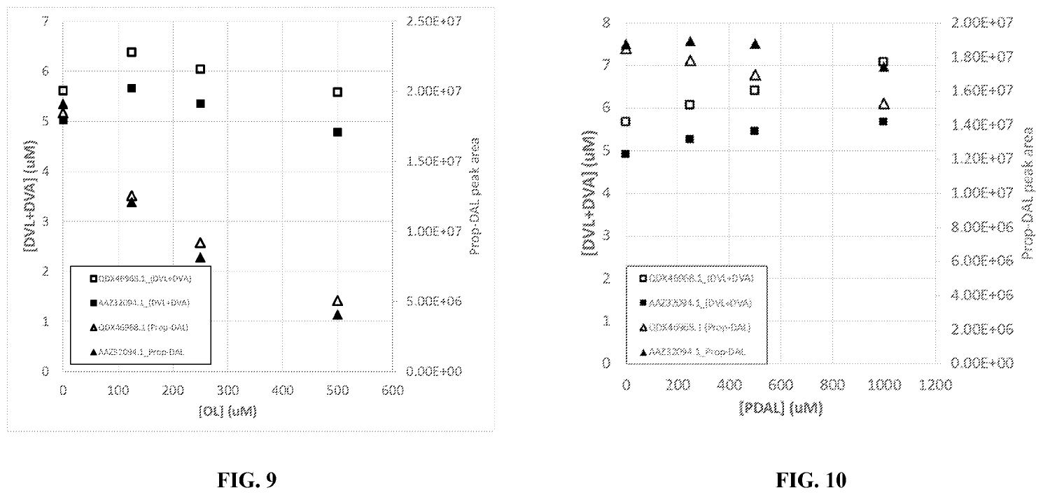

FIGS. 8, 9, and 10 show the impact of increasing concentrations of OLA, OL and PDAL, respectively, on the activity of QDX46968.1 from Anoectochilus roxburghii (SEQ ID NO:6) and AAZ32094.1 from Oncidium hybrid cultivar (SEQ ID NO:2). As shown in FIGS. 8-10, both enzymes showed a surprising behavior that was very different than the OLS from C. sativa. FIG. 8 shows that the formation of the tetraketide products (DVL+DVA) was not inhibited, but rather stimulated by OLA, whereas the triketide product propyl-DAL was inhibited by OLA. The net effect was that the presence of OLA increased the formation of the desired tetraketide product and decreased the undesired propyl-DAL derailment product. For example, FIG. 8 shows that at 1 mM OLA, the amount of DVA+DVL formed by both enzymes increased from 5-6 μM (formed in the absence of OLA) to about 9 μM while the amount of the triketide propyl-DAL decreased from about 9 μM (formed in the absence of OLA) to 3-5 μM. FIG. 9 shows that the formation of the tetraketide products (DVL+DVA) for both enzymes was not inhibited by OL, whereas the triketide product propyl-DAL was decreased in the presence of OL. FIG. 10 shows that the activity of both enzymes was not inhibited by PDAL.

### Example 5. Active Site Mutations of Olivetol Synthase from Anoectochilus Roxburghii

The OLS from Anoectochilus roxburghii (UniProt ID QDX46968.1; SEQ ID NO:6), designated as “OLS_Aro,” was subjected to mutagenesis and assayed for improved activity.

### Library Constructs and Strains

Single amino acid positions of interest of OLS_Aro were identified based on a 7 Å radial proximity around the active site predicted based on docking results with the tetraketide product into a structural homology model of OLS_Aro. Variants were constructed as a plasmid-based library using specific primers at the positions undergoing mutagenesis, amplifying fragments via Polymerase Chain Reaction (PCR), and circularizing plasmids via Gibson ligation. For site-saturation mutagenesis of selected amino acids sites, a compressed-codon approach was used to eliminate codon redundancy to lower library size. The plasmid-base used was the pZS*vector (Novagen) with expression of the OLS_Aro under control of a pAl promoter and lactose (lac) operator. Plasmids containing the variants of OLS_Aro were transformed into an E. coli host with known thioesterase genes deleted and plated onto agar plates with suitable antibiotic selection. Variants of interest were identified by activity assay described below and sequenced.

### Cell Cultures for OLS_Aro Variant Library

Single colonies were picked for growth in Luria Bertani (LB) medium in 384-well plates containing with carbenicillin (Carb). Cultures were grown overnight at 35° C. The following day cultures were diluted into fresh medium of LB containing carbenicillin and Isopropyl β-D-1-thiogalactopyranoside (IPTG). After 20 hours of growth and expression, cells were pelleted by centrifugation and media discarded. Cells pellets were stored at −20° C. until ready for assay. The number of samples screened was approximately three times the number of total possible variants.

### High-Throughput Activity Assay

Cell pellets were thawed then chemically lysed using B-PERII reagent in the presence of 1 mM DTT, benzonase, and lysozyme. Assays were performed in 384-well plates in a total volume of 50 μL in 100 mM Tris, pH 7.5 buffer containing 20 mM NH₄Cl, 100 UM malonyl-CoA, 20 μM hexanoyl-CoA or butyryl-CoA (CoALA), and a malonyl-CoA recycling system comprised of malonyl-CoA synthetase (1 μM), malonate (1 mM), MgCl₂ (5 mM), and ATP (1 mM). These enzymatic coupling reagents maintain malonyl-CoA in the assay with free CoA generated by OLS catalysis.

Reactions were initiated by addition of cell lysate, then incubated for 20 mins or 1 hr for hexanoyl-CoA and butyryl-CoA, respectively. Subsequently, 45 uLs of the reaction solution was quenched with 135 uLs of 75% acetonitrile containing 0.1% formic acid and internal standards. then filtered to remove precipitated protein. Filtrates analyzed by LC/MS for the quantification of olivetolic acid (OLA), olivetol (OL), and pentyl diacetic acid lactone (PDAL); or divarinic acid (DVA), divarinol (DVL), and propyl diacetic acid lactone (Propyl-DAL).

### Analysis of OLS Reaction Products

Olivetolic acid, olivetol, and PDAL or divarinic acid, divarinol, and Propyl-DAL were analyzed by Liquid Chromatography/Mass spectrometry (LC/MS) using C18 reversed phase chromatography coupled to either Exactive (Thermofisher) or Qtrap 4500 (Sciex) mass spectrometers. Compounds were identified by their LC retention times and Multiple reaction monitoring (MRM) transitions specific to the compounds. Negative ionization mode was used for all the analytes.

### Data

Under the screening conditions described above, products are detected in the low or sub μM range. For wild-type OLS_Aro reactions in the absence of Olivetolic acid Cyclase (OAC), the major products are OL and PDAL or DVL and Propyl-DAL; OLA and DVA are not significant. The desired product is OL or DVL, and the undesired (“derailment”) product is PDAL or Propyl-DAL. Two comparative measures were considered: 1) the total desired product improvements determined by (OLₘᵤₜ+OL_{WT}) or (DVLₘᵤₜ+DVL_{WT}), i.e., fold-improvement in OL or DVL; and 2) the improvements to the ratio of product-to-by-product relative to wild-type determined by (OL/PDAL)ₘᵤₜ+(OL/PDAL)_{WT} or (DVL/Propyl-DAL)ₘᵤₜ+(DVL/Propyl-DAL)_{WT}, i.e., fold-improvement in OL/PDAL or DVL/Propyl-DAL.

### Results

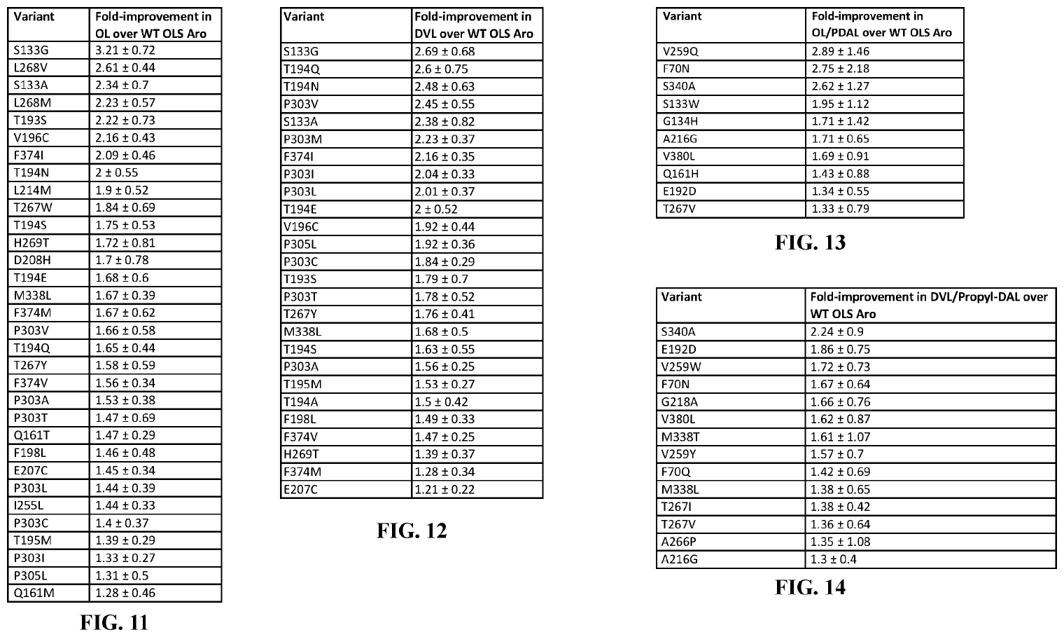

FIG. 11 shows the fold-improvement in olivetol production by the variants of OLS_Aro over wild-type OLS_Aro. FIG. 12 shows the fold-improvement in divarinol production by the variants of OLS_Aro over wild-type OLS_Aro. FIG. 13 shows the fold-improvement in the OL/PDAL ratio of the variants of OLS_Aro over wild-type OLS_Aro. FIG. 14 shows the fold-improvement in the DVL/Propyl-DAL ratio of the variants of OLS_Aro over wild-type OLS_Aro.

The results demonstrate that: 1) several sites and certain residues at these sites in OLS_Aro have the effect of increasing the rate of product formation of OL and DVL; and 2) several sites and certain residues at these sites in OLS_Aro have the effect of lowering byproduct formation.

What is claimed is:

1. A polynucleotide comprising: (a) a nucleic acid sequence encoding an olivetol synthase (OLS) of any of SEQ ID NOs:2-49; and (b) a heterologous regulatory element operably linked to the nucleic acid sequence.

2. The polynucleotide of claim 1, wherein the nucleic acid sequence encodes an OLS of SEQ ID NO:2, 3, 4, 6, 7, 8, 9, 11, 13, 14, 15, or 20.

3. The polynucleotide of claim 1, wherein the nucleic acid sequence encodes an OLS of SEQ ID NO:4, 6, 8, 9, 11, 13, 15, or 20.

4. The polynucleotide of claim 1, wherein the nucleic acid sequence encodes an OLS of SEQ ID NO:4, 6, 8, 9, 11, or 15.

5. The polynucleotide of claim 1, wherein the nucleic acid sequence encodes an OLS of SEQ ID NO:2, 3, 6, or 8.

6. The polynucleotide of claim 1, wherein the nucleic acid sequence encodes an OLS of SEQ ID NO:6 or 8.

7. The polynucleotide of claim 1, wherein the nucleic acid sequence encodes an OLS of SEQ ID NO:2.

8. The polynucleotide of claim 1, wherein the nucleic acid sequence encodes an OLS of SEQ ID NO:6.

9. The polynucleotide of any one of claims 1 to 8, wherein the OLS further comprises an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

10. The polynucleotide of claim 9, wherein the amino acid variation is an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

11. The polynucleotide of claim 10, wherein the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof.

12. The polynucleotide of claim 9, wherein the amino acid variation is an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

13. The polynucleotide of any one of claims 1 to 12, wherein the heterologous regulatory element comprises an Escherichia coli promoter.

14. A non-naturally occurring olivetol synthase (OLS) comprising at least 90% sequence identity to any of SEQ ID NOs:2-49, and further comprising an amino acid substitution at an amino acid position corresponding to position 82, 125, 126, 131, 185, 186, 187, 189, 190, 195, 197, 204, 208, 209, 210, 211, 239, 249, 250, 257, 314, 331, and/or 332 of SEQ ID NO:1.

15. A non-naturally occurring olivetol synthase (OLS) comprising at least 90% sequence identity to any of SEQ ID NOs: 10-45, SEQ ID NO:48, or SEQ ID NO:49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

16. A non-naturally occurring olivetol synthase (OLS) comprising at least 95% sequence identity to SEQ ID NO:2 or any of SEQ ID NO:4-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

17. The OLS of claim 15 or 16, wherein the amino acid variation is an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

18. The OLS of claim 17, wherein the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof.

19. The OLS of claim 15 or 16, wherein the amino acid variation is an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

20. A non-naturally occurring olivetol synthase (OLS) comprising at least 90% sequence identity to any of SEQ ID NOs:2-49, and further comprising an amino acid variation at an amino acid position corresponding to amino acid position 133, 134, 192, 193, 194, 196, 198, 214, 216, 218, 259, 266, 267, 268, 338, 340, and/or 380 of SEQ ID NO:6.

21. The OLS of claim 20, wherein the amino acid variation is an amino acid substitution, wherein the amino acid substitution comprises S133A, S133G, S133W, G134H, E192D, T193S, T194A, T194E, T194N, T194Q, T194S, V196C, F198L, L214M, A216G, G218A, V259Q, V259W, V259Y, A266P, T267I, T267V, T267W, T267Y, L268M, L268V, M338L, M338T, S340A, V380L, or a combination thereof, wherein the amino acid position corresponds to SEQ ID NO:6.

22. A non-naturally occurring olivetol synthase (OLS) comprising at least 90% sequence identity to any of SEQ ID NOs:2-49, and further comprising an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

23. The OLS of claim 22, wherein the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof.

24. The OLS of any one of claims 20 to 23, comprising at least 90% sequence identity to SEQ ID NO:2 or 6.

25. The OLS of any one of claims 16 to 24, comprising at least 97% sequence identity to SEQ ID NO:2 or 6.

26. The OLS of any one of claims 15 to 25, wherein the OLS produces at least 1.1-fold higher amount of olivetol and/or divarinol as compared to a wild-type counterpart of the OLS under the same reaction conditions.

27. The OLS of any one of claims 15 to 26, wherein a ratio of olivetol to pentyl diacetic acid lactone (OL:PDAL) production or a ratio of divarinol to propyl diacetic acid lactone (DVL:Propyl-DAL) production for the OLS is about 1.3-fold higher as compared to a wild-type counterpart of the OLS under the same reaction conditions.

28. A polynucleotide comprising a nucleic acid encoding the OLS of any one of claims 14 to 27.

29. The polynucleotide of claim 28, further comprising a heterologous regulatory element operably linked to the nucleic acid.

30. An expression construct comprising the polynucleotide of any one of claims 1 to 13, claim 28, or claim 29.

31. The expression construct of claim 30, wherein the expression construct is a bacterial expression construct.

32. An olivetol synthase (OLS) encoded by the polynucleotide of any one of claims 1 to 13.

33. The OLS of any one of claims 14 to 27 or claim 32, wherein activity of the OLS is not substantially inhibited by olivetolic acid, olivetol, pentyl diacetic acid lactone, or combination thereof.

34. The OLS of any one of claims 14 to 27, claim 32, or claim 33, wherein activity of the OLS is not substantially inhibited by olivetolic acid or olivetol.

35. The OLS of any one of claims 14 to 27 or any one of claims 32 to 34, wherein the OLS has a higher rate of production of olivetolic acid in the presence of hexanoyl-CoA, malonyl-CoA, and olivetolic acid cyclase (OAC) as compared to an OLS from C. sativa under the same reaction conditions.

36. The OLS of any one of claims 14 to 27 or any one of claims 32 to 35, wherein the OLS has a higher rate of production of olivetolic acid in the presence of hexanoyl-CoA; malonyl-CoA; OAC; and one or more of: olivetolic acid, olivetol, and/or pentyl diacetic acid lactone, as compared to an OLS from C. sativa under the same reaction conditions.

37. An engineered cell comprising an olivetol synthase (OLS) of any of SEQ ID NOs:2-49, wherein the cell is a bacterial cell.

38. The engineered cell of claim 37, wherein the OLS comprises any of SEQ ID NOs:2, 3, 4, 6, 7, 8, 9, 11, 13, 14, 15, or 20.

39. The engineered cell of claim 37, wherein the OLS comprises any of SEQ ID NOs:4, 6, 8, 9, 11, 13, 15, or 20.

40. The engineered cell of claim 37, wherein the OLS comprises any of SEQ ID NOs:2, 3, 6, or 8.

41. The engineered cell of claim 37, wherein the OLS comprises any of SEQ ID NOs:6 or 8.

42. The engineered cell of claim 37, wherein the OLS comprises SEQ ID NO:2.

43. The engineered cell of claim 37, wherein the OLS comprises SEQ ID NO:6.

44. The engineered cell of any one of claims 37 to 43, wherein the OLS comprises an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

45. An engineered cell comprising a non-naturally occurring olivetol synthase (OLS), wherein the OLS comprises at least 90% sequence identity to any of SEQ ID NOs: 10-45, SEQ ID NO:48, or SEQ ID NO:49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

46. An engineered cell comprising a non-naturally occurring olivetol synthase (OLS) comprising at least 95% sequence identity to SEQ ID NO:2 or any of SEQ ID NO:4-49, and further comprising an amino acid variation at an amino acid position corresponding to amino acid position 70, 133, 134, 160, 161, 192, 193, 194, 195, 196, 198, 207, 208, 214, 216, 218, 255, 259, 264, 266, 267, 268, 269, 303, 305, 338, 339, 340, 373, 374, and/or 380 of SEQ ID NO:6.

47. The engineered cell of any one of claims 44 to 46, wherein the amino acid variation is an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

48. The engineered cell of claim 47, wherein the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof.

49. The engineered cell of any one of claims 44 to 46, wherein the amino acid variation is an amino acid substitution, wherein the amino acid substitution comprises F70M, Y160G, Q161F, T195V, E207S, D208A, D208S, D208N, D208C, I255M, L264F, H269S, P303A, P303V, P305N, S339W, G373A, F374L, or a combination thereof, wherein the amino acid position corresponds to SEQ ID NO:6.

50. An engineered cell comprising a non-naturally occurring olivetol synthase (OLS) comprising at least 90% sequence identity to any of SEQ ID NOs:2-49, and further comprising an amino acid variation at an amino acid position corresponding to amino acid position 133, 134, 192, 193, 194, 196, 198, 214, 216, 218, 259, 266, 267, 268, 338, 340, and/or 380 of SEQ ID NO:6.

51. The engineered cell of claim 50, wherein the amino acid variation is an amino acid substitution, wherein the amino acid substitution comprises S133A, S133G, S133W, G134H, E192D, T193S, T194A, T194E, T194N, T194Q, T194S, V196C, F198L, L214M, A216G, G218A, V259Q, V259W, V259Y, A266P, T267I, T267V, T267W, T267Y, L268M, L268V, M338L, M338T, S340A, V380L, or a combination thereof, wherein the amino acid position corresponds to SEQ ID NO:6.

52. An engineered cell comprising a non-naturally occurring olivetol synthase (OLS) comprising at least 90% sequence identity to any of SEQ ID NOs:2-49, and further comprising an amino acid substitution, wherein the amino acid substitution comprises F70N, F70Q, F70V, S133A, S133G, S133W, G134H, Q161H, Q161M, Q161T, Q161L, Q161Y, Q161W, Q161V, Q161G, Q161F, E192D, T193S, T194A, T194E, T194N, T194Q, T194S, T195M, V196C, F198L, E207C, D208H, L214M, A216G, G218A, I255L, I255S, I255M, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof, wherein the amino acid position corresponds to SEQ ID NO:6.

53. The engineered cell of claim 52, wherein the amino acid substitution comprises F70N, F70Q, S133A, S133G, S133W, G134H, Q161H, Q161M, Q161T, E192D, T193S, T194A, T194E, T194N, T194Q, T194S, T195M, V196C, F198L, E207C, D208H, L214M, A216G, G218A, I255L, V259Q, V259W, V259Y, A266P, T267I, T267V, T267W, T267Y, L268M, L268V, H269T, P303A, P303C, P303I, P303L, P303M, P303T, P303V, P305L, M338L, M338T, S340A, F374I, F374M, F374V, V380L, or a combination thereof.

54. The engineered cell of any one of claims 50 to 53, comprising at least 90% sequence identity to SEQ ID NO:2 or 6.

55. The engineered cell of any one of claims 46 to 54, comprising at least 97% sequence identity to SEQ ID NO:2 or 6.

56. An engineered cell comprising: the polynucleotide of any one of claims 1 to 13, claim 28, or claim 29; the OLS of any one of claims 14 to 27 or any one of claims 32 to 36; and/or the expression construct of claim 30 or 31.

57. The engineered cell of claim 56, wherein the cell comprises the polynucleotide, and wherein the polynucleotide is integrated into a genome of the cell.

58. The engineered cell of claim 56, wherein the cell comprises the polynucleotide, and wherein the polynucleotide is present on an expression construct.

59. The engineered cell of any one of claims 37 to 55, further comprising a cannabinoid biosynthesis pathway enzyme and/or a polynucleotide encoding a cannabinoid biosynthesis pathway enzyme.

60. The engineered cell of claim 59, wherein the cannabinoid biosynthesis pathway enzyme comprises olivetolic acid cyclase (OAC), prenyltransferase, a cannabinoid synthase, a geranyl pyrophosphate (GPP) biosynthesis pathway enzyme, or combination thereof.

61. The engineered cell of claim 60, wherein the OAC comprises an amino acid substitution at amino acid position H5,17, L9, F23, F24, Y27, V46, T47, Q48, K49, N50, K51, V59, V61, V66, E67, 169, Q70, 173, 174, V79, G80, F81, G82, D83, R86, W89, L92, 194, D96, or a combination thereof, wherein the amino acid position is relative to SEQ ID NO:50.

62. The engineered cell of claim 60 or 61, wherein the prenyltransferase comprises an amino acid substitution at amino acid position V45, V47, S49, F121, T124, Q159, M160, Y173, S212, V213, A230, 1232, T267, V269, Y286, T290, Q293, R294, L296, F300, or a combination thereof, wherein the amino acid position is relative to SEQ ID NO:51.

63. The engineered cell of any one of claims 60 to 62, wherein the cannabinoid synthase comprises tetrahydrocannabinolic acid synthase (THCAS), cannabidiolic acid synthase (CBDAS), cannabichromenic acid synthase (CBCAS), or combination thereof.

64. The engineered cell of any one of claims 60 to 63, wherein the GPP biosynthesis pathway enzyme comprises geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, or a combination thereof.

65. The engineered cell of any one of claims 45 to 64, wherein the cell is a bacterial cell.

66. The engineered cell of any one of claims 37 to 64, wherein the cell is an Escherichia coli cell.

67. The engineered cell of any one of claims 37 to 66, wherein the cell is capable of producing 3,5,7-trioxododecanoyl-CoA, olivetolic acid, a cannabinoid, an analog or derivative thereof; or a combination thereof.

68. The engineered cell of claim 67, wherein the cell is further capable of producing olivetol;
pentyl diacetic acid lactone (PDAL); hexanoyl triacetic acid lactone (HTAL); an analog or derivative thereof; or a combination thereof.

69. A cell extract or cell culture medium comprising 3,5,7-trioxododecanoyl-CoA, olivetol, olivetolic acid, a cannabinoid, and/or an isomer, analog, or derivative thereof, wherein the cell culture extract or medium is derived from the engineered cell of any one of claims 37 to 68.

70. A method of making 3,5,7-trioxododecanoyl-CoA, olivetol, olivetolic acid, a cannabinoid, and/or an isomer, analog, or derivative thereof, comprising:
culturing the engineered cell of any one of claims 37 to 68; and/or
isolating the 3,5,7-trioxododecanoyl-CoA, olivetol, olivetolic acid, cannabinoid, or isomer, analog, or derivative thereof from the cell extract of cell culture medium of claim 69.

71. The method of claim 70, wherein the engineered cell is cultured in the presence of hexanoic acid.

72. A composition comprising 3,5,7-trioxododecanoyl-CoA, olivetol, olivetolic acid, a cannabinoid, and/or an isomer, analog, or derivative thereof, wherein the 3,5,7-trioxododecanoyl-CoA, olivetol, olivetolic acid, cannabinoid, and/or isomer, analog, or derivative thereof is produced by the engineered cell of any one of claims 37 to 68; isolated from the cell extract or cell culture medium of claim 69; and/or made by the method of claim 70 or 71.

73. The composition of claim 72, wherein the composition comprises a cannabinoid selected from cannabigerolic acid (CBGA), tetrahydrocannabinolic acid (THCA), cannabidiolic acid (CBDA), cannabichromenic acid (CBCA), cannabigerol (CBG), tetrahydrocannabinol (THC), cannabidiol (CBD), cannabichromene (CBC), an isomer, analog, or derivative thereof, or a combination thereof.

74. The composition of claim 73, wherein the composition is a therapeutic or medicinal composition, an oral unit dosage composition, a topical composition, or an edible composition.

75. A cannabinoid produced by the engineered cell of any one of claims 37 to 68; isolated from the cell extract or cell culture medium of claim 69; and/or made by the method of claim 70 or 71.

76. The cannabinoid of claim 75, wherein the cannabinoid is cannabigerolic acid (CBGA), tetrahydrocannabinolic acid (THCA), cannabidiolic acid (CBDA), cannabichromenic acid (CBCA), cannabigerol (CBG), tetrahydrocannabinol (THC), cannabidiol (CBD), cannabichromene (CBC), an isomer, analog, or derivative thereof, or a combination thereof.

77. A composition comprising: (a) the OLS of any one of claims 14 to 27; and (b) hexanoyl-CoA, malonyl-CoA, 3,5,7-trioxododecanoyl-CoA, olivetol, PDAL, an analog, isomer, or derivative thereof, or a combination thereof.
